(19) United States
(12) Patent Application Publication
(10) Pub. No.: US 2025/0114438 A1
(43) Pub. Date: Apr. 10, 2025

(54) METHOD FOR TREATING TUMOR USING IMMUNE CELL

(71) Applicant: CARSGEN THERAPEUTICS CO., LTD., Shanghai (CN)

(72) Inventors: Zonghai LI, Shanghai (CN); Huiping GAO, Shanghai (CN); Hua JIANG, Shanghai (CN); Huamao WANG, Shanghai (CN)

(73) Assignee: CARSGEN THERAPEUTICS CO., LTD., Shanghai (CN)

(21) Appl. No.: 18/908,061

(22) Filed: Oct. 7, 2024

(30) Foreign Application Priority Data
Oct. 7, 2023 (CN) 202311287919.9

(51) Int. Cl. A61K 39/00; A61K 9/00; A61K 31/52; A61K 31/675; A61K 38/38

(52) U.S. Cl. CPC A61K 39/00; A61K 9/0019; A61K 31/52; A61K 31/675; A61K 38/38; A61K 2039/5156

(57) ABSTRACT

The present application provides a new adjuvant treatment method for treating tumors, reducing tumor recurrence, inhibiting tumor growth, or inducing tumor necrosis, wherein the method comprises the following: after selecting patients with cancer (for example, liver cancer, pancreatic cancer, gastric cancer, or gastroesophageal junction tumor) in need thereof for tumor local treatment, a therapeutically effective amount of immune cells (for example, CAR-T cells) is administered as a new adjuvant treatment.

### FIELD OF THE INVENTION

The present application relates to a method for treating solid tumors with immune cells expressing chimeric receptors.

### BACKGROUND OF THE INVENTION

Liver cancer is one of the most common malignant tumors of digestive system. About half of the patients with liver cancer were complicated with large vessel invasion and/or vascular tumor thrombus. Portal vein tumor thrombus (PVTT) is the most common form of large vessel invasion, and about 44%-62.2% of liver cancer patients are complicated with PVTT. Vascular invasion is an important risk factor for postoperative recurrence of liver cancer. Even after successful resection of liver cancer, most patients will have tumor recurrence within one year. The median recurrence free survival and median overall survival are only 4-9 months and 14-21.9 months, respectively. Postoperative adjuvant treatments may be a potentially effective means to prevent tumor recurrence.

Gastric cancer is one of the cancers with high incidence worldwide. Most patients were in advanced stage or late stage at diagnosis, and the overall prognosis was poor. Radical surgery combined with postoperative adjuvant chemotherapy is the current standard treatment for locally advanced gastric cancer. Surgical resection is the main means to cure gastric cancer. Postoperative tumor recurrence is a key factor affecting the prognosis of patients.

Pancreatic cancer with extremely high mortality has the characteristics of strong metastatic ability, high recurrence rate and poor prognosis. Surgery is the main means to cure pancreatic cancer, but because most patients appear late in clinical manifestations, only 15%-20% of patients are suitable for pancreatic resection. However, most patients also relapse within a year, and 60% of pancreatic cancer patients develop liver metastasis within 2 years after surgery.

Therefore, it is urgent to further explore how to optimize adjuvant treatment strategies to improve patient survival outcomes in tumor patients with high recurrence risk.

### SUMMARY OF THE INVENTION

The present application provides an adjuvant treatment method for treating solid tumors. The first aspect provides an adjuvant treatment method, comprising administering the adjuvant treatment to a subject with the solid tumor after receiving local treatment; and the adjuvant treatment comprises: administering a therapeutically effective amount of immune cell therapy comprising a chimeric receptor to the subject by intravenous infusion.

In one example, the local treatment reduces or eliminates tumor burden.

In one example, the tumor burden comprises: the number of tumor lesions, the size of tumor lesions, and/or changes in the level of tumor markers.

In one example, the local treatment comprises surgical treatment and/or interventional treatment.

In one example, the local treatment comprises: surgical treatment, cryoablation, microwave treatment, vascular embolization, radiofrequency treatment, gamma knife treatment, focused ultrasound treatment, photodynamic treatment, argon-helium knife treatment, radioactive particle implantation, or any combination thereof.

In one example, the tumor surgical treatment comprises radical tumor surgery or palliative tumor surgery.

In one example, after the local treatment combined with other adjuvant treatment, the immune cell therapy is administered; and the other adjuvant treatment does not comprise the immune cell therapy.

In one example, the other adjuvant treatment is administered before and/or after local treatment.

In one example, after the local treatment or the local treatment combined with the other adjuvant treatment, the immune cell therapy is administered.

In one example, after the local treatment or the local treatment combined with the other adjuvant treatment, the immune cell therapy is administered if no enlargement of other small tumor lesions without the local treatment is detected by imaging technology.

In one example, after the local treatment or the local treatment combined with the other adjuvant treatment, the immune cell therapy is administered if the tumor marker level is detected to be higher than the upper limit of the normal range or higher than the baseline level, or the tumor marker level is detected to be progressively increased.

In one example, after the local treatment or the local treatment combined with the other adjuvant treatment, the immune cell therapy is administered if circulating tumor DNA (ctDNA) or a circulating tumor cell (CTC) are detected in peripheral blood.

In one example, after the local treatment or the local treatment combined with the other adjuvant treatment, the immune cell therapy is administered if the subject is in progression free survival (PFS) of the disease according to the efficacy evaluation.

In one example, the method is used to treat a subject with liver cancer. After the local treatment or the local treatment combined with the other adjuvant treatment, CAR-T cells binding to GPC3, NKG2DL, EpCAM, and/or B7H3, or TCR-T cells binding to HBsAg or AFP is administered by intravenous infusion if the AFP level in peripheral blood is detected to be higher than the upper limit of the normal range or higher than the baseline level, or the AFP level is progressively increased.

In one example, the method is used to treat a subject with liver cancer. After the local treatment or the local treatment combined with the other adjuvant treatment, CAR-T cells binding to GPC3, NKG2DL, EpCAM, and/or B7H3, or TCR-T cells binding to HBsAg or AFP is administered by intravenous infusion if no tumor recurrence and/or metastasis is detected by imaging technology.

In one example, the method is used to treat a subject with GPC3-positive liver cancer. After the local treatment or the local treatment combined with the other adjuvant treatment, GPC3-CAR-T cells are administered by intravenous infusion if no tumor recurrence and/or metastasis is detected by imaging technology.

In one example, the method is used to treat a subject with GPC3-positive liver cancer. After the local treatment or the local treatment combined with the other adjuvant treatment, GPC3-CAR-T cells are administered by intravenous infusion if AFP in peripheral blood is detected to be higher than the upper limit of the normal range or higher than the baseline level, or the AFP level is progressively increased.

In one example, the method is used to treat subjects with GPC3-positive liver cancer. After the local treatment or the local treatment combined with the other adjuvant treatment, GPC3-CAR-T cells are administered by intravenous infusion if the tumor marker level is detected to be higher than the upper limit of the normal range or higher than the baseline level, or the tumor marker level is progressively increased, and no tumor recurrence and/or metastasis is detected by imaging technology.

In one example, the method is used to treat subjects with CLDN18.2-positive solid tumors. After the local treatment or the local treatment combined with the other adjuvant treatment, CLDN18.2-CAR-T cells are administered by intravenous infusion if no tumor recurrence and/or metastasis is detected by imaging technology.

In one example, the method is used to treat a subject with gastric cancer or gastroesophageal tumor. After the local treatment or the local treatment combined with the other adjuvant treatment, CAR-T cells binding to CLDN18.2, EpCAM, B7H3, and/or NKG2DL are administered by intravenous infusion if no tumor recurrence and/or metastasis is detected by imaging technology.

In one example, the method is used to treat subjects with CLDN18.2-positive gastric cancer or gastroesophageal junction tumor. After the local treatment or the local treatment combined with the other adjuvant treatment, CLDN18.2-CAR-T cells are administered by intravenous infusion if no tumor recurrence and/or metastasis is detected by imaging technology.

In one example, the method is used to treat a subject with pancreatic cancer. After the local treatment or the local treatment combined with the other adjuvant treatment, CAR-T cells binding to MSLN, MUC1, HER2, CEA, PSCA, CLDN18.2, EpCAM, B7H3, and/or NKG2DL are administered by intravenous infusion if the tumor marker level is detected to be higher than the upper limit of the normal range or higher than the baseline level, or the tumor marker level is progressively increased.

In one example, the method is used to treat a subject with pancreatic cancer. After the local treatment or the local treatment combined with the other adjuvant treatment, CAR-T cells binding to MSLN, MUC1, HER2, CEA, PSCA, CLDN18.2, EpCAM, B7H3, and/or NKG2DL are administered by intravenous infusion if no tumor recurrence and/or metastasis is detected by imaging technology.

In one example, the method is used to treat a subject with pancreatic cancer. After the local treatment or the local treatment combined with the other adjuvant treatment, CAR-T cells binding to MSLN, MUC1, HER2, CEA, PSCA, CLDN18.2, EpCAM, B7H3, and/or NKG2DL are administered by intravenous infusion if the CA19-9 level in peripheral blood is detected to be higher than the upper limit of the normal range or higher than the baseline level, or the CA19-9 level is progressively increased, and no tumor recurrence and/or metastasis is detected by imaging technology.

In one example, the method is used to treat subjects with CLDN18.2-positive pancreatic cancer. After the local treatment or the local treatment combined with the other adjuvant treatment, CLDN18.2-CAR-T cells are administered by intravenous infusion if no tumor recurrence and/or metastasis is detected by imaging technology.

In one example, the method is used to treat subjects with CLDN18.2-positive pancreatic cancer. After the local treatment or the local treatment combined with the other adjuvant treatment, CLDN18.2-CAR-T cells are administered by intravenous infusion if the tumor marker level is detected to be higher than the upper limit of the normal range or higher than the baseline level, or the tumor marker level is progressively increased.

In one example, the method is used to treat a subject with CLDN18.2-positive pancreatic cancer. After the local treatment or the local treatment combined with the other adjuvant treatment, CLDN18.2-CAR-T cells are administered by intravenous infusion if the CA19-9 level in peripheral blood is detected to be higher than the upper limit of the normal range or higher than the baseline level, or the CA19-9 level is progressively increased, and no tumor recurrence and/or metastasis is detected by imaging technology.

In one example, the other adjuvant treatment comprises systemic chemotherapy.

In one example, the other adjuvant treatment comprises gemcitabine+capecitabine, or gemcitabine+albumin-bound paclitaxel or mFOLFIRINOX.

The second aspect provides an adjuvant treatment method, comprising administering the adjuvant treatment to a subject with a solid tumor after the radical tumor surgery; and the adjuvant treatment comprises: administering a therapeutically effective amount of immune cell therapy comprising a chimeric receptor to the subject by intravenous infusion.

In one example, after the radical surgery combined with the other adjuvant treatment, the immune cell therapy is administered; and the other adjuvant treatment does not comprise the immune cell therapy.

In one example, the other adjuvant treatment is administered before and/or after radical surgery.

In one example, after the radical surgery or the radical surgery combined with the other adjuvant treatment, the immune cell therapy is administered.

In one example, after the radical surgery or the radical surgery combined with the other adjuvant treatment, the immune cell therapy is administered if no enlargement of other small tumor lesions without local treatment is detected by imaging technology.

In one example, after the radical surgery or the radical surgery combined with the other adjuvant treatment, the immune cell therapy is administered if the tumor marker level is detected to be higher than the upper limit of the normal range or higher than the baseline level, or the tumor marker level is progressively increased.

In one example, after the radical surgery or the radical surgery combined with the other adjuvant treatment, the immune cell therapy is administered if circulating tumor DNA (ctDNA) or circulating tumor cell (CTC) are detected in peripheral blood.

In one example, after the radical surgery or the radical surgery combined with the other adjuvant treatment, the immune cell therapy is administered if the subject is in a non progressive stage of disease according to the efficacy evaluation.

In one example, the method is used to treat a subject with liver cancer. After the radical surgery or the radical surgery combined with the other adjuvant treatment, CAR-T cells binding to GPC3, NKG2DL, EpCAM, and/or B7H3, or TCR-T cells binding to HBsAg or AFP are administered by intravenous infusion if no tumor recurrence and/or metastasis is detected by imaging technology.

In one example, the method is used to treat subjects with GPC3-positive solid tumors. After the radical surgery or the radical surgery combined with the other adjuvant treatment, GPC3-CAR-T cells are administered by intravenous infusion if no tumor recurrence and/or metastasis is detected by imaging technology.

In one example, the method is used to treat subjects with GPC3-positive solid tumors. After the radical surgery or the radical surgery combined with the other adjuvant treatment, GPC3-CAR-T cells are administered by intravenous infusion if the tumor marker level is detected to be higher than the upper limit of the normal range or higher than the baseline level, or the tumor marker level is progressively increased.

In one example, the method is used to treat subjects with GPC3-positive solid tumors. After the radical surgery or the radical surgery combined with the other adjuvant treatment, GPC3-CAR-T cells are administered by intravenous infusion if the tumor marker level is detected to be higher than the upper limit of the normal range or higher than the baseline level, or the tumor marker level is progressively increased, and no tumor recurrence and/or metastasis is detected by imaging technology.

In one example, the method is used to treat a subject with liver cancer. After the radical surgery or the radical surgery combined with the other adjuvant treatment, CAR-T cells binding to GPC3, NKG2DL, EpCAM, and/or B7H3, or TCR-T cells binding to HBsAg or AFP are administered by intravenous infusion if the AFP level in peripheral blood is detected to be higher than the upper limit of the normal range or higher than the baseline level, or the AFP level is progressively increased. In one example, the method is used to treat a subject with liver cancer. After the radical surgery or the radical surgery combined with the other adjuvant treatment, CAR-T cells binding to GPC3, NKG2DL, EpCAM, and/or B7H3, or TCR-T cells binding to HBsAg or AFP are administered by intravenous infusion if no tumor recurrence and/or metastasis is detected by imaging technology.

In one example, the method is used to treat subjects with GPC3-positive liver cancer. After the radical surgery or the radical surgery combined with the other adjuvant treatment, GPC3-CAR-T cells are administered by intravenous infusion if no tumor recurrence and/or metastasis is detected by imaging technology.

In one example, the method is used to treat subjects with GPC3-positive liver cancer. After the radical surgery or the radical surgery combined with the other adjuvant treatment, GPC3-CAR-T cells are administered by intravenous infusion if the AFP level in peripheral blood is detected to be higher than the upper limit of the normal range or higher than the baseline level, or the AFP level is progressively increased.

In one example, the method is used to treat subjects with GPC3-positive liver cancer. After the is radical surgery or the radical surgery combined with the other adjuvant treatment, GPC3-CAR-T cells are administered by intravenous infusion if the tumor marker level is detected to be higher than the upper limit of the normal range or higher than the baseline level, or the tumor marker level is progressively increased, and no tumor recurrence and/or metastasis is detected by imaging technology.

In one example, the method is used to treat subjects with CLDN18.2-positive solid tumors. After the radical surgery or the radical surgery combined with the other adjuvant treatment, CLDN18.2-CAR-T cells are administered by intravenous infusion if no tumor recurrence and/or metastasis is detected by imaging technology.

In one example, the method is used to treat a subject with gastric cancer or gastroesophageal tumor. After the radical surgery or the radical surgery combined with the other adjuvant treatment, CAR-T cells binding to CLDN18.2, EpCAM, B7H3, and/or NKG2DL are administered by intravenous infusion if no tumor recurrence and/or metastasis is detected by imaging technology.

In one example, the method is used to treat subjects with CLDN18.2-positive gastric cancer or gastroesophageal junction tumor. After the radical surgery or the radical surgery combined with the other adjuvant treatment, CLDN18.2-CAR-T cells are administered by intravenous infusion if no tumor recurrence and/or metastasis is detected by imaging technology.

In one example, the method is used to treat a subject with pancreatic cancer. After the radical surgery or the radical surgery combined with the other adjuvant treatment, CAR-T cells binding to MSLN, MUC1, HER2, CEA, PSCA, CLDN18.2, EpCAM, B7H3, and/or NKG2DL are administered by intravenous infusion if the tumor marker level is detected to be higher than the upper limit of the normal range or higher than the baseline level, or the tumor marker level is progressively increased.

In one example, the method is used to treat a subject with pancreatic cancer. After the radical surgery or the radical surgery combined with the other adjuvant treatment, CAR-T cells binding to MSLN, MUC1, HER2, CEA, PSCA, CLDN18.2, EpCAM, B7H3, and/or NKG2DL are administered by intravenous infusion if no tumor recurrence and/or metastasis is detected by imaging technology.

In one example, the method is used to treat a subject with pancreatic cancer. After the radical surgery or the radical surgery combined with the other adjuvant treatment, CAR-T cells binding to MSLN, MUC1, HER2, CEA, PSCA, CLDN18.2, EpCAM, B7H3, and/or NKG2DL are administered by intravenous infusion if the CA19-9 level in peripheral blood is detected to be higher than the upper limit of the normal range or higher than the baseline level, or the CA19-9 level is progressively increased, and no tumor recurrence and/or metastasis is detected by imaging technology.

In one example, the method is used to treat subjects with CLDN18.2-positive pancreatic cancer. After the radical surgery or the radical surgery combined with the other adjuvant treatment, CLDN18.2-CAR-T cells are administered by intravenous infusion if no tumor recurrence and/or metastasis is detected by imaging technology.

In one example, the method is used to treat subjects with CLDN18.2-positive pancreatic cancer. After the radical surgery or the radical surgery combined with the other adjuvant treatment, CLDN18.2-CAR-T cells are administered by intravenous infusion if the tumor marker level is detected to be higher than the upper limit of the normal range or higher than the baseline level, or the tumor marker level is progressively increased.

In one example, the method is used to treat subjects with CLDN18.2-positive pancreatic cancer. After the radical surgery or the radical surgery combined with the other adjuvant treatment, CLDN18.2-CAR-T cells are administered by intravenous infusion if the CA19-9 level in peripheral blood is detected to be higher than the upper limit of the normal range or higher than the baseline level, or the CA19-9 level is progressively increased, and no tumor recurrence and/or metastasis is detected by imaging technology.

In one example, in the adjuvant treatment method provided by the first or second aspect above, the chimeric receptor binds to the tumor antigen expressed by the solid tumor.

In one example, in the adjuvant treatment method provided by the first or second aspect above, the chimeric receptor binds the tumor antigen expressed on the cell membrane of the solid tumor.

In one example, in the adjuvant treatment method provided in the first or second aspect, the other adjuvant treatment comprises: chemotherapy, radiotherapy, hormone therapy, immune checkpoint inhibitor therapy, immune modulator therapy, antibody therapy, antiangiogenic drug therapy, small molecule compound therapy, or any combinations thereof.

In one example, in the adjuvant treatment method provided by the first or second aspect above, antibody treatment comprises: administration of monoclonal antibody, antibody-drug conjugate treatment, bifunctional antibody, and/or multifunctional antibody.

In one example, in the adjuvant treatment method provided by the first or second aspect above, the other adjuvant treatment comprises systemic chemotherapy.

In one example, in the adjuvant treatment method provided by the first or second aspect above, the other adjuvant treatment comprises CapeOX, FOLFOX, FLOT, gemcitabine+capecitabine, gemcitabine+albumin-bound paclitaxel or mFOLFIRINOX.

In one example, in the adjuvant treatment method provided by the first or second aspect above, the other adjuvant treatment comprises anti-PD-1, PDL-1 and/or VEGFR antibody treatment.

In one example, in the adjuvant treatment method provided by the first or second aspect above, the other adjuvant treatment comprises: (a) one or more inhibitors of checkpoint molecules, which comprise PD-1, PDL-1, TIM-3, TIGIT, LAG-3, CTLA-4, 2B4, 4-1BB, 4-1BBL, A2aR, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E or inhibitory KIR;
(b) one or more of atezolizumab, avelumab, durvalumab, ipilimumab, IPH4102, IPH43, IPH33, lirimumab, monalizumab, nivolumab, pembrolizumab, and derivatives or functional equivalents thereof; or (c) at least one of atezolizumab, nivolumab, and pembrolizumab; or (d) the therapeutic agent comprises one or more of venetoclax, azacitidine, and pomalidomide.

In one example, in the adjuvant treatment method provided by the first or second aspect above, no tumor recurrence and/or metastasis are detected by imaging, histology and/or cytopathological techniques.

In one example, in the adjuvant treatment method provided by the first or second aspect above, the PFS comprises SD, PR or CR.

In one example, in the adjuvant treatment method provided by the first or second aspect above, the efficacy evaluation is evaluated with reference to RECIST.

In one example, in the adjuvant treatment method provided by the first or second aspect above, the efficacy evaluation is evaluated with reference to RECIST v1.1.

In one example, in the adjuvant treatment method provided by the first or second aspect above, the chimeric receptor comprises a chimeric antigen receptor (CAR) and/or a recombinant TCR.

In one example, in the adjuvant treatment method provided by the first or second aspect above, the chimeric receptor comprises an extracellular domain, a transmembrane domain, and an intracellular domain.

In one example, in the adjuvant treatment method provided by the first or second aspect above, the chimeric receptor comprises at least one, two or three intracellular signaling domains.

In one example, in the adjuvant treatment method provided by the first or second aspect above, the intracellular signaling domain comprises the signaling domain of CD3, CD28, 4-1BB, OX40, DAP10 or ICOS.

In one example, in the adjuvant treatment method provided by the first or second aspect above, the transmembrane domain comprises the transmembrane domain of a natural marker on the surface of T cells.

In one example, in the adjuvant treatment method provided by the first or second aspect above, the transmembrane domain comprises a CD8 transmembrane domain or a CD28 transmembrane domain.

In one example, in the adjuvant treatment method provided by the first or second aspect above, the transmembrane domain comprises a CD8 α transmembrane domain.

In one example, in the adjuvant treatment method provided by the first or second aspect above, the transmembrane domain and intracellular signaling domain of the chimeric receptor are from the same molecule.

In one example, in the adjuvant treatment method provided by the first or second aspect above, the transmembrane domain and intracellular signaling domain of the chimeric receptor are from different molecules.

In one example, in the adjuvant treatment method provided by the first or second aspect above, the extracellular domain of the chimeric receptor is bound to the transmembrane domain through the hinge region.

In one example, in the adjuvant treatment method provided by the first or second aspect above, the hinge region and the transmembrane domain are from the same molecule.

In one example, in the adjuvant treatment method provided by the first or second aspect above, the hinge region and the transmembrane domain are from different molecules.

In one example, in the adjuvant treatment method provided by the first or second aspect above, the hinge region comprises a CD8 hinge region, an IgGI hinge region, an IgG4 hinge region, or a CD28 hinge region.

In one example, in the adjuvant treatment method provided by the first or second aspect above, the hinge region comprises a CD8 α hinge region.

In one example, in the adjuvant treatment method provided by the first or second aspect above, the CAR comprises:
(1) an antigen binding unit that binds to tumor antigens, CD8 or CD28 transmembrane domain, CD3ζ intracellular domain; (2) an antigen binding unit that binds to tumor antigen, CD8 or CD28 transmembrane domain, 4-1BB intracellular domain, CD3ζ intracellular domain; (3) an antigen binding unit that binds to tumor antigen, CD8 or CD28 transmembrane domain, CD28 intracellular domain, CD3ζ intracellular domain; or (4) an antigen binding unit that binds to tumor antigen, CD8 or CD28 transmembrane domain, CD28 intracellular domain, 4-1BB intracellular domain, CD3ζ intracellular domain.

In one example, in the adjuvant treatment method provided by the first or second aspect above, the CAR comprises:
(1) an antibody variable region that binds to tumor antigens, CD8 or CD28 transmembrane domain, CD3ζ intracellular domain;
(2) an antibody variable region that binds to tumor antigens, CD8 or CD28 transmembrane domain, CD137 intracellular domain, CD3ζ intracellular domain;
(3) an antibody variable region that binds to tumor antigens, CD8 or CD28 transmembrane domain, CD28 intracellular domain, CD3ζ intracellular domain; or
(4) an antibody variable region that binds to tumor antigens, CD8 or CD28 transmembrane domain, CD28 intracellular domain, CD137 intracellular domain, CD3ζ intracellular domain.

In one example, in the adjuvant treatment method provided by the first or second aspect above, the CAR comprises:
(1) scFv antibody that binds to tumor antigens, CD8 or CD28 transmembrane domain, CD3ζ intracellular domain;
(2) scFv antibody that binds to tumor antigens, CD8 or CD28 transmembrane domain, CD137 intracellular domain, CD3ζ intracellular domain;
(3) scFv antibody that binds to tumor antigens, CD8 or CD28 transmembrane domain, CD28 intracellular domain, CD3ζ intracellular domain; or
(4) scFv antibody that binds to tumor antigens, CD8 or CD28 transmembrane domain, CD28 intracellular domain, CD137 intracellular domain, CD3ζ intracellular domain;

In one example, in the adjuvant treatment method provided by the first or second aspect above, the immune cells comprise: T cells, NK cells, natural killer T cells (NKT), human embryonic stem cells, or pluripotent stem cells.

In one example, in the adjuvant treatment method provided by the first or second aspect above, the immune cells are autologous or allogeneic cells.

In one example, in the adjuvant treatment method provided by the first or second aspect above, the endogenous TCR and B2M of the immune cells are low or not expressed.

In one example, in the adjuvant treatment method provided by the first or second aspect above, gene editing technology, including CRISPR/Cas technology, ZFN technology, TALEN technology and TALEN-CRISPR/Cas technology, base editor technology, prime editor technology and meganuclease technology, is used to knock out endogenous TCR and B2M genes.

In one example, in the adjuvant treatment method provided by the first or second aspect above, the recombinant nucleic acid sequence of the chimeric receptor is integrated in the first exon of the constant region of the TCRα chain.

In one example, in the adjuvant treatment method provided by the first or second aspect above, the recombinant nucleic acid sequence of the chimeric receptor is integrated in the first exon of the constant region of the TCRα chain, and the integration reduces endogenous TCR expression, and the expression of the integrated nucleic acid sequence in T cells is under the control of the endogenous TCRα chain promoter or the exogenous promoter.

In one example, in the adjuvant treatment method provided by the first or second aspect above, CRISPR technology is used to knock out endogenous TCR/B2M, TCR/B2M/FAS, TCR/B2M/CD38, TCR/B2M/CD38/NKG2A, TCR/B2M/NKG2A, TCR/B2M/FAS/CD38, or TCR/B2M/FAS/NKG2A.

In one example, in the adjuvant treatment method provided by the first or second aspect above, the immune cell further comprises a second polypeptide: an immune response inhibitor, a cytokine, a cytokine receptor, a chemokine, or a chemokine receptor, or a combination thereof.

In one example, in the adjuvant treatment method provided by the first or second aspect above, the cytokines comprise: lymphokines, monokine, interleukins (IL), such as IL-1, IL-la, IL-2, IL-3, IL-4, IL-5, IL-6, IL-8, IL-9, IL-11, IL-12, IL15, IL-21, IL-18, IL-9, IL-23, IL-36γ; interferon IFNα2b; tumor necrosis factors, such as TNF-α or TNF-β; variants and combinations thereof.

In one example, in the adjuvant treatment method provided by the first or second aspect above, the cytokines comprise: IL15, IL21, IL12, IL7, or a combination thereof.

In one example, in the adjuvant treatment method provided by the first or second aspect above, the second polypeptide comprises: the ligand binding domain, and transmembrane domain of the receptor for interleukin-13 receptor α2 (IL13Rα2), interleukin-2 receptor β (IL2Rβ), interleukin-18 receptor α (IL18Ru) or interleukin-18 receptor β (IL18Rβ), and the intracellular signaling domain of interleukin-9 receptor α (IL9Rα).

In one example, in the adjuvant treatment method provided by the first or second aspect above, the cytokine receptors comprise: IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, variants and combinations thereof.

In one example, in the adjuvant treatment method provided by the first or second aspect above, the chemokine receptor comprises: a naturally occurring or recombinant chemokine receptor or a chemokine binding fragment thereof.

In one example, in the adjuvant treatment method provided by the first or second aspect above, the chemokine receptor comprises: CXC chemokine receptor (e.g., CXCR1, CXCR2, CXCR3, CXCR4, CXCR5, CXCR6, or CXCR7), CC chemokine receptor (e.g., CCR1, CCR2, CCR3, CCR4, CCR5, CCR6, CCR7, CCR8, CCR9, CCR10, or CCR11), CX3C chemokine receptor (e.g., CX3CR1), XC chemokine receptor (e.g., XCR1), or chemokine binding fragment thereof.

In one example, in the adjuvant treatment method provided by the first or second aspect above, the chemokine receptor comprises: CCR4, CXCR5, CXCL13, CCR2b, or CXCR2, or a combination thereof.

In one example, in the adjuvant treatment method provided by the first or second aspect above, the chemokine is selected from CCL2, CCL5, CCL10, CCL14, CCL19, CCL20, CCL21, CXCL8, CXCL9, CXCL13, or LEC, or a combination thereof; preferably, the chemokine is selected from CXCL9, CCL19, or CCL21, or a combination thereof.

In one example, in the adjuvant treatment method provided by the first or second aspect above, the immune response inhibitor is an immune checkpoint inhibitor, the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the inhibitor is preferably PD-1. In certain embodiments, the inhibitor is transforming growth factor β(TGF-β) receptor.

In one example, in the adjuvant treatment method provided by the first or second aspect above, the immune response inhibitor is a dominant negative TGF-β receptor.

In one example, in the adjuvant treatment method provided by the first or second aspect above, the intracellular signaling domain of the dominant negative TGF-β receptor is mutated.

In one example, in the adjuvant treatment method provided by the first or second aspect above, the dominant negative TGF-β receptor lacks the intracellular signaling domain of TGF-β receptor.

In one example, in the adjuvant treatment method provided by the first or second aspect above, TGF-β type receptor is selected from type TGF-β RII receptor.

In one example, in the adjuvant treatment method provided by the first or second aspect above, the DN form of TGFβ receptor can comprise the extracellular domain of TGFβ receptor or its ligand binding part, and the intracellular domain is missing.

In one example, in the adjuvant treatment method provided by the first or second aspect above, the immune response inhibitor is a chimeric TGF-β receptor, which comprises an extracellular domain of TGFβ receptor and an intracellular domain of non TGFβ receptor; and the intracellular domain comprises the intracellular domain of immunostimulatory proteins or functional fragments thereof, which are from TLR1, TLR2, TLR3, TLR4, TLR5, TLR6, TLR7, TLR8, TLR9, CD28, 41BB, 41BB, OX40, CD3ζ, CD40, CD27, or a combination thereof.

In one example, in the adjuvant treatment method provided by the first or second aspect above, the extracellular domain of the chimeric TGF-β receptor comprises the extracellular domain of TGF-β receptor I and/or the extracellular domain of TGF-β receptor II; and the intracellular signaling domain of the receptor for IL-2 family proteins.

In one example, in the adjuvant treatment method provided by the first or second aspect above, the second polypeptide comprises: NKG2D, truncated NKG2D, extracellular domain of truncated NKG2D or its variants. In one example, the coexpressed polypeptide comprises antibody targeting NKG2D ligand or an antigen binding fragment thereof. In one example, the coexpressed polypeptide comprises TIGIT, truncated TIGIT, extracellular domain of truncated TIGIT or its variants, or SIRP-α, or its variants, or antibody targeting TIGIT ligand or an antigen binding fragment thereof.

In one example, in the adjuvant treatment method provided by the first or second aspect above, the method comprises administering immune cell therapy once, twice or more than three times.

In one example, in the adjuvant treatment method provided by the first or second aspect above, when administering immune cell therapy 2 or 3 times or more, the interval between adjacent immune cell therapy administrations should be at least about 28 days or more.

In one example, in the adjuvant treatment method provided by the first or second aspect above, pretreatment is administered about 1-12 days before each administration of the cell therapy.

In one example, in the adjuvant treatment method provided by the first or second aspect above, pretreatment is administered about 7 days before each administration of the cell therapy.

In one example, in the adjuvant treatment method provided by the first or second aspect above, pretreatment is administered about 5 days before each administration of the cell therapy.

In one example, in the adjuvant treatment method provided by the first or second aspect above, when administering immune cell therapy 2 or 3 times or more, the same or different pretreatment is administered before each administration of immune cell therapy.

In one example, in the adjuvant treatment method provided by the first or second aspect above, the pretreatment comprises the administration of a chemotherapy agent.

In one example, in the adjuvant treatment method provided by the first or second aspect above, the pretreatment comprises the administration of cyclophosphamide, fludarabine, albumin-bound paclitaxel, etoposide, cytarabine, methotrexate, vincristine, adriamycin, or any combinations thereof.

In one example, in the adjuvant treatment method provided by the first or second aspect above, the pretreatment comprises administering: cyclophosphamide; cyclophosphamide and fludarabine combination; or a combination of cyclophosphamide, fludarabine, and albumin-bound paclitaxel.

In one example, in the adjuvant treatment method provided by the first or second aspect above, the pretreatment is administered by intravenous infusion.

In one example, in the adjuvant treatment method provided by the first or second aspect above, wherein the fludarabine is administered at about 10-50 mg/m²/day, or about 15-40 mg/m²/day, or about 15-30 mg/m²/day, or about 20-30 mg/m²/day, or about 25, or 30 mg/m²/day; and the cyclophosphamide is administered at about 300-700 mg/m²/day, or about 400-650 mg/m²/day, or about 450-600 mg/m²/day, or about 450-550 mg/m²/day, or about 490-550 mg/m²/day, or about 250, 300, 350, 400, 450, or 500 mg/m²/day.

In one example, in the adjuvant treatment method provided by the first or second aspect above, wherein the fludarabine is administered at about 10-50 mg/m²/day, or about 15-40 mg/m²/day, or about 15-30 mg/m²/day, or about 20-30 mg/m²/day, or about 25, or 30 mg/m²/day; and the cyclophosphamide is administered at about 300-700 mg/m²/day, or about 400-650 mg/m²/day, or about 450-600 mg/m²/day, or about 450-550 mg/m²/day, or about 490-550 mg/m²/day, or about 250, 300, 350, 400, 450, or 500 mg/m²/day; and the albumin-bound paclitaxel is administered at not higher than about 300 mg/m²/day, or not higher than about 200 mg/m²/day, or not higher than about 150 mg/m²/day, or not higher than about 100 mg/m²/day, or not higher than about 80 mg/m²/day, or not higher than about 70, 69, 68, 67, 66, 65, 64, 63, 62, or 61 mg/m²/day, or the albumin-bound paclitaxel is administered at 100 mg/m²/day.

In one example, in the adjuvant treatment method provided by the first or second aspect above, wherein the pretreatment is administered daily for 2, 3, 4, 5, 6, or 7 days.

In one example, in the adjuvant treatment method provided by the first or second aspect above, wherein the cyclophosphamide and the fludarabine are administered daily for 2, 3, or 4 days, and the albumin-bound paclitaxel is administered once; or the cyclophosphamide, the fludarabine, and the albumin-bound paclitaxel are administered daily for 2, 3, or 4 days.

In one example, in the adjuvant treatment method provided by the first or second aspect above, wherein the cyclophosphamide and the fludarabine are administered daily for 2, 3, or 4 days, and the albumin-bound paclitaxel is administered once; or the cyclophosphamide, the fludarabine, and the albumin-bound paclitaxel are administered daily for 2, 3, or 4 days.

In one example, in the adjuvant treatment method provided by the first or second aspect above, wherein the fludarabine is administered at about 25 mg/m²/day for 3 days, and the cyclophosphamide is administered at about 300 mg/m²/day for 3 days.

In one example, in the adjuvant treatment method provided by the first or second aspect above, wherein the fludarabine is administered at about 25 mg/m²/day for 3 days, the cyclophosphamide is administered at about 300 mg/m²/day for 3 days, and the albumin-bound paclitaxel is administered at 100 mg/m²/day for one day.

In one example, in the adjuvant treatment method provided by the first or second aspect above, wherein the fludarabine is administered at about 30 mg/m²/day for 3 days, the cyclophosphamide is administered at about 250 mg/m²/day for 3 days, and the albumin-bound paclitaxel is administered at is 100 mg/m²/day for one day.

In one example, in the adjuvant treatment method provided by the first or second aspect above, immune cell therapy comprises no more than about 1×10¹² cells/time, 1×10¹¹ cells/time, 1×10¹⁰ cells/time, 5×10⁹ cells/time, 1×10⁹ cells/time, 9×10⁸ cells/time, 8×10⁸ cells/time, 7×10⁸ cells/time, 6×10⁸ cells/time, or 5×10⁸ cells/time.

In one example, in the adjuvant treatment method provided by the first or second aspect above, immune cell therapy comprises about 1×10⁸ cells/time, 2.5×10⁸ cells/time, 4×10⁸ cells/time, or 5×10⁸ cells/time.

In one example, in the adjuvant treatment method provided by the first or second aspect above, each administration of the immune cell therapy may be administered once or in multiple fractions.

In one example, in the adjuvant treatment method provided by the first or second aspect above, the imaging detection comprises: computed tomography (CT) scan, bone scan, magnetic resonance imaging (MRI), positron emission tomography (PET), ultrasonic detection, X-ray examination or a combination thereof.

In one example, in the adjuvant treatment method provided by the first or second aspect above, the tumor markers comprise: ALK, AFP, B2M, Beta-hCG, BTA, C-kit/cd117, CA15-3, CA19-9, CA724, CA-125, CA 27.29, Calcitonin, CEA, CD19, CD20, CD22, CD25, CD30, CD33, CGA, DCP, ER/PR, 5-HIAA, PSA, SMRP, a squamous cell carcinoma antigen (SCC), or a soluble fragment of cytokeratin 19 (CYFRA21-1).

In one example, in the adjuvant treatment method provided by the first or second aspect above, the levels of the tumor markers in the peripheral blood, body fluid, excreta, or tissue of the subject are detected by immunological, biological, or chemical techniques.

In one example, in the adjuvant treatment method provided by the first or second aspect above, the tumor antigens expressed by the solid tumors comprise: EGFR or a mutant thereof, B7H3, GPC3, Claudin 6, Claudin18.2, FAP, mesothelin, NKG2D ligand (NKG2DL), NKG2A, CD94, FCRH5, IL13Ru2, GD2, EpCAM, CEA, MUC1, MSLN, PSCA, AFP, or ERBB2.

In one example, in the adjuvant treatment method provided in the first or second aspect, the solid tumor comprises: liver cancer, pancreatic cancer, gastric cancer, esophageal cancer, gastroesophageal junction tumor, colon cancer, rectal cancer, small intestinal cancer, cholangiocarcinoma, gallbladder cancer, lung cancer, laryngeal cancer, kidney cancer, bladder cancer, ovarian cancer, breast cancer, uterine cancer, prostate cancer, gliomas, melanoma, neuroblastoma, sarcoma, or skin cancer.

In one example, in the adjuvant treatment method provided by the first or second aspect above, the method is used to treat subjects with liver cancer. After the local treatment or the local treatment combined with the other adjuvant treatment, CAR-T cells binding to GPC3, NKG2DL, EpCAM, and/or B7H3, or TCR-T cells binding to HBsAg or AFP are administered by intravenous infusion if no tumor recurrence and/or metastasis is detected by imaging technology.

In one example, in the adjuvant treatment method provided by the first or second aspect above, the method is used to treat subjects with GPC3-positive solid tumors. After the local treatment or the local treatment combined with the other adjuvant treatment, GPC3-CAR-T cells are administered by intravenous infusion if no tumor recurrence and/or metastasis is detected by imaging technology.

In one example, in the adjuvant treatment method provided by the first or second aspect above, the method is used to treat subjects with GPC3-positive solid tumors. After the local treatment or the local treatment combined with the other adjuvant treatment, GPC3-CAR-T cells are administered by intravenous infusion if the tumor marker level is detected to be higher than the upper limit of the normal range or higher than the baseline level, or the tumor marker level is progressively increased.

In one example, in the adjuvant treatment method provided by the first or second aspect above, the method is used to treat subjects with GPC3-positive solid tumors. After the local treatment or the local treatment combined with the other adjuvant treatment, GPC3-CAR-T cells are administered by intravenous infusion if the tumor marker level is detected to be higher than the upper limit of the normal range or higher than the baseline level, or the tumor marker level is progressively increased, and no tumor recurrence and/or metastasis is detected by imaging technology.

In one example, in the adjuvant treatment method provided by the first or second aspect above, the antigen binding unit comprised in the GPC3-CAR comprises a sequence having at least 90% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 62.

In one example, in the adjuvant treatment method provided by the first or second aspect above, the antigen binding unit comprised in the GPC3-CAR comprises HCDR1 shown in SEQ ID NO:31, HCDR2 shown in SEQ ID NO:32, HCDR3 shown in SEQ ID NO:33, LCDR1 shown in SEQ ID NO:34, LCDR2 shown in SEQ ID NO:35 and LCDR3 shown in SEQ ID NO:36; or HCDR1 shown in SEQ ID NO:17, HCDR2 shown in SEQ ID NO:18, HCDR3 shown in SEQ ID NO:19, LCDR1 shown in SEQ ID NO:20, LCDR2 shown in SEQ ID NO:21 and LCDR3 shown in SEQ ID NO:22; or HCDR1 shown in SEQ ID NO:17, HCDR2 shown in SEQ ID NO:18, HCDR3 shown in SEQ ID NO:19, LCDR1 shown in SEQ ID NO:25, LCDR2 shown in SEQ ID NO:21 and LCDR3 shown in SEQ ID NO:22.

In one example, in the adjuvant treatment method provided by the first or second aspect above, the antigen binding unit comprised in the GPC3-CAR comprises VH and VL, which respectively comprise sequences having at least 90% sequence identity with SEQ ID NO:37 and 38, or SEQ ID NO:23 and 24, or SEQ ID NO:26 and 27.

In one example, in the adjuvant treatment method provided by the first or second aspect above, the anti-GPC3-CAR comprises a sequence having at least 90% sequence identity with SEQ ID NO: 28, 29, 30, 60 or 61.

In one example, in the adjuvant treatment method provided by the first or second aspect above, the GPC3-positive solid tumors comprise: hepatocellular carcinoma, hepatic cell carcinoma, hepatoblastoma, rhabdomyosarcoma, malignant rhabdoid tumor, liposarcoma, Wilms tumor, York sac tumor, embryonic sarcoma of the liver, squamous cell carcinoma of the lung, Merkel cell carcinoma, lung cancer, breast cancer, colo-rectal cancer, or brain tumor.

In one example, in the adjuvant treatment method provided by the first or second aspect above, the antigen binding unit of the CLDN18.2-CAR comprises a sequence having at least 90% sequence identity with SEQ ID NO: 47, 48, 49, 50 or 63.

In one example, in the adjuvant treatment method provided by the first or second aspect above, the antigen binding unit comprised in the CLDN18.2-CAR comprises HCDR1 shown in SEQ ID NO:39, HCDR2 shown in SEQ ID NO:40, HCDR3 shown in SEQ ID NO:41, LCDR1 shown in SEQ ID NO:42, LCDR2 shown in SEQ ID NO:43 and LCDR3 shown in SEQ ID NO:44.

In one example, in the adjuvant treatment method provided by the first or second aspect above, the antigen binding unit comprised in the CLDN18.2-CAR comprises a sequence having at least 90% sequence identity with the VH shown in SEQ ID NO:45 and the VL shown in SEQ ID NO:46.

In one example, in the adjuvant treatment method provided by the first or second aspect above, the anti CLDN18.2-CAR comprises a sequence having at least 90% sequence identity with SEQ ID NO: 51, 52, 53, or 54.

In one example, in the adjuvant treatment method provided by the first or second aspect above, the CLDN18.2-positive solid tumor comprises: gastrointestinal tumor, gastric adenocarcinoma, gastroesophageal junction adenocarcinoma, gastric cancer, pancreatic cancer, gastroesophageal junction cancer, esophageal cancer, pancreatic ductal adenocarcinoma, or ovarian carcinoma.

In one example, in the adjuvant treatment method provided by the first or second aspect above, the CLDN18.2-positive solid tumor comprises: digestive system tumor.

In one example, in the adjuvant treatment method provided by the first or second aspect above, the subject has a high risk of cancer recurrence.

In one example, in the adjuvant treatment method provided by the first or second aspect above, the subject is human.

In one example, in the adjuvant treatment method provided by the first or second aspect above, the subject is resistant or refractory to the treatment before the administration of the method, or cannot be cured.

In one example, in the adjuvant treatment method provided by the first or second aspect above, the treatment before the administration of the method comprises: surgery, interventional treatment, or the other adjuvant treatment excluding immune cell therapy.

In one example, in the adjuvant treatment method provided by the first or second aspect above, the adjuvant treatment of surgical treatment or interventional treatment before the administration of the method does not comprise the immune cell therapy.

The present application also provides another method for treating solid tumors.

The present application provides a method for treating a subject with a tumor antigen-positive solid tumor, wherein the method comprises administering the subject an immune cell therapy comprising a chimeric receptor binding to the tumor antigen after treatment to reduce or eliminate the tumor burden.

In one example, the tumor burden comprises tumor lesions detected by imaging.

In one example, after local treatment, no tumor lesion is detected by imaging detection before the immune cell therapy is administered.

In one example, after local treatment, no enlargement of other small tumor lesions without the local treatment is detected by imaging detection before the immune cell therapy is administered.

In one example, after local treatment, no new tumor lesions were detected by imaging detection before the immune cell therapy is administered.

In one example, after local treatment, the tumor marker level is detected to be higher than the upper limit of the normal range or the tumor marker level is progressively increased before the immune cell therapy is administered.

In one example, after local treatment, the tumor marker level is detected to be higher than the upper limit of the normal range, or the tumor marker level is progressively increased, but no new tumor lesions are detected by imaging detection before the immune cell therapy is administered.

In one example, after local treatment, the tumor marker level is detected to be higher than the upper limit of the normal range, or the tumor marker level is progressively increased, and no enlargement of other small tumor lesions without the local treatment is detected by imaging detection before the immune cell therapy is administered.

In one example, after local treatment, the other adjuvant treatment other than immune cell therapy is administered before and/or after administration of the immune cell therapy.

In one example, the subject cannot tolerate the other adjuvant treatment other than immune cell therapy before the immune cell therapy is administered.

In one example, the other adjuvant treatment comprises: chemotherapy, radiotherapy, hormone therapy, immune checkpoint inhibitor therapy, immunomodulator therapy, antibody therapy, antiangiogenic drug therapy, small molecule compound therapy, or any combination thereof; preferably, the antibody treatment comprises: administration of monoclonal antibody, antibody-drug conjugate, bifunctional antibody, or multifunctional antibody.

In one example, the other adjuvant treatment other than immune cell therapy is administered after local treatment, and then the immune cell therapy is administered after detecting that the tumor marker level is higher than the upper limit of the normal range, or the tumor marker level is progressively increased.

In one example, the other adjuvant treatment other than the immune cell therapy is administered after local treatment, and then the immune cell therapy is administered after no enlargement of tumor lesion is detected.

In one example, the other adjuvant treatment other than the immune cell therapy is administered after local treatment, and then the immune cell therapy is administered after a new tumor lesion is detected.

In one example, the method comprises local treatment of tumor lesions after administering the subject the immune cell therapy.

In one example, the local treatment is tumor surgical treatment, microwave treatment, vascular embolization, gamma knife treatment or a combination thereof.

In one example, about 12 months, 11 months, 10 months, 9 months, 8 months, 7 months, 6 months, 5 months, 4 months, 3 months, 2 months, 60 days, 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after the treatment to reduce or eliminate tumor burden, the immune cell therapy is administered.

In the second aspect, the present application provides a method for treating a subject with GPC3-positive liver cancer, which comprises administering the subject immune cells comprising GPC3-CAR after the treatment to reduce or eliminate tumor burden.

In the third aspect, the present application provides a method for treating a subject with a tumor antigen-positive solid tumor, which comprises administering an immune cell therapy comprising a chimeric receptor binding to the tumor antigen after surgery.

In one example, after the method is administered to the subject, the number of circulating tumor cells (CTCs) of the subject is reduced, and/or tiny tumor lesions are eliminated.

In one example, after the method is administered to the subject, the disease-free survival and/or overall survival of the subject are prolonged.

In the fourth aspect, the present application provides the use of a composition for preparing a medicament for adjuvant treatment of a subject with a tumor antigen-positive solid tumor by intravenous infusion after surgery. The composition comprises a therapeutically effective amount of immune cells comprising a chimeric receptor that binds to the tumor antigen.

In one example, the composition further comprises: hormones, immune checkpoint inhibitors, immune modulators, antibodies, anti angiogenic drugs, small molecule compounds, or a combination thereof; preferably, the composition also comprises: monoclonal antibody, antibody-drug conjugate, bifunctional antibody or multifunctional antibody.

In one example, the composition also comprises: (a) one or more inhibitors of checkpoint molecules, comprising PD-1, PDL-1, TIM-3, TIGIT, LAG-3, CTLA-4, 2B4, 4-1BB, 4-1BBL, A2aR, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, RARA (retinoic acid receptor alpha), TLR3, VISTA, NKG2A/HLA-E, or inhibitory KIR;
(b) one or more of atezolizumab, avelumab, durvalumab, ipilimumab, IPH4102, IPH43, IPH33, lirimumab, monalizumab, nivolumab, pembrolizumab, and derivatives or functional equivalents thereof; or
(c) at least one of atezolizumab, nivolumab, and pembrolizumab; or
(d) one or more of venetoclax, azacitidine, and pomalidomide; or AVASTIN.

In one example, the composition is administered to a subject one or more times.

In the fifth aspect, the present application provides a method for prolonging the survival of a subject with a tumor antigen-positive solid tumor. The method comprises administering an immune cell therapy comprising a chimeric receptor binding to the tumor antigen to the subject after local treatment to reduce or eliminate the tumor burden.

In the sixth aspect, the present application provides a method for prolonging the disease-free survival of a subject with a tumor antigen-positive solid tumor. The method comprises administering an immune cell therapy comprising a chimeric receptor binding to the tumor antigen to the subject after local treatment to reduce or eliminate the tumor burden.

In the seventh aspect, the present application provides a method for reducing tumor recurrence of a subject with a tumor antigen-positive solid tumor after local treatment to reduce or eliminate tumor burden. The method comprises administering an immune cell therapy comprising a chimeric receptor binding to the tumor antigen to the subject after local treatment to reduce or eliminate tumor burden.

In the eighth aspect, the present application provides a method for reducing tumor metastasis of a subject with a tumor antigen-positive solid tumor after local treatment to reduce or eliminate tumor burden. The method comprises administering an immune cell therapy comprising a chimeric receptor binding to the tumor antigen to the subject after local treatment to reduce or eliminate tumor burden.

In the ninth aspect, the present application provides a method for reducing tumor recurrence of a subject with a tumor antigen-positive solid tumor after tumor surgical treatment. The method comprises administering an immune cell therapy comprising a chimeric recipient binding to the tumor antigen to the subject after tumor surgical treatment.

In the tenth aspect, the present application provides a method for reducing tumor metastasis of a subject with a tumor antigen-positive solid tumor after tumor surgical treatment. The method comprises administering an immune cell therapy comprising a chimeric receptor binding to the tumor antigen to the subject after tumor surgical treatment.

In the eleventh aspect, the present application provides a method for treating a subject with a tumor antigen-positive solid tumor and without an indication for tumor surgical treatment. The method comprises administering an immune cell therapy comprising a chimeric receptor binding to the tumor antigen to the subject after local treatment to reduce or eliminate tumor lesions.

In the twelfth aspect, the present application provides a method for reducing the number of circulating tumor cells (CTCs) in the body of a subject with a tumor antigen-positive solid tumor and/or reducing small tumor lesions in the body. The method comprises administering an immune cell therapy comprising a chimeric receptor binding to the tumor antigen to the subject after local treatment to reduce or eliminate the tumor lesions.

In the thirteenth aspect, the present application provides a method for reducing the number of circulating tumor cells and/or reducing small tumor lesions in the body of a subject who has received local treatment to reduce or eliminate tumor lesions. The method comprises administering an immune cell therapy to the subject comprising a chimeric receptor binding to the tumor antigen.

It should be understood that within the scope of the present application, the above technical features of the present application and the technical features specifically described below (such as Examples) can be combined with each other to form a new or preferred technical solution. Due to space limitations, it will not repeat them here.

### BRIEF DESCRIPTION OF THE DRAWINGS

FIG. 1: Overview of disease course and treatment timeline of patient 1 FIG. 2: MRI images showed intrahepatic tumor complication of inferior vena cava tumor thrombus(April 2015; arrows) FIG. 3: MRI images before administration of CAR-GPC3 T-cells. The patient was treated with MWA and GKRS for these lesions (arrows). No active lesions were shown on T2, T1, and T1 contrast-enhanced images at the baseline before the patient received CAR-GPC3 T-cell infusion (July 2015).

FIG. 4: DNA was extracted from peripheral blood, and the CAR-GPC3 transgene copies per microgram of DNA were evaluated by quantitative PCR assay (left y-axis). AFP level was also monitored (right y-axis). Dose-schedule of each CAR-GPC3 T-cell infusion is listed below.

FIG. 5: No recurrence of tumor lesion after administration of CAR-GPC3 T-cells in patient 1. Liver MRI images in (A) week 3 after the initial infusion and (B) month 4 after the initial infusion. The arrows represent the necrotic tumor after the treatment.

FIG. 6: Liver MRI images during the follow-up in patient 1 after CAR-GPC3 T-cell treatment. There was no active focus shown in the images: month 12 (August 2016), month 22 (June 2017), month 35 (July 2018), month 45 (May 2019), month 57 (May 2020), and month 70 (June 2021). The arrows represent the necrotic tumor after the treatment.

FIG. 7: Overview of disease course and treatment timeline of patient 2.

FIG. 8: MRI scan of vascular invasion before enrollment. Inferior vena cava filling defect was shown on T1 contrast-enhanced image, indicating tumor thrombus (July 2015; arrows) and non-active scar tissue after GKRS treatment (September 2015; arrows).

FIG. 9: MRI imaging on retroperitoneal lymphatic metastasis before and after CAR-GPC3 T-cell infusions (arrows).

FIG. 10: Dynamic changes in the retroperitoneal lymphatic metastasis after CAR-GPC3 T-cell infusions. The lesion changes are presented as a percentage based on the baseline value. The line with triangles represents the changes in the short axis, and the line with circles represents the changes in the long axis. The patient remained in SD per RECIST criteria.

FIG. 11: Reduction of tumor biomarker AFP after CAR-GPC3 T-cell infusions. Cycle 1 included six split infusions with a total dose of 40.7×10⁸ CAR-GPC3 T-cells; Cycle 2 included one infusion with 11.1×10⁸ CAR-GPC3 T-cells.

FIG. 12: The MRI of inferior vena cava tumor thrombus during the follow-up in patient 2 after CAR-GPC3 T-cell treatment. There was no recurrence in the images: month 8 (September 2016), month 20 (September 2017), month 30 (July 2018), month 44 (September 2019), month 57 (October 2020), and month 70 (November 2021). The arrows represent the inferior vena cava tumor thrombus after the treatment.

FIG. 13: Liver MRI images during the follow-up in patient 2 after CAR-GPC3 T-cell treatment. There was no active focus shown in the images: month 8 (September 2016), month 20 (September 2017), month 30 (July 2018), month 44 (September 2019), month 57 (October 2020), and month 70 (November 2021). The arrows represent the necrotic tumor after the treatment.

FIG. 14: The changes of CA19-9 expression levels detected during the treatment.

FIG. 15: the changes of CAR copy number and IL6 expression level in vivo; and the day of CAR-T cell infusion is set as D0.

### DETAIL DESCRIPTION OF THE INVENTION

The CAR provided in the present application is not limited to the CAR with specific amino acid sequences as shown in SEQ ID NO: 28, 29, 30, 51, 52, 53, 54, 60 and/or 61, the CAR with amino acid sequences that have been modified, and/or replaced, and/or deleted, and/or added with one or more amino acids and have more than 60%, 65%, 70%, 75%, 80%, 85%, 90%, or 95% identity with the amino acid sequences shown in SEQ ID NO: 28, 29, 30, 51, 52, 53, 54, 60, and/or 61 and have the same function are also within the protection scope of the present application.

Unless specifically defined, all technical and scientific terms used herein have the same meaning commonly understood by technicians in the fields of gene therapy, biochemistry, genetics and molecular biology. All methods and materials similar or equivalent to those described herein can be used in the practice or test of the present application, among which, the suitable methods and materials described herein. All publications, patent applications, patents and other references mentioned herein are incorporated herein by reference in their entirety. In case of conflict, this specification, including definitions, shall prevail. In addition, unless otherwise specified, the materials, methods, and Examples of the present application are illustrative only and are not intended to be restrictive. According to the content of the present application, those skilled in the art should understand that many changes or changes can be made in the disclosed specific embodiment, and the same or similar results are still obtained without departing from the spirit and scope of the present application. The scope of the present application is not limited to the specific embodiment described herein (which is only expected to be an example of various aspects of the present application), and functionally equivalent methods and components are within the scope of the present application. The present application comprises the variant and modification of the subject matter of the present application for various purposes and conditions.

Unless otherwise specified, the practice of the present application will adopt the traditional technologies of cell biology, cell culture, molecular biology, transgenic biology, microbiology, recombinant DNA and immunology, which belong to the technical scope of the art. These techniques are fully explained in the literature.

In the present application, the description of the scope form is only for convenience and brevity, and should not be regarded as an unalterable limitation of the scope of the present application. Therefore, the description of the range should be considered to specifically disclose all possible sub ranges and individual values within that range.

### Terms

The term “about” refers to the general error range of each value that is easily known by those skilled in the art. The “about” value or parameter described herein comprises embodiments that point to the value or parameter itself. For example, the description of “about X” comprises the description of “X”. Herein, “about” can be an acceptable error range in the technical field. For example, it may refer to a value or parameter within ±10% of the “about” value or parameter, for example, about 5 um may comprise any number between 4.5 um and 5.5 um.

The term “treatment” refers to alleviating or reducing the severity of at least one symptom or indication, to temporarily or permanently eliminate the cause of symptoms, to delay or inhibit tumor growth, to reduce tumor cell burden or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to improve the duration of survival of the subject. In many examples, the terms “tumor”, “lesion”, “tumor lesion”, “cancer” and “malignant disease” are used interchangeably and refer to one or more types of tumors.

The term “local treatment” refers to the method for treating the local part of the tumor, including tumor surgical treatment, interventional treatment (for example, radiotherapy, endoscopic treatment, high-intensity focused ultrasound treatment). In one example, local treatment comprises: surgical treatment, cryoablation, microwave treatment, vascular embolization, radiofrequency treatment, gamma knife treatment, focused ultrasound treatment, photodynamic therapy, argon-helium knife treatment, radioactive particle implantation or any combination thereof. For example, microwave ablation (MWA) is used to treat multiple small or minimal lesions of tumors. For example, gamma knife radiotherapy (GKRS) is used to treat inferior vena cava tumor thrombus (IVCTT). For example, transcatheter arterial chemoembolization (TACE) is used to treat multiple small or minimal lesions of tumors.

The term “radical tumor surgery” refers to the removal of the tumor mass while expanding the scope of resection to remove suspicious tissues and lymph nodes around the mass, in order to achieve the purpose of “radical cure”. In one example, the pathological evaluation of the surgical resection specimen shows a negative margin. For example, it comprises radical resection of liver cancer, radical resection of pancreatic cancer, radical pancreatoduodenectomy, etc.

The term “adjuvant treatment” refers to the treatment administered before or after the local treatment that reduces or eliminates the local lesions of the tumor, so as to reduce the risk of disease recurrence after the local treatment. The goal of the adjuvant treatment is to prevent cancer recurrence, thereby reducing cancer-related deaths. For example, the adjuvant treatment comprising the immune cell therapy described in the present application may be administered after local treatment to eliminate the remaining cancer cells. For example, the immune cells can target tumor cells expressing the tumor antigen. For example, the immune cells can target tumor cells expressing the tumor antigen on the cell membrane. For example, it comprises administering the immune cell therapy once, twice, three times or more. For example, when the immune cell therapies are administered twice, three times or more, the chimeric receptors comprised in the immune cells bind different tumor antigens. For example, when the immune cell therapies are administered twice, three times or more, the chimeric receptors comprised in the immune cells bind to the same tumor antigen.

For example, the adjuvant treatment comprises administering at least one or more TCR-T cell treatments after local treatment.

In one example, the adjuvant treatment comprises administering CAR-T cells by intravenous infusion once, twice, three times or more after local interventional treatment. In one example, the adjuvant treatment comprises administering CAR-T cells by intravenous infusion once, twice, three times or more after GKRS treatment of IVCTT. In one example, the adjuvant treatment comprises administering CAR-T cells by intravenous infusion once, twice, three times or more after MWA treatment of multiple small or minimal lesions of tumors (e.g., liver cancer).

In one example, the diameter of small tumor lesions is less than 5 cm, 4 cm, or 3 cm, or the sum of the diameters of cancer lesions is less than 5 cm, 4 cm, or 3 cm. In one example, the diameter of minimal tumor lesions is less than 2 cm or 1 cm, or the sum of the diameters of cancer lesions is less than 2 cm or 1 cm.

In one example, the adjuvant treatment comprises administering CAR-T cells by intravenous infusion once, twice, three times or more after radical tumor surgery. For example, when the immune cell therapies are administered twice, three times or more, the chimeric receptors comprised in the immune cells bind to the same or different tumor antigens.

The term “the other adjuvant treatment” refers to the adjuvant treatment that does not comprise the immune cell therapy. For example, the other adjuvant treatment comprises chemotherapy, radiotherapy, hormone therapy, immune checkpoint inhibitor therapy, immune modulator therapy, antibody therapy, anti angiogenic drug therapy, small molecule compound therapy, or a combination thereof. For example, antibody therapy comprises: administration of monoclonal antibodies, antibody-drug conjugate therapy, bifunctional antibodies or multifunctional antibodies. For example, the other adjuvant treatment comprises systemic chemotherapy. For example, systemic chemotherapy comprises administration by oral or intravenous administration. For example, the other adjuvant treatment comprises: (a) one or more antagonists of checkpoint molecules, comprising PD-1, PDL-1, TIM-3, TIGIT, LAG-3, CTLA-4, 2B4, 4-1BB, 4-1BBL, A2aR, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR;
(b) One or more of atezolizumab, avelumab, durvalumab, ipilimumab, IPH4102, IPH43, IPH33, lirimumab, monalizumab, nivolumab, pembrolizumab, and derivatives or functional equivalents thereof; or (c) at least one of atezolizumab, nivolumab, and pembrolizumab; or (d) the therapeutic agent comprises one or more of venetoclax, azacitidine, and pomalidomide.

In one example, after local tumor treatment, the immune cell therapy is administered once, twice, three times or more. For example, after the local tumor treatment combined with the other adjuvant treatment, the immune cell therapy is administered once, twice, three times or more. For example, after the local tumor treatment, the immune cell therapy is administered once, twice, three times or more, followed by the other adjuvant treatment.

In one example, after local interventional treatment of tumor, the immune cell therapy is administered by intravenous infusion once, twice, three times or more. For example, after local interventional treatment of tumor combined with the other adjuvant treatment, the immune cell therapy is administered by intravenous infusion once, twice, three times or more. For example, after local interventional treatment of tumor, the immune cell therapy is administered by intravenous infusion once, twice, three times or more, followed by the other adjuvant treatment.

In one example, after tumor surgical treatment, the immune cell therapy is administered by intravenous infusion once, twice, three times or more. For example, after tumor surgical treatment combined with the other adjuvant treatment, the immune cell therapy is administered by intravenous infusion once, twice, three times or more. For example, after tumor surgical treatment, the immune cell therapy is administered by intravenous infusion once, twice, three times or more, followed by the other adjuvant treatment.

In one example, after the radical tumor surgical treatment, the immune cell therapy is administered by intravenous infusion once, twice, three times or more. For example, after radical tumor surgical treatment combined with the other adjuvant treatment, the immune cell therapy is administered by intravenous infusion once, twice, three times or more. For example, after the radical tumor surgical treatment, the immune cell therapy is administered by intravenous infusion once, twice, three times or more, followed by the other adjuvant treatment.

The term “imaging recurrence and/or metastasis” refers to recurrent tumor lesions or new tumor metastatic lesions detected by imaging technology. For example, imaging technology, including: computed tomography (CT) scan, bone scan, magnetic resonance imaging (MRI), positron emission tomography (PET), ultrasonic testing, X-ray examination or a combination thereof.

The term “recurrence” refers to the frequent or repeated diagnosis of solid tumors (for example, liver cancer, pancreatic cancer, gastric cancer, gastroesophageal junction tumor, lung cancer or head and neck cancer) suffered by tumor patients, or the frequent or repeated occurrence of tumors suffered by patients, such as primary tumors and/or new tumors that may represent a recurrence of a previous tumor. For example, the immune cell therapy is administered to inhibit the recurrence of tumors in patients receiving the local tumor treatments. For example, the immune cell therapy is administered to inhibit the recurrence of liver cancer, pancreatic cancer, gastric cancer, gastroesophageal junction tumor, lung cancer or head and neck cancer tumor in patients receiving the local tumor treatment.

In one example, tumor recurrence and/or metastasis refers to the tumor recurrence or metastasis as indicated by imaging, histology, and/or cytopathology.

In one example, the “no imaging recurrence/metastasis” refers to that tumor recurrence and/or metastasis are not detected by imaging technology. For example, the imaging technology comprises computed tomography (CT) scan, bone scan, magnetic resonance imaging (MRI), positron emission tomography (PET), ultrasonic testing, X-ray examination, or a combination thereof.

The term “minimal residual disease (MRD)” refers to the state that tumor cells still exist in the body of patients with solid tumors after treatment, despite the residual lesions are not detected by traditional imaging techniques (including PET/X-ray, CT, MRI, etc.) or laboratory methods (such as observing cells under a microscope and/or tracking tumor markers in the blood). MRD is an important factor causing tumor recurrence, representing the possibility of tumor persistence or possible clinical progression. MRD in solid tumors is also known as Molecular Residual Disease, that is, molecular residual lesions, which can be detected by ctDNA (circulating tumor DNA) or CTC (circulating tumor cells) in peripheral blood. For example, ctDNA or CTC were detected in peripheral blood before the administration of the immune cell therapy. For example, ctDNA or CTC could not be detected in peripheral blood after the administration of the immune cell therapy. For example, after the administration of the immune cell therapy, the amount of ctDNA or CTC in peripheral blood decreases compared with that before the immune cell therapy.

The term “effective amount” or “therapeutic effective amount” refers to a dose sufficient to treat an individual tumor. It depends on the stage and severity of the treated tumor, the age, weight and general health of the subject, and the judgment of the prescribing doctor. The administrations of the immune cell therapy once, twice, three times or more may be required according to the judgment of the prescribing doctor or those skilled in the art. For example, it refers to the dose of CAR-T cells administered as the adjuvant treatment after surgical resection of solid tumors. For example, it refers to the dose of CAR-T cells administered as the adjuvant treatment after interventional treatment of local lesions of solid tumors.

An example comprises multiple administrations of the immune cell therapy, and the dose of each administration of the immune cell therapy may be the same or different. For example, the one-time the immune cell therapy described in the present application refers to a course of treatment, or a single course of treatment. For example, the multiple immune cell therapy described in the present application refers to multiple courses of treatment.

In one example, compared with untreated subjects, or subjects who are only administered local treatment (e.g., interventional treatment or surgical treatment), or subjects who are administered local treatment (e.g., interventional treatment or surgical treatment) combined with the other adjuvant treatment, after local treatment, the administration of adjuvant treatment comprising CAR-T cells results in one or more of the following: (a) inhibiting tumor growth, or improving tumor necrosis, tumor shrinkage, and/or tumor disappearance; (b) reducing the severity or duration of tumor symptoms or indications (such as tumor lesions); (c) delaying tumor growth and occurrence; (d) inhibiting tumor metastasis; (e) preventing tumor growth and recurrence; (f) improving the survival of subjects with tumors; and/or (g) delaying surgery.

The term “solid tumor” usually refers to an abnormal tissue mass that does not comprise cysts or fluid areas. Solid tumors can be benign (not cancer) or malignant (cancer). Solid tumors in the present application refer to malignant solid tumors, including different types of solid tumors named after the cell types that formed them, namely sarcoma, carcinoma and blastoma. Non limiting examples of solid tumors comprise: ovarian cancer, breast cancer, bladder cancer, brain cancer, colon cancer, intestinal cancer, liver cancer, lung cancer, pancreatic cancer, prostate cancer, skin cancer, gastric cancer, glioblastoma, laryngeal cancer, melanoma, neuroblastoma, adenocarcinoma, glioma, soft tissue sarcoma and various cancers (including prostate cancer and small cell lung cancer), astrocytoma, fibrosarcoma, myxosarcoma, liposarcoma, oligodendroglioma, ependymoma, medulloblastoma, primitive neuroectodermal tumor (PNET), chondrosarcoma, osteogenic sarcoma, pancreatic ductal adenocarcinoma, small and large cell lung adenocarcinoma, chordoma, vascular sarcoma, endothelial sarcoma, squamous cell carcinoma, bronchoalveolar carcinoma, epithelial adenocarcinoma and its liver metastasis, lymphangiosarcoma, cholangiocarcinoma, gallbladder cancer, synovial tumor, mesothelioma, Ewing's tumor, rhabdomyosarcoma, basal cell carcinoma, bronchial carcinoma, renal cell carcinoma, choriocarcinoma, seminoma, embryonal carcinoma, Wilms' tumor, testicular tumor, medulloblastoma, craniopharyngioma, ependymoma, pineal gland tumor, hemangioblastoma, acoustic neuroma, oligodendroglioma, meningioma, neuroblastoma, retinoblastoma, multiple myeloma, Waldenstrom's macroglobulinemia and heavy chain disease, breast tumors such as ductal and lobular adenocarcinoma, cervical cancer, ovarian cancer, bladder cancer. For example, solid tumors comprise but are not limited to: liver cancer, pancreatic cancer, gastric cancer, esophageal cancer, esophageal gastric junction tumor, colon cancer, rectal cancer, small intestinal cancer, cholangiocarcinoma, gallbladder cancer, lung cancer, laryngeal cancer, kidney cancer, bladder cancer, ovarian cancer, breast cancer, uterine cancer, prostate cancer, gliomas, melanoma, neuroblastoma, sarcoma, or skin cancer.

The efficacy evaluation of the method provided by the present application comprises the treatment response according to the Response Evaluation Criteria In Solid Tumors (RECIST) or other similar criteria. RECIST is a set of criteria for evaluating progression, stability or responsiveness of tumors and/or cancer cells jointly developed by the National Cancer Institute of the United States, the clinical trials group of the National Cancer Institute of Canada and the European Organization for Research and Treatment of Cancer. RECIST defines tumor size as the sum of the longest diameter (SLD) of target lesion, and classifies patients into complete response (CR), partial response (PR), stable disease (SD) and progression disease(PD). CR and PR represent no new tumor lesions, 100% reduction and more than 30% reduction, respectively; PD represents a 20% increase in the tumor size from the observed minimum size or appearance of new lesions. For example, the method efficacy evaluation provided by the present application refers to RECIST. For example, the method efficacy evaluation provided by the present application refers to RECIST v1.1.

The term “host” or “subject” refers to a recipient receiving a graft, which can be human or non-human mammals, such as dogs, cats, cattle, horses, mice, rats, rabbits, or their transgenic species. For example, it can be an individual who receives implantation of exogenous cells, such as human. For example, “subjects” can be clinical patients, clinical trial volunteers, laboratory animals, etc. The subject may be suspected of having a disease characterized by cell proliferation or having a disease characterized by cell proliferation and be diagnosed with a disease characterized by cell proliferation. For example, the subject is suffering from or may suffer from immune diseases such as autoimmune diseases, or suffering from inflammatory diseases. For example, the subject has a solid tumor. For example, the subject is human with a solid tumor.

In one example, the subject has a high risk of cancer recurrence. For example, the subject with the solid tumor has a high risk of cancer recurrence. The “subject” and “patient” in the present application can be used interchangeably. For example, the subjects include subjects with primary tumors, established tumors, or recurrent tumors (advanced malignancies). For example, the subjects include human subjects who have liver cancer, pancreatic cancer, gastric cancer, gastroesophageal junction tumor, lung cancer or head and neck cancer and/or requiring treatment for liver cancer, pancreatic cancer, gastric cancer, gastroesophageal junction tumor, lung cancer or head and neck cancer. For example, the subjects include patients with solid tumors who are resistant to or refractory to previous treatments (e.g., surgery, interventional treatment, and/or the other adjuvant treatments excluding the immune cell therapy) or are not cured or the disease is not controlled by the previous treatments. For example, the subjects include those with liver cancer, pancreatic cancer, gastric cancer, gastroesophageal junction tumor, lung cancer or head and neck cancer who are candidates for curative surgery.

The term “tumor burden” include volume size or differentiation degree of tumor, or type or stage of metastasis, and/or appearance and disappearance of common complications of middle-advanced cancer, such as cancerous hydrothorax and ascites, and/or changes in appearance or level of tumor markers. For example, the size of the tumor is measured by a ruler of PET (positron emission computed tomography). For example, the size of the tumor is measured by a ruler of CT (computed X-ray tomography). For example, the size of the tumor is measured by a ruler of magnetic resonance imaging (MRI).

The term “tumor marker”, also known as tumor label, refers to substances that characteristically present in malignant tumor cells, or are abnormally produced by the malignant tumor cells, or are produced by a host's response to tumor stimulation, and can reflect the occurrence and development of tumors and monitor the response of tumors to treatment. The tumor markers exist in tissues, body fluids and excretions of tumor patients and can be detected by immunological, biological and chemical methods. The tumor markers are used in tumor screening, diagnosis, efficacy monitoring and prognosis prediction. After tumor treatment, the level of the tumor markers in vivo is closely related to imaging recurrence. Patients with a trend of increased levels of the tumor markers have a high probability of subsequent development of imaging recurrence. Patients with persistently elevated levels of the tumor markers have a shorter time to imaging recurrence. For example, the levels of the tumor markers in peripheral blood are detected. For example, the levels of the tumor markers in body fluids or excretions of tumor patients are detected. For example, the levels of the tumor markers in tissues of tumor patient are detected.

In one example, the tumor markers comprise, ALK, AFP, B2M, Beta-hCG, BTA, C-kit/cd117, CA15-3, CA19-9 (CA199), CA724, CA-125 (CA125), CA 27.29, calcitonin, CEA, CD19, CD20, CD22, CD25, CD30, CD33, CgA, DCP, ER/PR, 5-HIAA, PSA, SMRP, a squamous cell carcinoma antigen (SCC), or a soluble fragment of cytokeratin 19 (CYFRA21-1).

In one example, after local treatment, the level of the tumor marker decreases compared with that before the local treatment. For example, after the local treatment and before administration of adjuvant treatments, the level of the tumor marker detected is defined as a baseline level. For example, the baseline level is above the upper limit of a normal range. For example, the baseline level is in the normal range. The progressively increase of the level of the tumor marker refers to an increase of the level of the tumor marker by at least 20% within any 3 months after the local treatment; or, after the local treatment, an increase of any two consecutive tests is greater than or equal to 10% compared with the previous test.

In one example, after the local treatment, the level of the tumor marker decreases to the normal range, and ctDNA is detected in peripheral blood, then the immune cell therapy is administered. For example, after the local treatment, if there is no imaging recurrence and/or metastasis, and ctDNA is detected in the peripheral blood, the immune cell therapy is administered.

In one example, after the local treatment, the level of the tumor marker first decreases to the normal range; when the level of the tumor marker begins to rise above the upper limit of the normal range, the immune cell therapy is administered. For example, after the local treatment, the level of the tumor marker first decreases to the normal range; when the level of the tumor marker begins to rise above the upper limit of the normal range and increases progressively, the immune cell therapy is administered. For example, after the local treatment, the level of the tumor marker first decreases but remains is higher than the upper limit of the normal range, then the immune cell therapy is administered. For example, after the local treatment, the level of the tumor marker first decreases but remains is higher than the upper limit of the normal range; when the level of the tumor marker begins to rise and increases progressively, the immune cell therapy is administered. In the above example, there is no imaging recurrence and/or metastasis during the period after the local treatment and before the administration of the immune cell therapy. In the above example, during the period after the local treatment and before the administration of the immune cell therapy, there is no imaging recurrence and/or metastasis, and no enlargement of the other minimal tumor lesions without the local treatment by imaging.

The present application provides a method for treating patients with solid tumors that are positive for tumor antigen. For example, when ctDNA or CTC is detected in peripheral blood after the local treatment for tumor reduction or resection, or after the local treatment combined with the other adjuvant treatment, immune cells (CAR-T, TCR-T) are administered by intravenous infusion.

For example, after the local treatment for tumor reduction or resection, or after the local treatment combined with the other adjuvant treatment, when the tumor marker level is higher than the upper limit of the normal range, immune cells (CAR-T, TCR-T) are administered by intravenous infusion.

For example, after the local treatment for tumor reduction or resection, or after the local treatment combined with the other adjuvant treatment, when the tumor marker level is higher than the upper limit of the normal range, and there is no imaging recurrence and/or metastasis, immune cells (for example, CAR-T, TCR-T) are administered by intravenous infusion. For example, after the local treatment for tumor reduction or resection, or after the local treatment combined with the other adjuvant treatment, when the tumor marker level is higher than the upper limit of the normal range, there is no imaging recurrence and/or metastasis, and no enlargement of the other minimal tumor lesions without the local treatment is detected by imaging, immune cells (for example, CAR-T, TCR-T) are administered by intravenous infusion. For example, after the local treatment for tumor reduction or resection, or after the local treatment combined with the other adjuvant treatment, when the tumor marker level is higher than the upper limit of the normal range and increases progressively, immune cells (for example, CAR-T, TCR-T) are administered by intravenous infusion. For example, after the local treatment for tumor reduction or resection, or after the local treatment combined with the other adjuvant treatment, when the tumor marker level is higher than the upper limit of the normal range and increases progressively, there is no imaging recurrence and/or metastasis, immune cells (for example, CAR-T, TCR-T) are administered by intravenous infusion. For example, after the local treatment for tumor reduction or resection, or after the local treatment combined with the other adjuvant treatment, when the tumor marker level is higher than the upper limit of the normal range and increases progressively, there is no imaging recurrence and/or metastasis, and no enlargement of the other minimal tumor lesions without the local treatment is detected by imaging, immune cells (for example, CAR-T, TCR-T) are administered by intravenous infusion. For example, after the local treatment for tumor reduction or resection, or after the local treatment combined with the other adjuvant treatment, there is no imaging recurrence and/or metastasis, immune cells (e.g., CAR-T, TCR-T) are administered by intravenous infusion. For example, after the local treatment for tumor reduction or resection, or after the local treatment combined with the other adjuvant treatment, if there is no imaging recurrence and/or metastasis, and no enlargement of the other minimal tumor lesions without the local treatment be detected by imaging, immune cells (for example, CAR-T, TCR-T) are administered by intravenous infusion. For example, the solid tumors include gastric cancer and gastroesophageal junction tumors, pancreatic cancer, liver cancer, etc. For example, the local treatment includes: surgical treatment (e.g., radical surgery), interventional treatment (e.g., MWA, GKRS, TACE, etc.). For example, the tumor antigens of pancreatic cancer comprises MSLN, MUC1, HER2, CEA, PSCA, CLDN18.2, EpCAM, B7H3, or NKG2DL. For example, the tumor antigens of gastric cancer or gastroesophageal cancer comprises CLDN18.2, EpCAM, B7H3, or NKG2DL. For example, the tumor antigens of liver cancer comprises GPC3, NKG2DL, HBsAg, EpCAM, B7H3, AFP. For example, immune cells that bind to MSLN, MUC1, ERBB2, CEA, PSCA, CLDN18.2, EpCAM, B7H3, NKG2DL, or GPC3 are CAR-T cells. For example, immune cells that bind to HBsAg or AFP are TCR-T cells.

The term “Genome editing/Gene editing” refers to the genetic engineering technology that uses site-specific nucleases to insert, knock out, modify or replace DNA at a specific position in the genome of an organism to change the DNA sequence. Gene editing can be used to achieve precise and efficient gene knockout or gene knock in. Nucleases are used for gene knockout or gene knock in, including CRISPR/Cas technology, ZFN technology, TALEN technology and TALEN-CRISPR/Cas technology, Base Editor technology, Prime Editor technology and Meganuclease technology.

In one example, CRISPR technology is used to construct endogenous TCR/B2M, TCR/B2M/FAS, TCR/B2M/CD38, TCR/B2M/CD38/NKG2A, TCR/B2M/NKG2A, TCR/B2M/FAS/CD38, or TCR/B2M/FAS/NKG2A knockout engineered cells.

In one example, the recombinant nucleic acid sequence of the chimeric receptor is integrated in the first exon of the constant region of the TCR α chain. For example, the recombinant nucleic acid sequence of the chimeric receptor is integrated in the first exon of the constant region of the TCR α chain, and the integration reduces endogenous TCR expression, and the expression of the integrated nucleic acid sequence in T cells is under the control of the endogenous TCR α chain promoter or the exogenous promoter.

The term “microwave ablation” (MWA): it is a local thermal ablation technology.

The term “gamma knife radiosurgery (GKRS): it is a kind of precise radiotherapy, which focuses gamma rays on the diseased tissue, making the diseased tissue necrosis or functional changes to achieve the purpose of treating disease.

The term “transcatheter arterial chemoembolization (TACE): the medicine is delivered into the arteries of the tumor through the catheter, to make the medicine work within the tumor, and at the same time, the blood supply of the tumor is cut off by embolizing the blood vessels of the tumor.

The term “second polypeptide” refers to the polypeptide expressed by immune cells described in the present application, including immune response inhibitors, cytokines, cytokine receptors, chemokines, chemokine receptors or a combination thereof. For example, the second polypeptide comprises a ligand binding domain of the receptors of interleukin-13 receptor alpha 2 (IL13Ra2), interleukin-2 receptor beta (IL2Rb), interleukin-18 receptor alpha (IL18Rα), and interleukin-18 receptor beta (IL18Rb), a transmembrane domain, and an intracellular signaling domain of interleukin-9 receptor alpha (IL9Ra).

The term “cytokine” refers to a general term for proteins released by cells that act as intercellular mediators on another cell, including: lymphokines, monokine, interleukin (IL), e.g., including: IL-1, IL-la, IL-2, IL-3, IL-4, IL-5, IL-6, IL-8, IL-9, IL-11, IL-12, IL15, IL-21, IL-18, IL-9, IL-23, or IL-36γ; Interferon IFNα2b; tumor necrosis factor (e.g., TNF-α or TNF-β), e.g., including: IL15, IL21, IL12, or IL7, or a combination thereof.

In one example, cytokine receptors comprise: IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, variants or a combination thereof.

In one example, the chemokine receptor molecule comprises a naturally occurring or recombinant chemokine receptor or a chemokine binding fragment thereof, examples include: CXC chemokine receptors (such as CXCR1, CXCR2, CXCR3, CXCR4, CXCR5, CXCR6, or CXCR7), CC chemokine receptors (such as CCR1, CCR2, CCR3, CCR4, CCR5, CCR6, CCR7, CCR8, CCR9, CCR10, or CCR11), CX3C chemokine receptors (such as CX3CR1), XC chemokine receptors (such as XCR1), or a chemokine binding fragment thereof, preferably CCR4, CXCR5, CXCL13, CCR2b, or CXCR2, or a combination thereof.

In one example, the chemokines comprise: CCL2, CCL5, CCL10, CCL14, CCL19, CCL20, CCL21, CXCL8, CXCL9, CXCL13, LEC, or a combination thereof, preferably CXCL9, CCL19, or CCL21, or a combination thereof.

In one example, the immune response inhibitor is an immune checkpoint inhibitor, and the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the inhibitor is preferably PD-1. In certain embodiments, the inhibitor is transforming growth factor 3 (TGF-0) receptor.

In one example, the immune response inhibitor is a dominant negative TGF-β receptor or “TGFβ receptor dominant negative form (DN form)”, which refers to a variant of TGF-β receptor that can compete with TGF-β receptor to bind TGF-β ligand (such as TGF-β 1), but cannot perform the signaling function of TGF-β receptor (such as TGF-βRII). For example, the intracellular signaling domain of the dominant negative TGF-β receptor is mutated, thereby losing the intracellular signaling ability. For example, the dominant negative TGF-β receptor lacks the intracellular signaling domain of TGF-β receptor. For example, TGF-β receptors are selected from TGF-βRII receptors. For example, the dominant negative TGF-β receptor comprises the amino acid sequence shown in SEQ ID NO: 64.

In one example, the DN form of the TGFβ receptor may comprise the extracellular domain of the TGFβ receptor or a ligand binding portion thereof, for example, amino acids 23 to 191 corresponding to the extracellular domain of the TGFβ receptor (GenBankNP_001020018.1). Cells expressing such a DN form of the TGFβ receptor lack the ability to signal transduction in cells or have reduced ability to signal transduction. For example, the DN form of the TGFβ receptor is a deletion mutant with deletion of intracellular domain, such as amino acids 213 to 592 of TGFβ receptor (GenBankNP_001020018.1) or part thereof, so that intracellular signal transduction mediated by TGFβ receptor is reduced or inhibited. For example, the DN form of the TGFβ receptor see e.g., Bottinger et al., EMBO J. 16:2621-2633 (1997), describing a DN form comprising TGFβ receptor extracellular and transmembrane domains; Foster et al., J. Immunother. 31:500-505 (2008); Bollard et al., Blood 99:3179-3187 (2002); Wieser et al., Mol. Cell. Biol. 13:7239-7247 (1993).

In one example, the immune response inhibitor is a chimeric TGF-β receptor, comprising: an extracellular domain of a TGFβ receptor and an intracellular domain of a non TGFβ receptor. The intracellular domain comprises the intracellular domain of immunostimulatory proteins or a functional fragments thereof, which are from TLR1, TLR2, TLR3, TLR4, TLR5, TLR6, TLR7, TLR8, TLR9, CD28, 41BB, 41BB, OX40, CD3ζ, CD40, CD27, or a combination thereof.

In one example, the extracellular domain of the chimeric TGF-β receptor comprises the extracellular domain of TGF-β receptor I and/or the extracellular domain of TGF-β receptor II; and the extracellular domain of TGF-β receptor II is preferred. The chimeric TGF-β receptor comprises an extracellular domain of the TGF-β receptor and an intracellular signaling domain of the receptor of IL-2 family proteins; among them, the intracellular signaling domain of the receptor of IL-2 family proteins is selected from the intracellular signaling domain of IL-2RG, IL-2RP, IL-7R, or IL-21R; the extracellular domain of TGF-β receptor is selected from the extracellular domain of TGF-β receptor I (TGF-β RI) and/or TGF-β receptor II (TGF-β RII); the chimeric TGF-β receptor comprises a first chimeric protein and a second chimeric protein, the first chimeric protein comprises the extracellular domain of TGF-β receptor I, the transmembrane domain of IL-2RG and the intracellular signaling domain of IL-2RG; the second chimeric protein comprises the extracellular domain of TGF-β receptor II, the transmembrane domain of IL-2RP and the intracellular signaling domain of IL-2RP, or the transmembrane domain of IL-7R and the intracellular signaling domain of IL-7R, or the transmembrane domain of IL-21R and the intracellular signaling domain of IL-21R.

In one example, the chimeric TGF-β receptor comprises a first chimeric protein and a second chimeric protein, and the first chimeric protein comprises the extracellular TGFβ1 binding domain of TGFβRII, the transmembrane domain of IL-12Rβ2, and the intracellular signaling domain IL-12Rβ2; the second chimeric protein comprises the extracellular TGFβ 1 binding domain of TGFβR1, the transmembrane domain of IL-12Rβ1, and the intracellular signaling domain of IL-12Rβ1.

In one example, the chimeric TGF-β receptor comprises a first chimeric protein and a second chimeric protein, and the first chimeric protein comprises the extracellular TGFβ1 binding domain of TGFβRII, the transmembrane domain of IL-7Ra, and the intracellular signaling domain of IL-7Ra; the second chimeric protein comprises the extracellular TGFβ1 binding domain of TGFβRI, the transmembrane domain of IL-2Rγ, and the intracellular signaling domain of IL-2Rγ.

In one example, the immune response inhibitor is a dominant negative form (DN form), comprising: (a) at least part of the extracellular domain of the immune checkpoint inhibitor, wherein the part comprises a ligand binding region, and (b) a transmembrane domain.

In one example, the inhibitor of immune response is PD-1 dominant negative form (DN form).

In one example, the second polypeptide comprises NKG2D, truncated NKG2D, the extracellular domain of truncated NKG2D, or variants thereof. For example, the second polypeptide comprises an antibody targeting NKG2D ligand or an antigen binding fragment thereof. For example, the second polypeptide comprises TIGIT, truncated TIGIT, the extracellular domain of truncated TIGIT or variants thereof, or SIRP-α or variants thereof, or antibodies targeting TIGIT ligands or antigen binding fragments thereof.

The term “TGF-β receptor” or “transforming growth factor 3 receptor”: there are three types of TGF-β receptors, i.e., TGF-β receptor I (TGF-βRI), TGF-β receptor II (TGF-βRII), and TGF-β receptor III (TGF-βRIII). TGF-βRI has the amino acid sequence corresponding to GenBank Gene ID: 7046, or a fragment thereof. TGF-β receptor II polypeptide can have the amino acid sequence corresponding to GenBank Gene ID: 7048, or a fragment thereof; or the amino acid sequence corresponding to GenBank No. NP_001020018.1 (GI:67782326), or a fragment thereof. TGF-β receptor III has the amino acid sequence corresponding to GenBank Gene ID: 7049, or a fragment thereof.

The term “GPC3”, phosphatidylinositol proteoglycan-3 (Glypican-3), is a cell surface protein that belongs to the heparan sulfate proteoglycan family. For example, human GPC3, NCBI GenBank Gene ID:2719. The GPC3 gene encodes a precursor core protein of about 70-kDa that can be cleaved by furin to produce a soluble amino terminal (N-terminus) peptide of about 40-kDa that can enter the blood and a carboxy terminal (C-terminus) peptide of about 30-kDa. GPC3 is expressed in liver cancer, melanoma, ovarian clear cell carcinoma, yolk sac tumor, neuroblastoma and other tumors. For example, anti-GPC3 chimeric antigen receptors comprise antigen binding units that bind to the C-terminus of GPC3. Subjects with tumors expressing GPC-3 may be referred to as patients with GPC3-positive tumors.

Cancers expressing GPC3 include but are not limited to liver cancer, gastric cancer, esophageal cancer, lung cancer, breast cancer, head and neck cancer, ovarian cancer, kidney cancer, bladder cancer, cervical cancer, pancreatic cancer, liposarcoma, testicular nonseminomatous germ cell cancer, melanoma, adenoma, adrenal cancer, schwannoma, malignant fibrous histiocytoma or any combinations thereof. GPC3-CAR-T can be used to target ovarian cancer, cholangiocarcinoma, mesothelioma, breast cancer, lung squamous cell carcinoma, cervical intraepithelial neoplasia, cervical squamous cell carcinoma, intrahepatic and extrahepatic cancers, gallbladder cancer, invasive ductal carcinoma, clear cell carcinoma, large eosinophil tumor, papillary carcinoma, adenocarcinoma, papillary carcinoma, as well as lobular and medullary carcinoma of the breast.

The method provided by the present application can be used to treat patients with GPC3-positive tumors. For example, GPC3-positive tumors comprise hepatocellular carcinoma, hepatic cell carcinoma, hepatoblastoma, rhabdomyosarcoma, malignant rhabdoid tumor, liposarcoma, Wilms tumor, York sac tumor, embryonal sarcoma of the liver, squamous cell carcinoma of the lung, merkel cell carcinoma, lung cancer, breast cancer, colo-rectal cancer, or brain tumor.

In one example, the extracellular antigen binding region of GPC3-CAR can target the N-terminus, C-terminus, or any part from the N-terminus to the C-terminus of GPC3. The C-terminus of GPC3 can be covalently linked to the cell membrane via a glycosylphosphatidylinositol (GPI) anchor. For example, the C-terminus may comprise from about 1 to about 800 bases. The C-terminus may comprise about 1, 50, 100, 150, 300, 500, 600, up to about 800 bases from the C-terminus. For example, the extracellular antigen binding region can target the GPI anchor of GPC3.

In one example, CAR comprises an antibody that recognize human tumor antigens, a transmembrane domain, and an intracellular domain. For example, CAR comprises a variable region or scFv that recognizes human tumor antigens, the transmembrane domain of CD8 or CD28, the intracellular signaling domain of CD28 or CD137, and the intracellular signaling domain of CD3ζ.

For example, CAR comprises the variable region or scFv that recognizes human tumor antigens, the transmembrane domain of CD8 or CD28, the intracellular signaling domain of CD28, the intracellular signaling domain of CD137, and the intracellular signaling domain of CD3ζ. For example, CAR comprises the variable region or scFv, the hinge region of CD8 or CD28 or IgGI or IgG4, the transmembrane domain of CD8 or CD28, the intracellular signaling domain of CD28 or CD137, and the intracellular signaling domain of CD3ζ, which are connected sequentially. For example, CAR comprises the variable region or scFv, the hinge region of CD8 or CD28 or IgGI or IgG4, the transmembrane domain of CD8 or CD28, the intracellular signaling domain of CD28, the intracellular signaling domain of CD137, and the intracellular signaling domain of CD3ζ, which are connected sequentially. For example, CAR comprises the variable region or scFv, the hinge region of CD28, the transmembrane domain of CD28, the intracellular signaling domain of CD28, and the intracellular signaling domain of CD3ζ, which are connected sequentially. For example, CAR comprises the variable region or scFv, the hinge region of CD8, the transmembrane domain of CD8, the intracellular signaling domain of CD137, and the intracellular signaling domain of CD3ζ, which are connected sequentially. For example, CAR comprises the variable region or scFv, the hinge region of CD8, the transmembrane domain of CD28, the intracellular signaling domain of CD28, and the intracellular signaling domain of CD3ζ, which are connected sequentially. For example, CAR comprises the variable region or scFv, the hinge region of IgG4, the transmembrane domain of CD28, the intracellular signaling domain of CD28, and the intracellular signaling domain of CD3ζ, which are connected sequentially. For example, CAR comprises the variable region or scFv, the hinge region of CD8, the transmembrane domain of CD28, the intracellular signaling domain of CD28, the intracellular signaling domain of CD137, and the intracellular signaling domain of CD3ζ, which are connected sequentially. For example, CAR comprises the variable region or scFv, the hinge region of IgGI, the transmembrane domain of CD28, the intracellular signaling domain of CD137, and the intracellular signaling domain of CD3ζ, which are connected sequentially. For example, CAR comprises the variable region or scFv, the hinge region of IgG4, the transmembrane domain of CD28, the intracellular signaling domain of CD137, and the intracellular signaling domain of CD3ζ, which are connected sequentially.

In one example, the GPC3-CAR comprises a variable region (e.g., SEQ ID NO: 37, 38, 23, 24, 26, or 27) or scFv (e.g., SEQ ID NO: 1, 2, 3, 4, 5, 6, 7, 8, or 62) that recognizes the C-terminus of the human GPC3 protein. For example, GPC3-CAR comprises the variable region or scFv that recognizes the C-terminus of human GPC3 protein, the hinge region of CD8 or CD28 or IgGI or IgG4, the transmembrane domain of CD8 or CD28, the intracellular signaling domain of CD28 or CD137, and the intracellular signaling domain of CD3ζ, which are connected sequentially. For example, CAR comprises the variable region or scFv, the hinge region of CD8 or CD28 or IgGI or IgG4, the transmembrane domain of CD8 or CD28, the intracellular signaling domain of CD28, the intracellular signaling domain of CD137, and the intracellular signaling domain CD3ζ, which are connected sequentially. For example, GPC3-CAR comprises the variable region or scFv that recognizes the C-terminus of human GPC3 protein, the hinge region of CD28, the transmembrane domain of CD28, the intracellular signaling domain of CD28, and the intracellular signaling domain of CD3ζ, which are connected sequentially. For example, GPC3-CAR comprises the variable region or scFv that recognizes the C-terminus of human GPC3 protein, the hinge region of CD8, the transmembrane domain of CD8, the intracellular signaling domain of CD137, and the intracellular signaling domain of CD3ζ, which are connected sequentially. For example, CAR comprises the variable region or scFv, the hinge region of CD8, the transmembrane domain of CD28, the intracellular signaling domain of CD28, and the intracellular signaling domain of CD3ζ, which are connected sequentially. For example, CAR comprises the variable region or scFv, the hinge region of CD8, the transmembrane domain of CD28, the intracellular signaling domain of CD28, the intracellular signaling domain of CD137, and the intracellular signaling domain of CD3ζ, which are connected sequentially. For example, CAR comprises the variable region or scFv, the hinge region of IgGI, the transmembrane domain of CD28, the intracellular signaling domain of CD137, and the intracellular signaling domain of CD3ζ, which are connected sequentially.

For example, the hinge region of CD8 comprises an amino acid sequence as shown in SEQ ID NO:9 or having at least 90% identity with SEQ ID NO:9. The hinge region of IgGI comprises an amino acid sequence as shown in SEQ ID NO:55 or having at least 90% identity with SEQ ID NO:55. The hinge region of IgG4 comprises an amino acid sequence as shown in SEQ ID NO:56 or 57 or having at least 90% identity with SEQ ID NO:56 or 57. The transmembrane domain of CD8 comprises an amino acid sequence as shown in SEQ ID NO: 15 or having at least 90% identity with SEQ ID NO:15. The transmembrane domain of CD28 comprises an amino acid sequence as shown in SEQ ID NO:10 or 58 or having at least 90% identity with SEQ ID NO:10 or 58. The intracellular domain of CD28 comprises an amino acid sequence as shown in SEQ ID NO:11 or having at least 90% identity with SEQ ID NO:11. The intracellular domain of CD137 comprises an amino acid sequence as shown in SEQ ID NO:13 or having at least 90% identity with SEQ ID NO:13. The intracellular signaling domain of CD3ζ comprises an amino acid sequence as shown in SEQ ID NO:12 or 59 or having at least 90% identity with SEQ ID NO:12 or 59.

In one example, the antigen binding unit of the GPC3-CAR comprises a sequence having at least 90% sequence identity with SEQ ID NO: 1, 2, 3, 4, 5, 6, 7, 8 or 62. In one example, HCDR1, HCDR2, HCDR3, LCDR1, LCDR2 and LCDR3 of the antigen binding unit of the GPC3-CAR successively comprise: (1) SEQ ID NO:31, 32, 33, 34, 35 and 36; or (2) SEQ ID NO:17, 18, 19, 20, 21, 22; or (3) SEQ ID NO:17, 18, 19, 25, 21, 22.

In one example, the VH and VL of the antigen binding unit of the GPC3-CAR comprise: the sequences having at least 90% sequence identity with SEQ ID NO:37 and 38; or SEQ ID NO:23 and 24; or SEQ ID NO:26 and 27. In one example, GPC3-CAR comprises any of the following sequences:
(1) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:15, the costimulatory signaling domain shown in SEQ ID NO: 11, and the primary signaling domain shown in SEQ ID NO:12; or
(2) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:15, the costimulatory signaling domain shown in SEQ ID NO: 13, and the primary signaling domain shown in SEQ ID NO:12; or
(3) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:10, the costimulatory signaling domain shown in SEQ ID NO: 11, and the primary signaling domain shown in SEQ ID NO:12; or
(4) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:10, the costimulatory signaling domain shown in SEQ ID NO: 13, and the primary signaling domain shown in SEQ ID NO:12; or
(5) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:15, the costimulatory signaling domain shown in SEQ ID NO:11, the costimulatory signaling domain shown in SEQ ID NO:13, and the primary signaling domain shown in SEQ ID NO: 12; or
(6) comprising the extracellular domain shown in SEQ ID NO: 1, 2, 3, 4, 5, 6, 7, 8 or 62, the hinge domain shown in SEQ ID NO:9, the transmembrane domain shown in SEQ ID NO:10, the costimulatory signaling domain shown in SEQ ID NO:11, the costimulatory signaling domain shown in SEQ ID NO:13, and the primary signaling domain shown in SEQ ID NO: 12; or
(7) comprising the extracellular domain shown in SEQ ID NO: 1, 2, 3, 4, 5, 6, 7, 8 or 62, the hinge domain shown in SEQ ID NO:55, the transmembrane domain shown in SEQ ID NO:10, the costimulatory signaling domain shown in SEQ ID NO: 13, and the primary signaling domain shown in SEQ ID NO:12; or
(8) comprising the extracellular domain shown in SEQ ID NO: 1, 2, 3, 4, 5, 6, 7, 8 or 62, the hinge domain shown in SEQ ID NO:56 or 57, the transmembrane domain shown in SEQ ID NO:10, the costimulatory signaling domain shown in SEQ ID NO:13, and the primary signaling domain shown in SEQ ID NO: 12; or
(9) comprising the extracellular domain shown in SEQ ID NO: 1, 2, 3, 4, 5, 6, 7, 8 or 62, the hinge domain shown in SEQ ID NO:56 or 57, the transmembrane domain shown in SEQ ID NO:10, the costimulatory signaling domain shown in SEQ ID NO:11, and the primary signaling domain shown in SEQ ID NO: 12.

In one example, GPC3-CAR comprises sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 28, 29, 30, 60, or 61.

The term “CLDN18.2” is a type II isoform of claudin18, which is a member of the claudin family of cell surface proteins. Claudin is an important component of tight cell junctions, which forms a paracellular barrier that controls the flow of molecules between cells. Different claudins are expressed on different tissues, and their altered functions have been associated with the formation of cancer in these tissues. In normal tissues, the expression of CLDN18.2 is restricted to the epithelial cells of the gastric mucosa. CLDN18.2 is expressed in gastric cancer and its metastases, pancreatic, esophageal, ovarian and lung tumors.

The human CLDN18.2 protein has 261 amino acids (NCBI, NP_001002026.1). CLDN18.2 exists as a tetraspanin protein with N-terminus and C-terminus in the cytoplasm. CLDN18.2 has two extracellular loops.

A CLDN18.2 antigen binding unit specifically binds to claudin 18.2, fragments or variants thereof. For example, the CLDN18.2 antigen binding unit specifically binds to human CLDN18.2. For example, the CLDN18.2 antigen binding unit specifically binds to the extracellular domain of CLDN18.2. For example, the CLDN18.2 antigen binding unit specifically binds to the first extracellular loop of CLDN18.2. For example, the CLDN18.2 antigen binding unit specifically binds to the second extracellular loop of CLDN18.2. For example, the CLDN18.2 antigen binding unit specifically binds to the first and second extracellular loops of CLDN18.2. For example, the affinity of the CLDN18.2 antigen binding unit binding to CLDN18.2 is at least 20-fold greater than that of the antibody binding to CLDN18.1. For example, the affinity of the CLDN18.2 antigen binding unit binding to CLDN18.2 is at least 50-fold greater than that of the antibody binding to CLDN18.1. For example, the affinity of the CLDN18.2 antigen binding unit binding to CLDN18.2 is at least 100-fold greater than that of the antibody binding to CLDN18.1. For example, no binding of CLDN18.2 antigen binding unit to CLDN18.1 is detected.

In one example, CLDN18.2-CAR comprises a variable region (e.g., SEQ ID NO: 45, 46) or scFv (e.g., SEQ ID NO: 47, 48, 49, or 50) or VHH (e.g., SEQ ID NO:63) that recognizes human CLDN18.2 protein. For example, CLDN18.2-CAR comprises: the variable region or scFv, the hinge region of CD8 or CD28 or IgGI or IgG4, the transmembrane domain of CD8 or CD28, the intracellular signaling domain of CD28 or CD137, and the intracellular signaling domain of CD3ζ, which are connected sequentially. For example, CLDN18.2-CAR comprises: the variable region or scFv, the hinge region of CD8 or CD28 or IgGI or IgG4, the transmembrane domain of CD8 or CD28, the intracellular signaling domain of CD28, the intracellular signaling domain of CD137, and the intracellular signaling domain of CD3ζ, which are connected sequentially. For example, CLDN18.2-CAR comprises: the variable region or scFv, the hinge region of CD28, the transmembrane domain of CD28, the intracellular signaling domain of CD28, and the intracellular signaling domain of CD3ζ, which are connected sequentially. For example, CLDN18.2-CAR comprises: the variable region or scFv, the hinge region of CD8, the transmembrane domain of CD8, the intracellular signaling domain of CD137, and the intracellular signaling domain of CD3ζ, which are connected sequentially. For example, CLDN18.2-CAR comprises: the variable region or scFv, the hinge region of CD8, the transmembrane domain of CD28, the intracellular signaling domain of CD28, and the intracellular signaling domain of CD3ζ, which are connected sequentially. For example, CLDN18.2-CAR comprises: the variable region or scFv, the hinge region of CD8, the transmembrane domain of CD28, the intracellular signaling domain of CD28, the intracellular signaling domain of CD137, and the intracellular signaling domain of CD3ζ, which are connected sequentially. For example, CLDN18.2-CAR comprises: the variable region or scFv, the hinge region of IgGI, the transmembrane domain of CD28, the intracellular signaling domain of CD137, and the intracellular signaling domain of CD3ζ, which are connected sequentially.

In one example, the antigen binding unit of the CLDN18.2-CAR comprises a sequence having at least 90% sequence identity with SEQ ID NO: 47, 48, 49, 50 or 63.

In one example, the antigen binding unit of the CLDN18.2-CAR comprises HCDR1, HCDR2, HCDR3, LCDR1, LCDR2, and LCDR3, which successively comprise sequences shown in SEQ ID NO:39, 40, 41, 42, 43, and 44.

In one example, the antigen binding unit of the CLDN18.2-CAR comprises a sequence having at least 90% sequence identity with the VH shown in SEQ ID NO:45 and the VL shown in SEQ ID NO:46.

In one example, the antigen binding unit of the CLDN18.2-CAR comprises those antibody sequences disclosed by WO2020135674A1, WO2021129765A1 and WO2022135578A1.

In one example, CLDN18.2-CAR comprises any of the following sequences:
(1) having the extracellular domain shown in SEQ ID NO: 47, 48, 49, 50 or 63, the hinge domain shown in SEQ ID NO:9, the transmembrane domain shown in SEQ ID NO:15, the costimulatory signaling domain shown in SEQ ID NO: 11, and the primary signaling domain shown in SEQ ID NO:12; or
(2) having the extracellular domain shown in SEQ ID NO: 47, 48, 49, 50 or 63, the hinge domain shown in SEQ ID NO:9, the transmembrane domain shown in SEQ ID NO:15, the costimulatory signaling domain shown in SEQ ID NO: 13, and the primary signaling domain shown in SEQ ID NO:12; or
(3) having the extracellular domain shown in SEQ ID NO: 47, 48, 49, 50 or 63, the hinge domain shown in SEQ ID NO:9, the transmembrane domain shown in SEQ ID NO:10, the costimulatory signaling domain shown in SEQ ID NO: 11, and the primary signaling domain shown in SEQ ID NO:12; or
(4) having the extracellular domain shown in SEQ ID NO: 47, 48, 49, 50 or 63, the hinge domain shown in SEQ ID NO:9, the transmembrane domain shown in SEQ ID NO:10, the costimulatory signaling domain shown in SEQ ID NO: 13, and the primary signaling domain shown in SEQ ID NO:12; or
(5) having the extracellular domain shown in SEQ ID NO: 47, 48, 49, 50 or 63, the hinge domain shown in SEQ ID NO:9, the transmembrane domain shown in SEQ ID NO:15, the costimulatory signaling domain shown in SEQ ID NO:11, the costimulatory signaling domain shown in SEQ ID NO:13, and the primary signaling domain shown in SEQ ID NO: 12; or
(6) having the extracellular domain shown in SEQ ID NO: 47, 48, 49, 50 or 63, the hinge domain shown in SEQ ID NO:9, the transmembrane domain shown in SEQ ID NO:10, the costimulatory signaling domain shown in SEQ ID NO:11, the costimulatory signaling domain shown in SEQ ID NO:13, and the primary signaling domain shown in SEQ ID NO: 12, or
(7) comprising the extracellular domain shown in SEQ ID NO: 47, 48, 49, 50 or 63, the hinge domain shown in SEQ ID NO:55, the transmembrane domain shown in SEQ ID NO:10, the costimulatory signaling domain shown in SEQ ID NO: 13, and the primary signaling domain shown in SEQ ID NO:12; or
(8) comprising the extracellular domain shown in SEQ ID NO: 47, 48, 49, 50 or 63, the hinge domain shown in SEQ ID NO:56 or 57, the transmembrane domain shown in SEQ ID NO:10, the costimulatory signaling domain shown in SEQ ID NO: 13, and the primary signaling domain shown in SEQ ID NO:12; or
(9) comprising the extracellular domain shown in SEQ ID NO: 47, 48, 49, 50 or 63, the hinge domain shown in SEQ ID NO:56 or 57, the transmembrane domain shown in SEQ ID NO:10, the costimulatory signaling domain shown in SEQ ID NO: 11, and the primary signaling domain shown in SEQ ID NO:12.

In one example, the anti-CLDN18.2-CAR comprises a sequence having 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 51, 52, 53, or 54.

The present application provides a method for treating a subject with CLDN18.2-positive pancreatic cancer. For example, after the local treatment or the local treatment combined with the other adjuvant treatment, CLDN18.2-CAR-T cells are administered by intravenous infusion. For example, after the local treatment or the local treatment combined with the other adjuvant treatment, if there is no imaging recurrence and/or metastasis, CLDN18.2-CAR-T cells are administered by intravenous infusion. For example, after the local treatment or the local treatment combined with the other adjuvant treatment, if there is no imaging recurrence and/or metastasis, and no enlargement of the other minimal tumor lesions without the local treatment is detected by imaging, the CLDN18.2-CAR-T cells are administered by intravenous infusion. For example, after the local treatment or the local treatment combined with the other adjuvant treatment, if the CA19-9 level in peripheral blood is higher than the upper limit of the normal range, the CLDN18.2-CAR-T cells are administered by intravenous infusion. For example, after the local treatment or the local treatment combined with the other adjuvant treatment, if the level of CA19-9 in peripheral blood increases progressively, the CLDN18.2-CAR-T cells are administered by intravenous infusion. For example, after the local treatment or the local treatment combined with the other adjuvant treatment, if the level of CA19-9 in peripheral blood is higher than the upper limit of the normal range and increases progressively, the CLDN18.2-T cells are administered by intravenous infusion. For example, after the local treatment or the local treatment combined with the other adjuvant treatment, if the level of CA19-9 in peripheral blood increases progressively and there is no imaging recurrence and/or metastasis, the CLDN18.2-CAR-T cells are administered by intravenous infusion. For example, after the local treatment or the local treatment combined with the other adjuvant treatment, if the level of CA19-9 in peripheral blood increases progressively, there is no imaging recurrence and/or metastasis, and no enlargement of the other minimal tumor lesions without the local treatment is detected by imaging, the CLDN18.2-CAR-T cells are administered by intravenous infusion. For example, after the local treatment or the local treatment combined with the other adjuvant treatment, if the CA19-9 level in peripheral blood is higher than the upper limit of the normal range and increases progressively, and there is no imaging recurrence and/or metastasis, the CLDN18.2-CAR-T cells are administered by intravenous infusion. For example, after the local treatment or the local treatment combined with the other adjuvant treatment, if the CA19-9 level in peripheral blood is higher than the upper limit of the normal range and increases progressively, there is no imaging recurrence and/or metastasis, and no enlargement of the other minimal tumor lesions without the local treatment is detected by imaging, the CLDN18.2-CAR-T cells are administered by intravenous infusion. For example, after the local treatment or the local treatment combined with the other adjuvant treatment, if ctDNA or CTC is detected in peripheral blood and there is no imaging recurrence and/or metastasis, the CLDN18.2-CAR-T cells are administered by intravenous infusion. For example, after the local treatment or the local treatment combined with the other adjuvant treatment, if ctDNA or CTC is detected in peripheral blood, there is no imaging recurrence and/or metastasis, and no enlargement of the other minimal tumor lesions without the local treatment is detected by imaging, the CLDN18.2-CAR-T cells are administered by intravenous infusion. For example, the local treatment comprises: surgical treatment comprising a radical surgery for pancreatic cancer (e.g., pancreatoduodenectomy, tail pancreatectomy, pancreatectomy, etc.), or an interventional treatment (e.g., arterial infusion chemotherapy, particle implantation, radiofrequency ablation, etc.).

The present application provides a method for treating a subject with CLDN18.2-positive pancreatic cancer. For example, after radical surgery for pancreatic cancer or radical surgery combined with the other adjuvant treatment, if the level of CA19-9 in peripheral blood increases progressively and there is no imaging recurrence and/or metastasis, the CLDN18.2-CAR-T cells are administered by intravenous infusion. For example, after the radical surgery for pancreatic cancer or the radical surgery combined with the other adjuvant treatment, if ctDNA or CTC is detected in peripheral blood and there is no imaging recurrence and/or metastasis, the CLDN18.2-CAR-T cells are administered by intravenous infusion. For example, the radical surgery for pancreatic cancer comprises pancreatoduodenectomy, tail pancreatectomy, or pancreatectomy.

The present application provides a method for treating a subject with CLDN18.2-positive gastric cancer or gastroesophageal junction tumor. For example, after the local treatment or the local treatment combined with the other adjuvant treatment, the CLDN18.2-CAR-T cells are administered by intravenous infusion. For example, after the local treatment or the local treatment combined with the other adjuvant treatment, if there is no imaging recurrence and/or metastasis, the CLDN18.2-CAR-T cells are administered by intravenous infusion. For example, after the local treatment or the local treatment combined with the other adjuvant treatment, if there is no imaging recurrence and/or metastasis, and no enlargement of the other minimal tumor lesions without the local treatment is detected by imaging, the CLDN18.2-CAR-T cells are administered by intravenous infusion. For example, after the local treatment or the local treatment combined with the other adjuvant treatment, if ctDNA or CTC is detected in peripheral blood and there is no imaging recurrence and/or metastasis, the CLDN18.2-CAR-T cells are administered by intravenous infusion. For example, after the local treatment or the local treatment combined with the other adjuvant treatment, if ctDNA or CTC is detected in peripheral blood, there is no imaging recurrence and/or metastasis, and no enlargement of the other minimal tumor lesions without the local treatment is detected by imaging, the CLDN18.2-CAR-T cells are administered by intravenous infusion. For example, the local treatment comprises: surgical treatment comprising a radical surgery for gastric cancer or gastroesophageal junction tumor (for example, radical surgery for gastric cancer, radical surgery for gastroesophageal cancer, etc.), or an interventional treatment (for example, arterial infusion chemotherapy, particle implantation, radiofrequency ablation, etc.).

The present application provides a method for treating a subject with CLDN18.2-positive gastric cancer or gastroesophageal junction tumor. For example, after radical surgery for gastric cancer or gastroesophageal cancer, or radical surgery combined with the other adjuvant treatment, if there is no imaging recurrence and/or metastasis, the CLDN18.2-CAR-T cells are administered by intravenous infusion. For example, after the radical surgery for gastric cancer or gastroesophageal cancer, or the radical surgery combined with the other adjuvant treatment, if ctDNA or CTC is detected in peripheral blood and there is no imaging recurrence and/or metastasis, the CLDN18.2-CAR-T cells are administered by intravenous infusion.

The method provided by the present application can be used to treat patients with CLDN18.2-positive tumors. For example, CLDN18.2-positive tumors comprise gastrointestinal tumor, gastric adenocarcinoma, gastroesophageal junction adenocarcinoma, gastric cancer, pancreatic cancer, gastroesophageal junction cancer, esophageal cancer, pancreatic ductal adenocarcinoma, or ovarian carcinoma. For example, CLDN18.2-positive tumors comprise digestive system tumors.

The level of CA19-9 after pancreatic cancer surgery is correlated with the prognosis of patients. The abnormality of CA19-9 after surgery often indicates the existence of occult distant metastasis in patients. The increase of CA19-9 after surgery is a strong predictor of early recurrence of pancreatic cancer. Even if there is no recurrence under the imaging, treatment decisions and salvage treatment can be considered according to CA19-9.

AFP is a kind of carcinoembryonic glycoprotein, which is highly related to the occurrence and progression of HCC. About 70% of HCC patients have elevated serum AFP levels, and high AFP levels are related to the more aggressive HCC tissue phenotype (for example, vascular invasion, poor differentiation, satellite foci, etc.), which is one of the risk factors of HCC. AFP has been applied in the screening, diagnosis, efficacy monitoring and prognosis prediction of HCC. Serum AFP level after local treatment is closely related to imaging recurrence. Patients with a rising trend of AFP have a high probability of subsequent development of imaging recurrence. Patients with persistently elevated AFP have a shorter time to imaging recurrence.

The term “tumor antigen” refers to an antigen that emerging or overexpressed during the occurrence or development of hyperproliferative diseases. For example, the hyperproliferative disorder in the present application refers to cancer. The hyperproliferative disease is called cancer or tumor. Tumor antigens include but are not limited to: thyroid stimulating hormone receptor (TSHR); CD171; CS-1; C-type lectin like molecule-1; Ganglioside GD3; Tn antigen; CD19; CD20; CD22; CD30; CD70; CD123; CD138; CD33; CD44; CD44v7/8; CD38; CD44v6; B7H3 (CD276), B7H6; KIT (CD117); interleukin-13 receptor subunit alpha (IL-13Ru); interleukin-11 receptor alpha (IL-11Ru); prostate stem cell antigen (PSCA); prostate specific membrane antigen (PSMA); carcinoembryonic antigen (CEA); NY-ESO-1; HIV-1 Gag; MART-1; gp100; tyrosinase; mesothelin; EpCAM; protease serine 21 (PRSS21); vascular endothelial growth factor receptor, vascular endothelial growth factor receptor 2 (VEGFR2); Lewis (Y) antigen; CD24; platelet derived growth factor receptor beta (PDGFR-β); stage specific embryonic antigen-4 (SSEA-4); cell surface associated mucin 1 (MUC1), MUC6; epidermal growth factor receptor family and mutants thereof (EGFR, egfr2, ERBB3, ErbB4, EGFRvIII); neural cell adhesion molecule (NCAM); carbonic anhydrase IX (CAIX); LMP2; ephrin type A receptor 2 (EphA2); fucosyl GM1; sialyl Lewis adhesion molecule (sLE); ganglioside GM3 (aNeu5Ac (2-3) bDGalp(1-4)bDGlcp(1-1)Cer; TGS5; high molecular weight melanoma associated antigen (HMWMAA); o-acetyl GD2 Ganglioside (OAcGD2); folate receptor; tumor vascular endothelial marker 1 (TEM1/CD248); tumor vascular endothelial marker 7-related (TEM7R); Claudin 6, Claudin18.2, Claudin18.1; ASGPR1; CDH16; 5T4; 8H9; αvβ6 integrin; B cell maturation antigen (BCMA); CA9; kappa light chain; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variants in the tumor necrosis zone; G protein coupled receptor class C group 5 member D (GPRC5D); X chromosome open reading frame 61 (CXORF61); CD97; CD179a; anaplastic lymphoma kinase (ALK); polysialic acid; placenta specific 1 (PLAC1); the hexose moiety of globoH glycoceramide (globoH); mammary differentiation antigen (NY-BR-1); uroplakin 2 (UPK2); hepatitis A virus cellular receptor 1 (HAVCR1); adrenoceptor 03 (ADRB3); pannexin 3 (PANX3); G protein coupled receptor 20 (GPR20); lymphocyte antigen 6 complex locus K9 (ly6k); olfactory receptor 51E2 (OR51E2); TCRγ alternative reading frame protein (TARP); Wilms tumor protein (WT1); ETS translocation variant gene 6 (ETV6-AML); sperm protein 17 (SPA17); X antigen family member 1A (XAGE1); angiopoietin binds cell surface receptor 2 (Tie2); melanoma cancer testis antigen-1 (MAD-CT-1); melanoma cancer testis antigen-2 (MAD-CT-2); Fos associated antigen 1; p53 mutants; human telomerase reverse transcriptase (hTERT); sarcoma translocation breakpoints; melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (Pax3); androgen receptor; cyclin B1; V-myc avian myelocytomatosis viral oncogene neuroblastoma derived homolog (MYCN); Ras homolog family member C (RhoC); cytochrome P450 1B1 (CYP1B1); CCCTC binding factor (zinc finger protein)-like (BORIS); squamous cell carcinoma antigen 3 (SART3) recognized by T cells; paired box protein Pax-5 (PAX5); proacrosin binding protein sp32 (OYTES1); lymphocyte specific protein tyrosine kinase (LCK); A kinase anchored protein 4 (AKAP-4); synovial sarcoma x breakpoint 2 (SSX2); CD79a; CD79b; CD72; leukocyte associated immunoglobulin like receptor 1 (LAIR1); Fc fragment of IgA receptor (FCAR); leukocyte immunoglobulin like receptor subfamily member 2 (LILRA2); CD300 molecule like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); phosphatidylinositol proteoglycan-3 (GPC3); Fc receptor like 5 (FCRL5); immunoglobulin lambda like polypeptide 1 (IGLL1).

In one example, tumor antigen refers to the antigen expressed on the cell membrane of tumor cells.

In one example, tumor antigens comprise B7H3, GPC3, Claudin 6, Claudin18.2, FAP, mesothelin, NKG2D ligand, NKG2A, CD94, FCRH5, IL13Ra2, GD2, EpCAM, CEA, MUC1, MSLN, PSCA, AFP, ERBB2, EGFR or mutants thereof.

The term “antigen binding domain or antigen binding unit” refers to molecules that specifically bind to antigenic determinants, comprising immunoglobulin molecules and the immunoactive part of immune molecules, that is, molecules comprising antigen binding sites that specifically bind to antigens (“immune responses”). If the antigen binding domain binds to the antigen with a greater affinity (or avidity) than it binds to other reference antigens (comprising polypeptides or other substances), the antigen binding domain “specifically binds” to the antigen or is “immunoreactive” to the antigen. It also comprises any molecular structure comprising a polypeptide chain with a specific shape that fits and recognizes the epitope, in which one or more non covalent binding interactions stabilize the complex between the molecular structure and the epitope. For example, it comprises antibodies. For example, it comprises D-domain polypeptide (DDPP), see CN111727250A.

The term “antibody” is used in the broadest sense herein and comprises various antibody structures, including but not limited to monoclonal antibodies, polyclonal antibodies, multispecific antibodies (e.g., bispecific antibodies), domain antibodies, and antibody fragments thereof that can specifically bind to antigens or antigenic determinant, as long as they display the desired antigen binding activity. The term “antibody fragment” refers to a molecule different from the intact antibody, which comprises the portion of the intact antibody that binds to the antigen to which the intact antibody binds. Examples of antibody fragments include, but are not limited to, (i) Fab fragments consisting of VL, VH, CL, and CH1 domains, including Fab′ and Fab′-sh, (ii) FD fragments consisting of VH and CH1 domains, (iii) Fv fragments consisting of VL and VH domains of a single antibody; (iv) dAb fragments consisting of a single variable region; (v) F(ab′)2 fragment, a bivalent fragment comprising 2 linked Fab fragments; (vi) antigen binding sites of single chain Fv molecules; (vii) bispecific single chain Fv dimers; (viii) “bispecific antibodies” or “trispecific antibodies”, multivalent or multispecific fragments constructed by gene fusion; and (ix) scFvs genetically fused with the same or different antibodies. For example, antibodies are selected from: full antibody, scFv, single domain antibody, Fab fragment, Fab′ fragment, Fv fragment, F(ab′)2 fragment, Fd fragment, sdAb, multifunctional antibody, scFv-FC antibody or IgG4 antibody.

The terms “recognize”, “bind” and “target” are used interchangeably to refer to selective binding to target antigens. For example, binding to target cells refers to binding to target antigens (e.g., target molecules) on target cells.

The term “scFv” refers to a fusion protein comprising at least one antibody fragment of a light chain variable region and at least one antibody fragment of a heavy chain variable region, wherein the light chain and the heavy chain variable regions are adjacent (e.g., linked by a synthetic linker, such as a short flexible polypeptide linker), and can be expressed as a single chain polypeptide, and wherein the scFv retains the specificity of the intact antibody comprising the scFv. Unless specified, as used herein, scFv can have the VL and VH variable regions in any order (e.g., relative to the N-terminus and C-terminus of the polypeptide), and scFv may comprise VL-linker-VH or may comprise VH-linker-VL. The antigen binding function of antibodies may be performed by fragments of naturally occurring antibodies.

The term “variable region or variable domain” refers to the domain of the heavy or light chain of the antibody involved in antibody antigen binding.

The term “hypervariable region” or “complementarity determining region” or “CDR” refers to each region of the variable domain of an antibody in which the sequence is hypervariable, and/or forms a structurally determined loop (“hypervariable loop”), and/or comprises residues that contact with the antigen (“antigen contacts”). Typically, antibodies comprise six CDRs: three of the VH (HCDR1, HCDR2, and HCDR3) and three of the VL (LCDR1, LCDR2, and LCDR3).

The term “single domain antibody (sdAb)” also known as “VHH”, or “VHH polypeptide”, comprises a variable VHH domain responsible for antigen recognition.

The term “chimeric receptor” refers to a fusion molecule formed by linking DNA fragments from different sources or the corresponding cDNAs of proteins by gene recombination technology, comprising a extracellular domain, a transmembrane domain and a intracellular domain. Chimeric receptors include but are not limited to chimeric antigen receptors (CARs) and recombinant TCR receptors. For example, chimeric receptors comprise at least two or three intracellular signaling domains. The intracellular signaling domain is selected from the intracellular signaling domain of CD3, CD28, 4-1BB, OX40, DAP10 or ICOS.

The term “chimeric antigen receptor” (CAR) comprises at least one or two or more extracellular antigen binding domains, transmembrane domains, and intracellular domains. For example, CAR comprises antigen binding domains, transmembrane domains, and intracellular domains. For example, transmembrane domains comprise: transmembrane domain of CD8 or transmembrane domain of CD28. For example, the transmembrane domain of CD8 comprises a transmembrane domain of CD8a or a transmembrane domain of CD80. Intracellular domains comprises functional signaling domains of stimulatory and/or costimulatory molecules. For example, stimulatory molecules is (chain (e.g., CD3Z) that bind to the T cell receptor complex. For example, the intracellular domain further comprises functional signaling domains of one or more costimulatory molecules, such as 4-1BB (i.e., CD137), CD27, and/or CD28. For example, polypeptide groups are linked to each other. For example, the intracellular signaling domain (or domain) can be selected from the intracellular costimulatory domain of any one or more of the following polypeptides: CD27, CD28, TNFRSF9, TNFRSF4, TNFRSF8, TNFRSF14, TNFRSF18, CD40LG, ICOS, ITGB2, CD2, CD7, KLRC2, HAVCR1, LGALS9, CD83. For example, the CAR further comprises a hinge region. For example, the hinge region comprises: hinge region of CD8, hinge region of CD28, hinge region of IgGI or hinge region of IgG4. For example, the hinge region of CD8 comprises a CD8a hinge region or a CD80 hinge region.

The term “recombinant T cell receptor (recombinant TCR)” refers to a chimeric receptor comprising one or more TCR subunits. For example, the recombinant TCR comprises at least a portion of the extracellular domain of TCR subunit, transmembrane domain and intracellular domain of TCR subunit, wherein the TCR subunits are effectively linked to the antigen binding domain. For example, TCR subunits in recombinant TCRs comprise CD3ζ, CD3ε, CD3γ, CD3δ, TCRα, TCRβ, TCRγ, and/or TCRδ subunits. For example, a recombinant TCR may be integrated into TCR/CD3 complexes expressed on T cells. For example, the recombinant TCR comprises constant regions and intracellular domains of TCR α and TCR β subunits, and the constant regions of the subunit are effectively linked to the antigen binding domains. For example, the recombinant TCR comprises the constant regions and intracellular domains of TCRγ and TCRδ subunits, and the constant regions of the subunits are effectively linked to the antigen binding domains. For example, the recombinant TCR comprises CD3ζ, CD3ε, CD3γ, or CD3δ subunits, and the extracellular domains of the subunits are effectively linked to the antigen binding domains.

The term “primary signaling domain” or “first grade signaling domain” regulates the initial activation of the TCR complex in a stimulatory manner. On the one hand, the primary signaling domain is triggered by, for example, the binding of TCR/CD3 complex to peptide loaded MHC molecules, thereby mediating T cell responses (including but not limited to proliferation, activation, differentiation, etc.). The primary signaling domain, which functions in a stimulatory manner, can comprise an immunoreceptor tyrosine activation motif or a signaling motif of ITAM. For example, fragments comprising the primary signaling domain of ITAM include, but are not limited to, the intracellular signaling domains of CD3γ, CD3δ, CD3ε, CD3ζ, CD5, CD79a, CD79b, CD278, and CD66d.

The term “signaling domain (or signal transduction domain)” refers to a functional part of a protein that functions by transmitting information within the cell and is used to regulate the activity of the cell through a defined signaling transduction pathway by producing a second messenger or by acting as an effector in response to such a messenger. The intracellular signaling domain may comprise the entire intracellular portion of the molecule, or the entire natural intracellular signaling domains, or functional fragments or derivatives thereof.

The term “costimulatory signaling domain” or “costimulatory molecule”: usually refers to the intracellular domain of costimulatory molecules that can combine with cell stimulating signal molecules, such as TCR/CD3, to induce T cell proliferation and/or upregulation or downregulation of key molecules. Costimulatory molecules are usually associated conjugative partners on T cells, which specifically bind to costimulatory ligands and mediate the costimulatory response of T cells, including but not limited to proliferation. Costimulatory molecules are non antigen receptor cell surface molecules or their ligands required for an effective immune response. Costimulatory molecules include, but are not limited to, MHC class I molecules, BTLA and TOLL ligand receptors, as well as OX40, CD2, CD27, CD28, CDS, ICAM-1, LFA-1 (CD11a/CD18) and 4-1BB (CD137).

The term “immunosuppressive signaling domain” or “immunosuppressive receptor molecule”: usually refers to the receptor that can negatively regulate and inhibit the activation signal of immune cells, such as the classical ITIM domain comprising receptor. It also comprises non classical ITIM domains, such as the CD300A receptor. It also comprises other ITIM independent inhibitory receptors or enzyme molecules, such as CTLA-4, IDO1 and IDO2.

The term “CD3ζ (also known as CD3 zeta)” comprises the protein provided by GenBank accession number BAG36664.1, or comprises equivalent residues of non-human species such as mice, rodents, monkeys, apes, etc. In the present application, “CD3ζ“is interchangeable with “CD3z” and “CD3Z”.

The term “T cell receptor (TCR)” mediates the recognition of specific major histocompatibility complex (MHC)-restricted peptide antigens by T cells, including classical TCR receptors and optimized TCR receptors. The classical TCR receptor consists of two peptide chains, a and J, each of which can be divided into a variable region (V region), a constant region (C region), a transmembrane domain and a cytoplasmic region. Its antigen specificity exists in the V region. Each of the V region (Vα, Vβ) has three hypervariable regions CDR1, CDR2 and CDR3 respectively. In one aspect, T cells expressing the classical TCR can induce the TCR of T cells to respond specifically to target antigens by means of antigen stimulation to T cells.

The term “engineered” or “engineering” refers to using the principles and methods of cell biology and molecular biology to change the genetic substance in cells or obtain cell products according to people's wishes at the level of the whole cell or organelle through certain engineering means. For example, “engineered” refers to one or more changes in nucleic acids, such as those in the genome of an organism. “Engineered” can refer to changes, additions, and/or deletions of genes. “Engineered cells” can also refer to cells with added, deleted, and/or altered genes.

In one example, the engineered cells are immune cells, neurons, epithelial cells, endothelial cells, or stem cells. Stem cells comprise human pluripotent stem cells (including human induced pluripotent stem cells (iPSCs) and human embryonic stem cells). For example, the engineered cells are immune cells. For example, the engineered cells are primary cells. For example, the engineered cells are B cells, monocytes, natural killer cells, basophils, eosinophils, neutrophils, dendritic cells, macrophages, T cells, NKT cells, or combinations thereof. The engineered cells can be autologous cells or allogeneic cells. For example, the engineered cells are obtained by genetically engineered T cells isolated from human PBMC cells.

The term “immune cell” refers to the cells that participate in the immune response and produce immune effects, such as T cells, B cells, natural killer (NK) cells, natural killer T (NKT) cells, dendritic cells, CIK cells, macrophages, mast cells, etc. For example, the immune cells are T cells, NK cells, or NKT cells. For example, the T cells can be autologous T cells, xenogeneic T cells, or allogeneic T cells. For example, the NK cells can be autologous NK cells or allogeneic NK cells. For example, immune cells are obtained by sorting donor peripheral blood mononuclear cells (PBMCs). For example, immune cells comprise T cells, NK cells, natural killer T cells (NKT), human embryonic stem cells, or pluripotent stem cells. For example, the method administers the immune cell therapy once, twice, three times or more.

In one example, the interval of continuous administration of the immune cell therapy is at least about 20 days, 21 days, 22 days, 23 days, 24 days, 25 days, 26 days, 27 days, 28 days, 29 days, 30 days, 1.5 months, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or more. For example, the interval of continuous administration of the immune cell therapy is at least about 28 days or more.

The term “T cell” can be PBMC, bone marrow, lymph node tissue, umbilical cord blood, thymus tissue and natural T cells obtained from the site of infection, ascites, pleural effusion, spleen tissue, tumor tissue, or cell populations with specific phenotypic characteristics obtained through sorting, or mixed cell populations with different phenotypic characteristics. For example, “T cell” can be a cell comprising at least one T cell subpopulation: stem cell-like memory T cells (TSCM cells), central memory T cells (TCM), effector T cells (Tef, Teff), regulatory T cells (Tregs) and/or effector memory T cells (TeM). For example, “T cells” can be T cells of a specific subtype, such as αβ T cells or γδ T cells. In certain cases, T cells can be obtained from blood collected from individuals using any number of techniques known to those skilled in the art, such as Ficoll™ isolation and/or apheresis. For example, T cells can be obtained by inducing differentiation of pluripotent stem cells.

For example, cells from circulating blood of an individual are obtained by apheresis. Apheresis product usually comprises lymphocytes, including T cells, monocytes, granulocytes, B cells, other nucleated white blood cells, red blood cells and platelets. For example, cells collected by apheresis can be washed to remove plasma molecules and placed in a suitable buffer or medium for subsequent processing steps. The T cells can from healthy donors or individuals diagnosed with cancer. T cells can be autologous T cells or allogeneic T cells. T cells can be primary T cells. For example, “T cells” can also be T cells carrying chimeric receptors that bind to tumor antigens. For example, CAR T cells, recombinant TCR-T cells.

The term “cell composition” generally refers to a combined form comprising at least two types of cells, in which the first type of cells binds to tumor antigens (e.g., GPC3, CLDN18.2); the second type of cells binds to NK cell markers (e.g., NKG2A). For example, each type of cells may be present in different containers, and may also be formulated into desired formulations simultaneously or separately with appropriate adjuvants when needed; each type of cells can be from different sources (for example, prepared, produced or sold by different manufacturers; for example, natural T cells isolated from donors and T cells derived from stem cells, respectively); each type of cells can be prepared into independent formulations (solid, liquid, gel, etc.); each type of cells can exist in a mixed form. The cell composition may also comprise an effective amount of antibodies, immune conjugates, chimeric receptors, nucleic acids or host cells, and may also comprise a pharmaceutically acceptable carrier.

In one example, the immune cell therapy comprises infusion of a cell composition. The cell composition comprises CAR-T1 cells that bind to tumor antigens and CAR-T2 cells that bind to NK cell markers. For example, the CAR-T1:CAR-T2 dose ratio in the cell composition is approximately equal to 0.5:1, 1:1, 1.5:1, 2:1, 2.5:1, 3:1, 3.5:1, 4:1, 4.5:1, or 5:1.

In one example, the immune cell therapy comprises: administering CAR-T2 cells binding to NK cell markers by intravenous infusion, followed by administering CAR-T1 cells binding to tumor antigens by intravenous infusion with an interval of 30 minutes, 60 minutes, 90 minutes, 120 minutes, 150 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

For example, NK cell markers include but are not limited to: CD2, CD3, CD4, CD5, CD7, CD8, CD16a, CD16b, CD25, CD27, CD28, CD38, CD45, CD48, CD50, CD52, CD56, CD57, CD62L, CD69, CD94, CD100, CD102, CD122, CD127, CD132, CD160, CD161, CD178, CD218, CD226, CD244, CD159a (NKG2A), CD159c (NKG2C), NKG2E, CD314 (NKG2D), CD305, CD335(NKP46), CD337, SLAMF7, TIGIT, or FasL.

For example, NK cell markers are selected from: NKG2 receptor family, killer immunoglobulin like receptor (KIR) family, natural cytotoxicity receptor (NCR), and/or other antigens specifically expressed by NK cells. The NKG2 receptor family comprises NKG2A, NKG2D, and NKG2C. KIR family comprises KIR2DL1, KIR2DL2/3, KIR2DL4, KIR2DL5, KIR3DL1, KIR3DL2, KIR2DS1, KIR2DS2/S3, KIR2DS4, KIR2DS5, or KIR3DS1. NCR comprises NKP30, NKP44, NKP46, or NKp80. Other antigens specifically expressed by NK cells comprise CD159a, CD159c, CD94, CD158, CD56, LIR/ILT2, CD244, CD226, CD2, CD16, CD161, TIGIT, CSI, IL-15R, or FasL.

The term “NK inhibitory receptor (NKIR)” refers to a class of receptors on NK cells that can transduce killing inhibitory signals and inhibit the killing function of NK cells, including HLA specific and non-HLA specific inhibitory receptors. NKIR includes, but is not limited to, immunoreceptor tyrosine-based inhibitory motif (ITIM). For example, NKIR comprises: NKG2/CD94 components, KIR family members, LIR family members, NKR-P1 family members, immune checkpoint receptors, immune checkpoint inhibitors, SIGLEC family members, Ly49 family members, or combinations thereof. For example, NKG2/CD94 components are selected from the group consisting of NKG2A, NKG2C, and CD94. For example, KIR family members are selected from the group consisting of KIR2DL1, KIR2DL2/3, KIR2DL5A, KIR2DL5B, KIR3DL1, KIR3DL2, and KIR3DL3. For example, LIR family members are selected from the group consisting of LIR1, LIR2, LIR3, LIR5, and LIR8. For example, NKR-P1 family members are selected from the group consisting of NKR-P1B and NKR-P1D. For example, immune checkpoint inhibitors comprise: (a) one or more antagonists of checkpoint molecules, which comprise PD-1, PDL-1, TIM-3, TIGIT, LAG-3, CTLA-4, 2B4, 4-1BB, 4-IBBL, A2aR, BATE, BTLA, CD39, CD47, CD73, CD94, CD96, CD160, CD200, CD200R, CD274, CEACAM1, CSF-IR, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E or inhibitory KIR; (b) one or more of atezolizumab, avelumab, durvalumab, ipilimumab, IPH4102, IPH43, IPH33, lirimumab, monalizumab, nivolumab, pembrolizumab, and derivatives or functional equivalents thereof; or (c) at least one of atezolizumab, nivolumab, and pembrolizumab, or one or more of venetoclax, azacitidine, and pomalidomide. For example, immune checkpoint receptors are selected from the group consisting of PD-1, TIGIT, CD96, TIM3, and LAG3. For example, SIGLEC family members are selected from the group consisting of SIGLEC7 and SIGLEC9. For example, Ly49 family members are selected from the group consisting of Ly49A, LY49C, LY49F, LY49G1, and LY49G4.

In the present application, pretreatment is administrated before immune cell infusion. For example, pretreatment is performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before immune effector cell administration. For example, pretreatment is performed with one, two or more chemotherapeutic agents. For example, one microtubule inhibitor is used for pretreatment with one or more other chemotherapeutic agents.

For example, two microtubule inhibitors are used for pretreatment with one or more other chemotherapeutic agents. For example, one microtubule inhibitor such as tubulin polymerization promoter is used for pretreatment with one or more alkylating agents and one or more antimetabolites. For example, one or more paclitaxels are used for pretreatment with one or more alkylating agents and one or more antimetabolites. For example, one microtubule inhibitor (e.g., paclitaxel, especially albumin-bound paclitaxel) is used for pretreatment with two other chemotherapeutic agents (e.g., fludarabine and cyclophosphamide). For the purpose of convenient presentation, the specific implementation of pretreatment with one microtubule inhibitor and two other chemotherapy agents will be described below, taking fludarabine and cyclophosphamide as representative chemotherapy agents, and (albumin-bound) paclitaxel as representative microtubule inhibitors. For example, fludarabine, cyclophosphamide and albumin-bound paclitaxel alone, in pairwise combination or in combination as pretreatment are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, preferably at least 1, 2, 3, 4, 5, 6, 7, 8, 9, 10, 11, 12, 13, 14, 15, 16, 17, 18, 19 or 20 days, more preferably, at least 2, 3, 4, 5, 6, 7 or 8 days, before CAR-T cell infusion. For example, pretreatment comprises the administration of cyclophosphamide. For example, pretreatment comprises administration of cyclophosphamide and fludarabine. For example, pretreatment comprises administration of cyclophosphamide, fludarabine, and albumin-bound paclitaxel.

Fludarabine, cyclophosphamide and albumin-bound paclitaxel can be administered on the same or different days. If fludarabine, cyclophosphamide or albumin-bound paclitaxel is administered on the same day, cyclophosphamide and/or albumin-bound paclitaxel can be administered before or after fludarabine; or fludarabine and/or albumin-bound paclitaxel can be administered before or after cyclophosphamide; or cyclophosphamide and/or fludarabine can be administered before or after albumin-bound paclitaxel.

Fludarabine, cyclophosphamide, and albumin-bound paclitaxel can be administered by any route, including intravenous (IV).

For example, pretreatment comprises the administration of fludarabine not higher than about 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 85, 70, 65, 60, 55, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mg/m²/day; and/or the administration of cyclophosphamide not higher than about 2000, 1500, 1000, 950, 900, 850, 800, 750, 700, 690, 680, 670, 660, 650, 640, 630, 620, 610, 600, 595, 590, 585, 580, 579, 578, 576, 575, 574, 573, 572, 571, 570, 569, 568, 567, 566, 565, 564, 563, 562, 561, 560, 559, 558, 557, 556, 554, 553, 552,551, 550, 549, 548, 547, 546, 545, 544, 543, 542, 541, 540, 539, 538, 537, 536, 535, 534, 533, 532, 531, 530, 529, 528, 527, 526, 525, 524, 523, 522, 521, 520, 519, 518, 517, 516, 515, 514, 513, 512, 511, 510, 509, 508, 507, 506, 505, 504, 503, 502, 501, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20 or 10 mg/m²/day; and/or the administration of albumin-bound paclitaxel about 100 mg/day, about 100 mg/m²/day, or not higher than about 500, 450, 400, 350, 300, 290, 280, 270, 265, 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 49, 48, 47, 46, 45, 44, 43, 42, 41, 40, 35, 30, 25, 20, 15, 10, 5 or 1 mg/m²/day.

The methods described herein may include various other interventions. For example, cyclophosphamide and fludarabine may cause adverse evens in patients after administration. The scope of the present application comprises the administration of components to patients to reduce some of these adverse events. For example, the method involves administering normal saline to patients. Patients can be administered normal saline before or after the administration of cyclophosphamide and/or fludarabine, or before and after the administration of cyclophosphamide and/or fludarabine. For example, patients are administered normal saline before the administration of cyclophosphamide and/or fludarabine on each infusion day and after the administration of cyclophosphamide and/or fludarabine. In addition, adjuvants and vehicles can also be administered to patients, e.g., sodium mesna (sodium 2-mercaptoethane sulfonate). In addition, patients can also be administered exogenous cellular hormones.

Once immune effector cells are administered to a subject (e.g., human), the biological activity of the engineered immune effector cell population is measured by any one of a variety of known methods. Parameters used for evaluation comprise: specific binding of engineered or natural T cells or other immune cells to antigens, in vivo manner (e.g., by imaging) or ex vivo manner (e.g., by ELISA or flow cytometry). The ability of engineered immune effector cells to destroy target cells can be detected by any suitable method known in the art, such as the cytotoxicity test described in the following literature, for example, kochenderfer et al., J. immunotherapy, 32 (7): 689-702 (2009) and Herman et al., J. immunological methods, 285 (1): 25-40 (2004). The bioactivity of immune effector cells can also be measured by measuring the expression and/or secretion of certain cytokines, such as CD10⁷a, IFNγ, IL-2, and TNF. For example, the bioactivity is measured by assessing clinical outcomes, e.g., reduction in tumor burden or load, e.g., assessing the reduction of tumor markers, such as, assessing the toxicity results, persistence, and/or proliferation of cells, and/or the presence or absence of host immune responses.

The term “interval of administration” refers to the time between the administration of multiple treatment courses of immune effector cell therapy to an individual (e.g., including the first administration of the immune cell therapy and the second administration of multiple treatment courses of the immune cell therapy). Therefore, the interval of administration can be indicated as a range. For example, the present application comprises administering individuals multiple treatment courses of the immune cell therapy, and each treatment courses is administered a dose determined by a physician. For example, the specific dose of the immune cell therapy according to the present application can be divided into two or more times, and the total dose administered in divided times is equal to the total dose in the treatment courses determined by the physician.

The term “dose” can be expressed as a dose calculated on the basis of weight or body surface area (BSA). The dose calculated on the basis of weight is the dose administered to patients which is calculated based on the weight of patients, such as mg/kg, number of immune effector cells/kg, etc. The dose calculated on the basis of BSA is the dose administered to patients which is calculated based on the surface area of patients, such as mg/m², and the number of immune effector cells/m².

In one example, when the method described herein comprises multiple treatment courses, the dose of each treatment course is the same. For example, when the method described herein comprises multiple treatment courses, the dose of each treatment course is different. “Divided dose” refers to the single dose administered to the subject in one treatment course when the dose of the whole treatment course is divided into several times. For example, when the dose in one treatment course is divided into multiple doses to be administered to the subject, the divided dose of each administration is the same. For example, when the dose in one treatment course is divided into multiple doses to be administered to the subject, the divided dose of each administration is different. For the purpose of this article, unless otherwise specified, the dose refers to the total amount of immune cells administered or infused in one treatment course.

In one example, a certain total amount of immune cells are infused in one time within the treatment course. For example, a certain total amount of the immune cells are infused in 2, 3, 4, 5, 6, 7, 8, 9, 10 or more times. For example, each infusion of the immune cells is an equal part of the immune cells to be infused. For example, each infusion of the immune cells is a non equal part of the immune cells to be infused. For example, the amount of the immune cells infused each time is determined by the physician according to the specific situation of the subject. The specific situation of the subject can be, for example, the overall health of the subject, the severity of the disease, the response to the previous dose of the same treatment course, the response to the previous treatment course, the combined medication of the subject, the degree or possibility of toxic reactions, complications, cancer metastasis, and any other factors that physicians believe will affect the amount of adoptive cells or immune effector cells suitable for infusion. For example, in the multiple infusions of a certain total amount of immune cells, the amount of immune cells infused each time tends to increase. For example, in the multiple infusions of a certain total amount of immune cells, the amount of immune cells infused each time tends to decrease. For example, in the multiple infusions of a certain total amount of immune cells, the amount of immune cells infused each time tends to increase first and then decrease. For example, in the multiple infusions of a certain total amount of immune cells, the amount of immune cells infused each time tends to decrease first and then increase.

In one example, the interval between two adjacent immune cell therapy administrations is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 80, 81, 82, 83, 84, 85, 86, 87, 88, 89, 90, 91, 92, 93, 94, 95, 96, 97, 98, 99, or 100 days. For example, the interval between two adjacent immune cell therapy administrations is about 1, 2, 3, 4, 5, 6, 7, 8, 9, 10, 11, or 12 months. For example, the interval between two adjacent immune cell therapy administrations is about 1, 2, 3, 4, 5, 6, 7, 8, 9, 10, 11, 12 years or more.

The present application provides a method for treating a subject with GPC3-positive liver cancer. For example, after the local treatment or the local treatment combined with the other adjuvant treatment, GPC3-CAR-T cells are administered by intravenous infusion. For example, after the local treatment or the local treatment combined with the other adjuvant treatment, if there is no imaging recurrence and/or metastasis, the GPC3-CAR-T cells are administered by intravenous infusion. For example, after the local treatment or the local treatment combined with the other adjuvant treatment, if there is no imaging recurrence and/or metastasis, and no enlargement of the other minimal tumor lesions without the local treatment is detected by imaging, the GPC3-CAR-T cells are administered by intravenous infusion. For example, after the local treatment or the local treatment combined with the other adjuvant treatment, if the AFP level in peripheral blood is higher than the upper limit of the normal range, the GPC3-CAR-T cells are administered by intravenous infusion. For example, after the local treatment or the local treatment combined with the other adjuvant treatment, if the AFP level in peripheral blood increases progressively, the GPC3-CAR-T cells are administered by intravenous infusion. For example, after the local treatment or the local treatment combined with the other adjuvant treatment, if the AFP level in peripheral blood is higher than the upper limit of the normal range and increases progressively, the GPC3-CAR-T cells are administered by intravenous infusion. For example, after the local treatment or the local treatment combined with the other adjuvant treatment, if the AFP level in peripheral blood increases progressively and there is no imaging recurrence and/or metastasis, the GPC3-CAR-T cells are administered by intravenous infusion. For example, after the local treatment or the local treatment combined with the other adjuvant treatment, if the level of AFP in peripheral blood increases progressively, there is no imaging recurrence and/or metastasis, and no enlargement of the other minimal tumor lesions without the local treatment is detected by imaging, the GPC3-CAR-T cells are administered by intravenous infusion. For example, after the local treatment or the local treatment combined with the other adjuvant treatment, if the AFP level in peripheral blood is higher than the upper limit of the normal range and increases progressively, and there is no imaging recurrence and/or metastasis, the GPC3-CAR-T cells are administered by intravenous infusion. For example, after the local treatment or the local treatment combined with the other adjuvant treatment, if the AFP level in peripheral blood is higher than the upper limit of the normal range and increases progressively, there is no imaging recurrence and/or metastasis, and no enlargement of the other minimal tumor lesions without the local treatment is detected by imaging, the GPC3-CAR-T cells are administered by intravenous infusion. For example, after the local treatment or the local treatment combined with the other adjuvant treatment, if ctDNA or CTC are detected in peripheral blood, there is no imaging recurrence and/or metastasis, the GPC3-CAR-T cells are administered by intravenous infusion. For example, after the local treatment or the local treatment combined with the other adjuvant treatment, if ctDNA or CTC is detected in peripheral blood, there is no imaging recurrence and/or metastasis, and no enlargement of the other minimal tumor lesions without the local treatment is detected by imaging, the GPC3-CAR-T cells are administered by intravenous infusion. For example, the other adjuvant treatment comprises anti-PD-1, PDL-1, and/or VEGFR antibody therapy.

In one example, the method provided in the present application is used to treat phase IIIa hepatocellular carcinoma (HCC) with recurrence risk after surgical resection. For example, the method provided in the present application is used to treat GPC3-positive phase IIIa hepatocellular carcinoma (HCC) with recurrence risk after surgical resection.

In one example, the method disclosed in the present application is used to treat solid tumors. For example, solid tumors are unresectable tumors. For example, solid tumors are resectable tumors. For example, the local treatment can reduce or eliminate local solid tumors.

In one example, the immune cell therapy is administered as the adjuvant treatment after surgery. For example, after local treatment, the immune cell therapy is administered sequentially with chemotherapy. For example, after surgery, systemic chemotherapy is administered first, followed by the immune cell therapy.

The term “perioperative period” refers to the period surrounding a patient's surgery. For example, the immune cell therapy is administered after the surgery. For example, the other adjuvant treatment comprising the immune cell therapy is administered after surgery.

In one example, the immune cell therapy, or the administration of the adjuvant treatment comprising the immune cell therapy is administered about 6 months, 5 months, 4 months, 3 months, 2 months, 60 days, 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after surgery.

In one example, the tumor is a primary tumor. For example, the tumor is metastatic tumor. For example, the tumor comprises nodular lesions. For example, the tumor is advanced cancers. For example, the tumor does not extend into nearby blood vessels. For example, the tumor is resectable. For example, the tumor extends into nearby blood vessels. For example, the tumor is unresectable. For example, the treatment method of the present application is used to treat unresectable tumors, or reduce the size of unresectable tumors, or alleviate the symptoms of unresectable tumors. For example, the method comprises neo adjuvant treatments with the administration of CAR-T cells. For example, the method comprises the administration of adjuvant treatment comprising the immune cell therapy after surgery, which can reduce the tumor size.

In one example, the adjuvant treatment comprising administration of the immune cell therapy, may be administered to patients with i) unresectable solid tumors; ii) resectable solid tumors; or iii) recurrent refractory tumors. For example, immune cells or pharmaceutical compositions are administered via intratumoral or intravenous routes.

Negative margins are usually observed at the time of surgical resection; however, it is believed that solid tumor recurrence is due to residual micrometastasis that persists after resection, which highlights the potential benefits of the adjuvant treatment in improving solid tumor outcomes.

For example, the method for treating tumors or inhibiting tumor growth comprises: (a) selecting patients with solid tumors at high recurrence risk; (b) administrating surgical resection of solid tumors; and (c) administrating the adjuvant treatment comprising immune cells recognizing the tumor antigen expressed by the solid tumor by intravenous infusion.

For example, the method for treating tumors or inhibiting tumor growth comprises: (a) selecting patients with solid tumors at high recurrence risk; (b) administrating surgical resection of solid tumors; (c) administrating systemic chemotherapy; and (d) administrating the adjuvant treatment comprising administering immune cells recognizing the tumor antigen expressed by the solid tumor by intravenous infusion.

For example, the method for treating tumors or inhibiting tumor growth comprises: (a) selecting patients with solid tumors at high recurrence risk; (b) administrating interventional treatment to reduce or eliminate local tumor lesions; and (c) administrating the adjuvant treatment comprising administering immune cells recognizing the tumor antigen expressed by the solid tumor by intravenous infusion.

For example, the method for treating tumors or inhibiting tumor growth comprises: (a) selecting patients with solid tumors at high recurrence risk; (b) administrating immune cells recognizing the tumor antigen expressed by the solid tumor by intravenous infusion; (c) administrating interventional treatment to reduce or eliminate local tumor lesions; and (d) administrating the adjuvant treatment comprising immune cells recognizing the tumor antigen expressed by the solid tumor by intravenous infusion.

For example, the method for treating tumors or inhibiting tumor growth comprises: (a) selecting patients with solid tumors at high recurrence risk; (b) administrating interventional treatment to reduce or eliminate local tumor lesions; (c) administrating the adjuvant treatments including immune cells recognizing tumor antigens expressed by the solid tumor by intravenous infusion; and (d) administrating interventional treatment to eliminate local tumor lesions.

For example, the liver cancer is resectable. For example, the liver cancer is selected from the group consisting of hepatocellular carcinoma (HCC), fibrolamellar carcinoma, cholangiocarcinoma, angiosarcoma, and hepatoblastoma. For example, the liver cancer is HCC. For example, the liver cancer is recurrent. For example, the liver cancer is metastatic. For example, the patient has liver cancer, and the purpose of surgery is to cure the liver cancer. For example, the patient has a chronic viral infection that has been treated and controlled with antiviral therapy, and the chronic viral infection comprises HIV, HBV, HCV, or a combination thereof. For example, the patient has squamous or non squamous liver cancer. For example, the patient has GPC3 expression in ≥1% of liver cancer cells. As used herein, “liver cancer” refers to cancer of the liver, such as hepatocellular carcinoma, fibrolamellar carcinoma, cholangiocarcinoma, angiosarcoma, and hepatoblastoma. For example, liver cancer comprises hepatocellular carcinoma, hepatic cell carcinoma, hepatoblastoma.

For example, liver cancer is hepatocellular carcinoma (HCC). For example, liver cancer is resectable and recurrent. For example, liver cancer is metastatic. For example, patients are surgical candidates for liver cancer tumor resection. For example, patients have liver cancer, and the purpose of surgery is to cure the liver cancer.

In one example, the administration of CAR-T cell therapy results in one or more of the following: (i) tumor growth and occurrence are delayed in treated subjects compared with untreated subjects or subjects treated with surgical resection alone, for example, tumor growth can be delayed by about 3 days, more than 3 days, about 7 days, more than 7 days, more than 15 days, more than 1 month, more than 3 months, more than 6 months, more than 1 year, more than 2 years, more than 3 years, more than 4 years, more than 5 years, more than 6 years, more than 7 years, or more than 8 years; (ii) disease-free survival (DFS) is improved from the date of surgery until tumor recurrence or death compared with untreated subjects or subjects treated with surgical resection alone; and (iii) overall response rate, complete response, or partial response is improved compared with untreated subjects or subjects treated with surgical resection alone.

In one example, compared with the subject treated with surgical resection alone or surgical resection combined with the other adjuvant treatment, administration of the therapy of the present application to a subject with a solid tumor leads to an improvement in the overall survival (OS) or progression free survival (PFS) of the subject. For example, compared with the subject treated with surgical resection alone or surgical resection combined with the other adjuvant treatment, PFS is increased by at least one month, at least two months, at least three months, at least four months, at least five months, at least six months, at least seven months, at least eight months, at least nine months, at least ten months, at least eleven months, at least one year, at least two years, more than three years, more than four years, more than five years, more than six years, more than seven years, or at least eight years. For example, compared with the subject treated with surgical resection alone or surgical resection combined with the other adjuvant treatment, OS is increased by at least one month, at least two months, at least three months, at least four months, at least five months, at least six months, at least seven months, at least eight months, at least nine months, at least ten months, at least eleven months, at least one year, at least two years, more than three years, more than four years, more than five years, more than six years, more than seven years, or at least eight years.

In the example provided in the present application, the condition of the subject with liver cancer progressed rapidly after receiving local interventional treatment for the tumor. Subsequently, after the same local interventional treatment of tumor, the adjuvant treatment of CAR-T by intravenous infusion significantly reduces the tumor marker level to the normal range. Without further treatment, no tumor is detected in more than 5 years, and the overall survival is more than 8 years. No serious CAR-T cell-related toxicity is found in major organs.

In the example provided in the present application, after receiving local interventional treatment, the condition of the subject with liver cancer progressed rapidly. In the follow-up, after the same local interventional treatment of tumor, and then CAR-T adjuvant treatment by intravenous infusion, the expression level of tumor markers is significantly reduced; if the expression level of tumor markers begins to rise, local treatment can be used to remove the residual primary lesions, and then the expression level of tumor markers decreases to the normal range. Without further treatment, no tumor is detected in more than 5 years, the overall survival is more than 8 years, and no serious CAR-T cell-related toxicity is found in major organs.

The method described herein can be used as the adjuvant treatment, for example, after seeing improvement in the subject from conventional therapy, or when the subject does not respond to conventional therapy.

The method of the present application may be administered when there is little evidence of cancer but there is a risk of recurrence. It can be used to kill any cancer cells that have spread to other parts of the body. “Survival” refers to the patient is still alive, including disease-free survival (DFS) and overall survival (OS). “Progression free survival (PFS)” refers to the time from the beginning of randomization to the time when tumor progressed (in any respect) or death (for any reason). For example, PFS comprises SD, PR, or CR. For example, the efficacy evaluation is performed according to RECIST. For example, the efficacy evaluation is performed according to RECIST v1.1. “Disease free survival (DFS)” refers to the survival of patients within a specified period of time without cancer recurrence, such as about 1 year, about 2 years, about 3 years, about 4 years, about 5 years, or about 10 years, from the beginning of treatment or from the initial diagnosis. In the basic research of the present application, DFS is analyzed according to the intention to treat principle, that is, patients are evaluated based on their assigned treatment. Events used in DFS analysis comprise local, regional, and distant recurrence of cancer, occurrence of secondary cancer, and death from any cause in patients without previous events. “Overall survival” refers to the survival of patients within a specified period of time from the start of treatment, or from the initial diagnosis, such as about 1 year, about 2 years, about 3 years, about 4 years, about 5 years, or about 10 years.

The “hazard ratio” in the survival analysis is a summary of the difference between the two survival curves, indicating the reduction of the risk of death after treatment over a period of time compared with the control group. Hazard ratio is the statistical definition of event rate. “Cancer recurrence” herein refers to the recurrence of cancer after treatment, and comprises the recurrence and distant recurrence of cancer.

Subjects at “high cancer recurrence risk” are those with a greater chance of experiencing cancer recurrence, e.g., cancers with vascular invasion, positive distant lymph nodes, the presence of distant metastasis, the presence of minimal residual disease (MRD), or the maintenance of high levels or progressive elevation of tumor markers. The risk level of a subject can be determined by a skilled physician.

Herein, “cure” of cancer refers to the absence of cancer recurrence for about 4 or 5 years after starting the adjuvant treatment.

The cells are separated from peripheral blood mononuclear cells (PBMCs) or T cells of human subjects with cancer based on “monocyte isolation technique”, and cultured and transduced with viral vectors encoding chimeric antigen receptors (CARs), which specifically bind to antigens expressed by cancer in subjects, which are tumor associated or tumor specific antigens. The cells are cryopreserved in infusion media in separate flexible cryobags, each comprising a single unit dose of cells ranging from approximately 1×10⁶ cells to 5×10⁷ cells. The total dose of each infusion of immune cell therapy is not higher than about 1×10¹² cells, preferably, not higher than about 1×10¹¹ cells, more preferably, not higher than about 1×10¹⁰ cells or about 5×10′ cells or about 2×10⁹ cells. Before infusion, the cells are maintained at a temperature below approximately −130° C. or approximately −175° C.

Before starting cell therapy, blood is obtained from subjects, and the levels of one or more serum factors indicative of cytokine release syndrome (CRS), such as tumor necrosis factor alpha (TNFα), interferon gamma (IFN γ), IL-10, and IL-6 in serum, are optionally assessed by ELISA and/or MSD and/or CBA methods. Before starting treatment, tumor burden can optionally be assessed by measuring the size or quality of solid tumors, for example, by PET or CT scan.

Resuscitation is performed by heating up to approximately 38° C., and the subject is administered the first dose of cells by multiple infusions. Each infusion is administered by continuous intravenous (IV) infusion for about 3-30 minutes.

After administering the immune cell therapy, the subject receives physical examination and is monitored for any symptoms of toxicity or toxic results, such as fever, hypotension, hypoxia, neurological disorders, or elevated serum levels of inflammatory cytokines or C-reactive protein (CRP). Optionally, after administering the immune cell therapy, blood is obtained from patients once or multiple times, and the levels of serum factors indicative of CRS are assessed by ELISA and/or MSD and/or CBA methods. The levels of serum factors are compared with those obtained just before the first dose is administered. If necessary, anti-IL6 or other CRS treatments are administered to reduce the symptoms of CRS.

After the administration of the immune cell therapy, such as 1, 2, 3, and/or 4 weeks after the start of administration, the presence or absence of anti-CAR immune response in the subject is optionally detected, for example, by qPCR, ELISA, ELISPOT, cell-based antibody assay, and/or mixed lymphocyte reaction.

The percentage reduction in tumor burden achieved by the immune cell therapy can optionally be measured one or more times after the first dose is administered in patients with solid tumors by scan (e.g., PET and CT scan), and/or by quantifying disease positive cells in the blood or tumor site (e.g., CTC, ctDNA).

Subjects are monitored regularly from the beginning of the immune cell therapy and continued for up to several years. During follow-up, tumor burden is measured, and/or CAR-expressing cells are detected by flow cytometry and quantitative polymerase chain reaction (qPCR) to measure the in vivo proliferation and persistence of the administered cells, and/or to evaluate the development of anti-CAR immune responses.

In one example, the selection criteria for the trial population of the present application: patients with advanced tumors who have failed in the treatment of various types of tumors (such as chemotherapy, radiotherapy, and surgical resection).

In one example, if patients with gastric or gastroesophageal junction (G/GEJ) adenocarcinoma with CLDN18.2-positive tumor cells accounting for >=10% can not tolerate conventional chemotherapy after surgery, or the risk of continuing adjuvant chemotherapy is greater than the benefit, the CLDN18.2-CAR-T cells are administered. The conventional chemotherapy is the standard postoperative adjuvant chemotherapy according to the diagnosis and treatment guidelines, e.g., CapeOX, FOLFOX, FLOT, etc.

In one example, if patients with pancreatic cancer with CLDN18.2-positive tumor cells accounting for >=10% can not tolerate conventional chemotherapy after surgery, or the risk of continuing adjuvant chemotherapy is greater than the benefit, the CLDN18.2-CAR-T cells are administered. The conventional chemotherapy is the standard postoperative adjuvant chemotherapy according to the diagnosis and treatment guidelines, e.g., gemcitabine+capecitabine, gemcitabine+albumin-bound paclitaxel or mFOLFIRINOX.

In one example, the total infusion dose of the GPC3-CAR-T treatment is 5×10⁸ cells/time. For example, GPC3-CAR-T pretreatment: fludarabine is administered by intravenous infusion at 25 mg/m²/day on D-5, D-4, and D-3; and cyclophosphamide is administered by intravenous infusion at 300 mg/m²/day on D-5, D-4, and D-3.

In one example, the total infusion dose of the CLDN18.2-CAR-T treatment is 1×10⁸ cells/time, 2.5×10⁸ cells/time, 4×10⁸ cells/time, or 5×10⁸ cells/time. For example, CLDN18.2-CAR-T pretreatment: fludarabine is administered by intravenous infusion at 25 mg/m²/day on D-5, and D-4; and cyclophosphamide is administered by intravenous infusion at 250 mg/m²/day on D-5, D-4, and D-3; albumin-bound paclitaxel is administered by intravenous infusion at 100 mg m²/day on D-4.

In one example, CLDN18.2-CAR-T pretreatment: fludarabine is administered by intravenous infusion at 30 mg/m²/day on D-5, D-4, and D-3; cyclophosphamide is administered by intravenous infusion at 250 mg/m²/day on D-5, D-4, and D-3; and albumin-bound paclitaxel is administered by intravenous infusion at 100 mg m²/day on D-4.

In one example, the method provided in the present application is used to treat HCC first diagnosed as CNLC phase IIIa, in which preoperative imaging detection reveals the presence of any one of the following vascular cancer thrombus, and without atrial cancer thrombus: portal vein cancer thrombus (PVTT); hepatic vein tumor thrombus (HVTT); inferior vena cava tumor thrombus (IVCTT). For example, surgical resection has been performed: the pathological evaluation of surgical resection specimen shows a negative margin; the neo adjuvant treatment and/or postoperative treatment are allowed. For example, the participant has recovered from liver resection surgery, and has shown a progressive increase in AFP levels (including: 1 AFP increased by at least 20% at any 3 months after surgery; or 2. AFP increased by >=10% for any two consecutive times after surgery) after the surgery, which has been assessed by the researchers as having a potential recurrence tendency. The expression intensity of GPC3 in tumor tissue specimen is >=1+ and the percentage of positive tumor cells is >=10% by IHC.

In one example, the present application provides a method for treating patients with gastric or gastroesophageal junction (G/GEJ) adenocarcinoma. In one example, patients with gastric or gastroesophageal junction (G/GEJ) adenocarcinoma are administered the immune cell therapy after surgery. In one example, patients with gastric or gastroesophageal junction (G/GEJ) adenocarcinoma are administered the immune cell therapy after surgery, followed by the adjuvant treatment.

The present application is further described below in combination with specific examples. It should be understood that these Examples are only used to illustrate the present application and not to limit the scope of the present application. The experimental methods without specific conditions indicated in the following examples are usually based on the conventional conditions, such as the conditions described in the guide to molecular cloning experiments, Third Edition, Science Press, 2002, compiled by J. Sambrook, etc., or the conditions recommended by the manufacturer.

### EXAMPLES

### Example 1. Preparation of CAR-T Cells

GPC3-CAR-T cells were prepared according to WO2018018958A1. CLD18A2-CAR-T cells were prepared according to WO2019170147A1.

In brief, a vector expressing the chimeric receptor disclosed in the present application was constructed by using conventional molecular biology techniques. DNA was synthesized, and the CAR targeting GPC3 or the CAR targeting CLD18A2 were constructed and inserted into the MluI and SalI restriction endonuclease sites of the pRRLSIN lentiviral vector (Addgene), respectively, for lentiviral packaging. GPC3-CAR or CLD18A2-CAR successively comprise sequences as shown in SEQ ID NO:28, or 51. The corresponding lentivirus of the above expression vectors were prepared by conventional molecular biology technology.

The conventional method for preparing CAR-T in the art was used: PBMC cells from healthy donors were collected and activated with magnetic beads (LIFE TECHNOLOGIES, 40203D) with anti-CD3 and CD28 antibodies. The T cells were infected with lentivirus comprising the above vectors GPC3-CAR or CLD18A2-CAR to obtain the corresponding CAR-T cells. T cells that were not transfected with virus were identified as UTD. The positive rate of CAR was detected by FACS.

### Example 2: Treatment with CAR-T Cells

For the subjects with recurrent, refractory, or distant metastasis of solid tumors were systemically administered CAR-T therapy after receiving local treatment to reduce or eliminate the tumo. Local treatment to eliminate tumors includes but is not limited to surgical resection of tumors, cryoablation of tumors, microwave ablation of tumors, vascular embolization of tumors, local radiotherapy of tumors, or a combination thereof.

Before CAR-T cells were administered, patients received apheresis technology of “de mononuclear cell isolation”, and underwent pretreatment.

PBMCs were obtained from the subjects by apheresis, and CAR-T cells were obtained by transduction and expansion with viral vectors encoding CARs that recognize tumor antigens. The resulting CAR-T cells were frozen and stored in cryosolution using a cryobag.

Before CAR-T cell therapy was administered, tumor burden can be optionally assessed by measuring the size or characteristics of solid tumors, using such as PET or CT scan, and tumor burden can also be assessed by detecting tumor markers and/or observing the occurrence and severity of tumor complications.

Subjects were pretreated 2-10 days before CAR-T cell infusion. Pretreatment comprised: administering cyclophosphamide to the subject; administering fludarabine and cyclophosphamide to the subject; or administering fludarabine, cyclophosphamide and albumin-bound paclitaxel to the subject.

When the subject required multiple cycles of CAR-T cell therapy, the CAR-T cells in the subsequentcycle were administered 4 weeks after the completion of CAR-T administration in the previous cycle. The CAR T cell administration in each cycle can be administered once, or divided into twice or more times of intravenous (IV) infusions. Each infusion took about 3-30 minutes to complete the infusion, preferably 5-25 minutes to complete the infusion.

After CAR-T cell administration was completed in each cycle, the subject would undergo physical examination and be monitored for any symptoms of toxicity or toxic results, such as fever, hypotension, hypoxia, neurological disorders or elevated serum levels of inflammatory cytokines or C-reactive protein (CRP). The examination can be performed by obtaining blood from patients to assess the cytokine levels indicating CRS by ELISA, and/or MSD, and/or CBA. If necessary, anti-IL6 treatment was administered, or other CRS treatments were administered to reduce the symptoms of CRS.

After CAR-T cell administration was completed in each cycle, for example, 1, 2, 3, and/or 4 weeks after the start of CAR-T cells administration, the number of CAR-T cells in the subject can be assessed by qPCR, ELISA, ELISPOT, antibody assay, etc.

The reduction of tumor burden after each cycle of treatment can be obtained by scanning (such as PET and CT scan), and/or by quantifying the number of cells that were positive for tumor antigens (such as claudin18.2, GPC3, HER2) in the blood or tumor sites.

### Example 3: Assessment of Treatment

The tumor burden was assessed, and the number and size of tumor lesions were determined by imaging detection of target lesions and non-target lesions before and after treatment. The criteria for efficacy evaluation is described in RECIST v1.1 for solid tumor efficacy evaluation.

All adverse events (AES) would be classified according to code of the latest version of the ICH International Dictionary of medical terms MedDRA (Medical Dictionary for Regulatory Activities), and according to the Common Terminology Criteria for Adverse Events (CTCAE V5.0 classification, using frequency distribution, charts or other descriptive indicators for analysis, the number and percentage of subjects with adverse events after treatment would be calculated according to the system organ classification, preferred terms and groups).

### Example 4. Patients with Liver Cancer were Treated with CAR-T by Intravenous Infusion after the Local Tumor Treatment

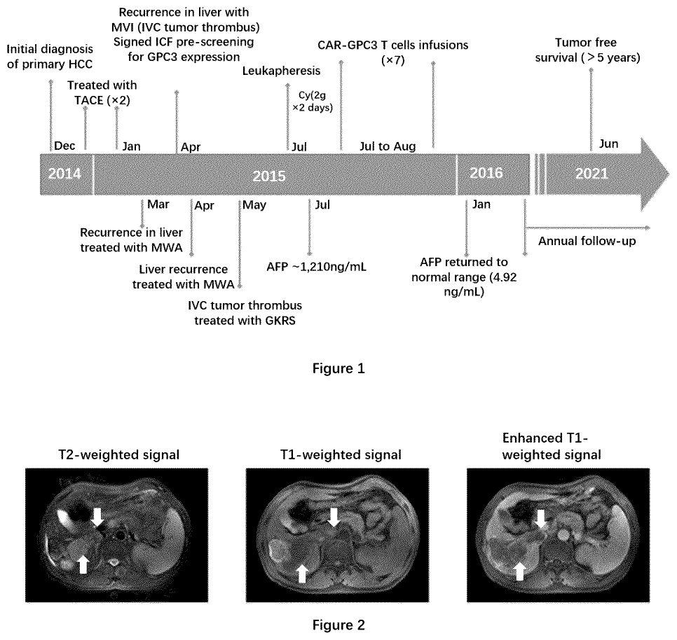

Subject 1, a 50 years old male with hepatitis B cirrhosis, was diagnosed with stage IB HCC according to the China Liver Cancer Staging (CNLC) in December 2014, and the liver function grade was Child-Pugh A. After achieving partial response (PR) with transcatheter arterial chemoembolization (TACE), the underwent microwave ablation (MWA) in March 2015. However, the patient's condition progressed rapidly, and inferior vena cava tumor thrombus (IVCTT) occurred within 6 weeks after MWA treatment (FIGS. 1, and 2).

The subject was enrolled in this clinical trial in April 2015. The subject first received local treatment: MWA for recurrent liver cancer lesions and gamma knife radiotherapy (GKRS) for IVCTT (FIGS. 1, 3).

84 days after local treatment, GPC3-CAR-T cells were administered by intravenous infusion, with a total dose of about 7.10×10⁸ CAR-GPC3-T cells. Pretreatment was administered before CAR-T cell infusion: cyclophosphamide 2000 mg/day×2.

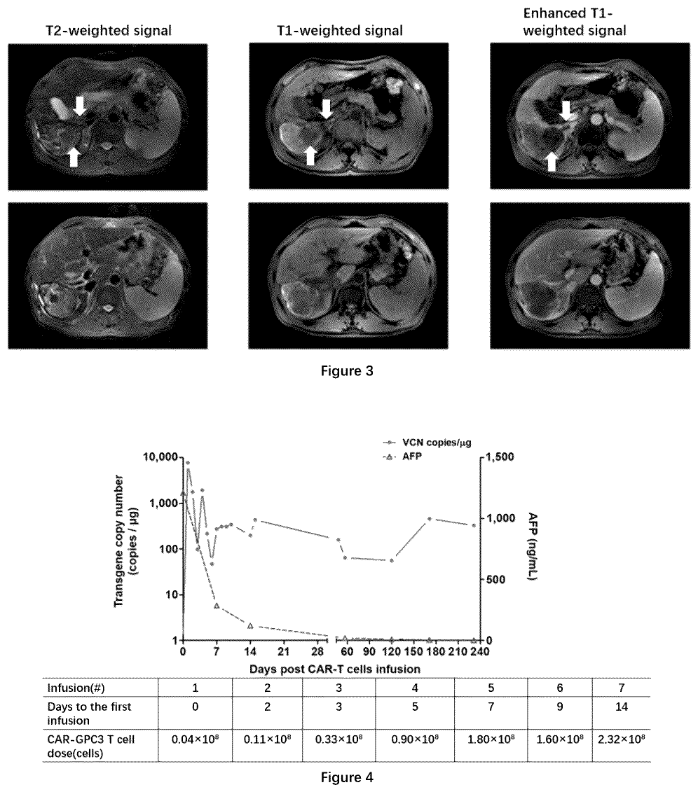

Alpha fetoprotein (AFP) was detected to be about 1210 ng/ml before CAR-T infusion., which rapidly decreased to about 121 ng/ml on the 14th day after CAR-T infusion, and gradually returned to the normal range within the next two months (FIG. 4).

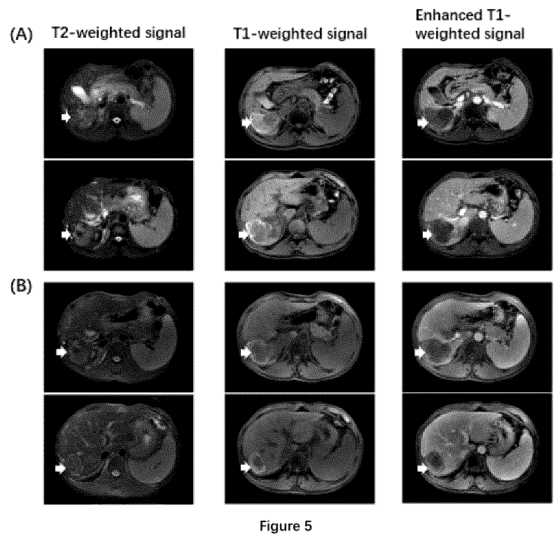

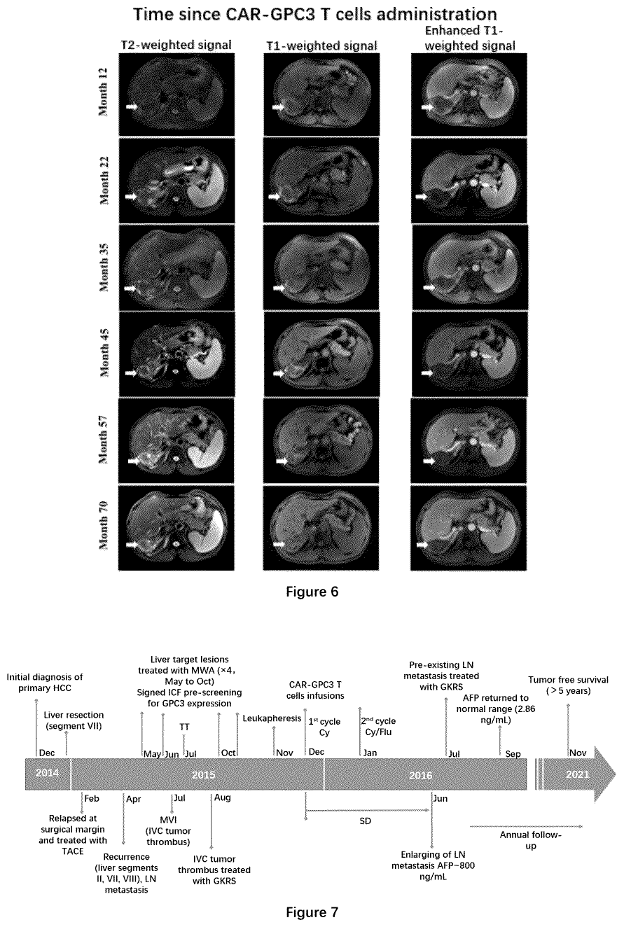

The subjects did not experience tumor recurrence after receiving local treatment and CAR-GPC3-T cell therapy (FIGS. 5, and 6). No tumor was detected in more than 5 years without further treatment, the overall survival was more than 8 years, and the AFP level was normal. No CAR-GPC3-T cell related toxicity was observed in major organs.

### Example 5. Patients with Liver Cancer were Treated with Multiple Cycles of CAR-T by Intravenous Infusion after the Local Tumor Treatment

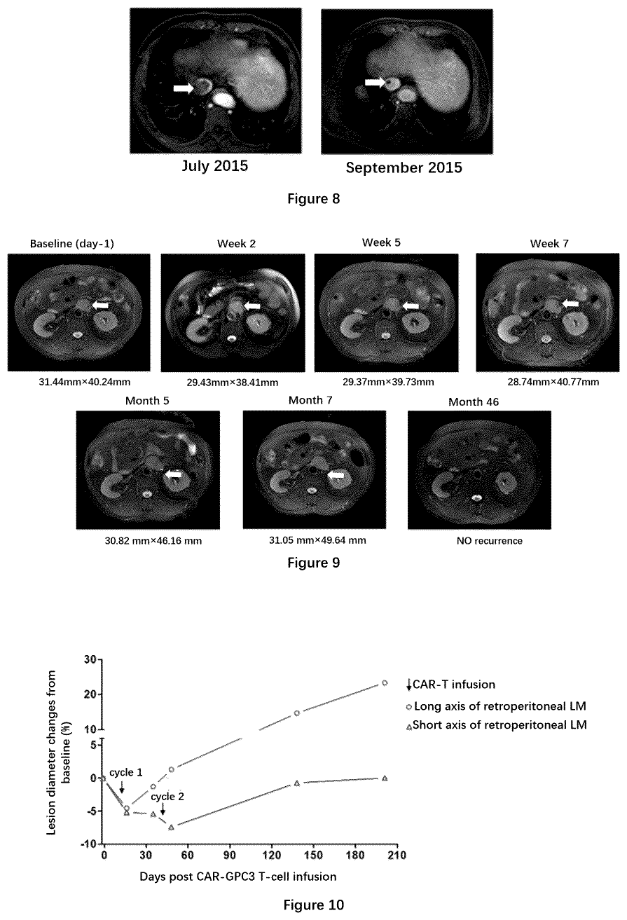

Subject 2, a 54 years old male with hepatitis B cirrhosis, was diagnosed with stage IB HCC according to CNLC and underwent surgical resection of the tumor in December 2014. Six weeks after surgery, the tumor recurred at surgical incision edge in February 2015. Then, from February to June 2015, once TACE was performed to treat the recurrent tumor at the surgical incision edge, and twice MWA were performed to treat IVCTT. In July 2015, magnetic resonance imaging (MRI) showed rapidly disease progression with multifocal lesions in the liver, IVCTT (FIGS. 7, and 8), and retroperitoneal lymphatic metastasis (LM).

The subject received GKRS for IVCTT in August 2015 and twice MWA for multifocal lesions of liver cancer in October 2015 (FIG. 7). The inferior vena cava tumor thrombus was basically absorbed after local treatment.

49 days after local treatment, the first GPC3-CAR-T cell treatment was administered, with a total dose of 40.7×10⁸ GPC3-CAR-T cells. Two days before CAR-T cell infusion, pretreatment was administered: cyclophosphamide 2000 mg/day×1. Two weeks after infusion, the size of retroperitoneal LM began to decrease; the short axis diameter of the lesion decreased by about 5.2%, and the long axis diameter decreased by about 4.5%.

89 days after local treatment, the second GPC3-CAR-T cell treatment was administered, with a total dose of 11.09×10⁸ GPC3-CAR-T cells. Three days before CAR-T cell infusion, pretreatment was administered: cyclophosphamide 2000 mg/day×1, fludarabine 50 mg/day×1.

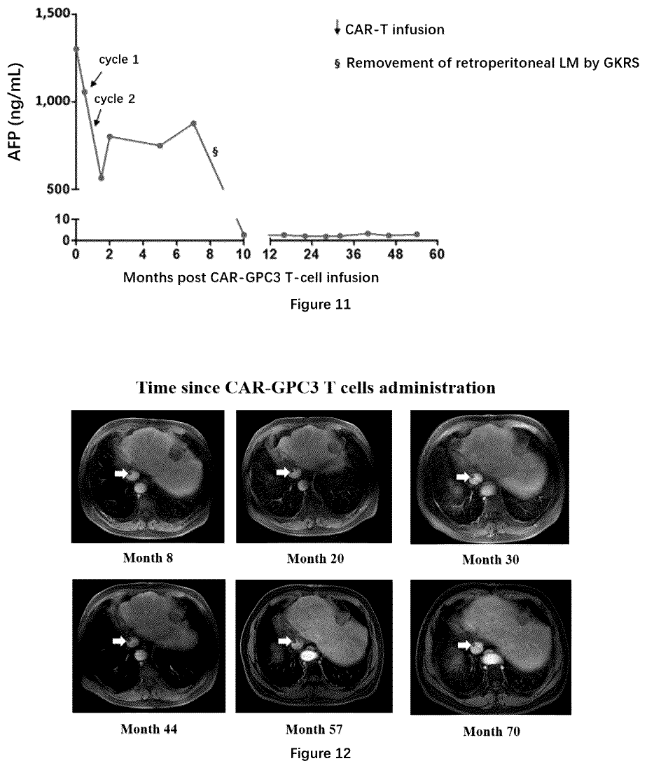

After 2 cycles of GPC3-CAR-T cell treatment, the short axis of the target lesion further reduced (FIG. 10); the AFP level decreased by about 56.6% (from about 1301 ng/ml to about 565 ng/ml) (FIG. 11). The efficacy was assessed as SD according to RECIST v1.1 for 7 months.

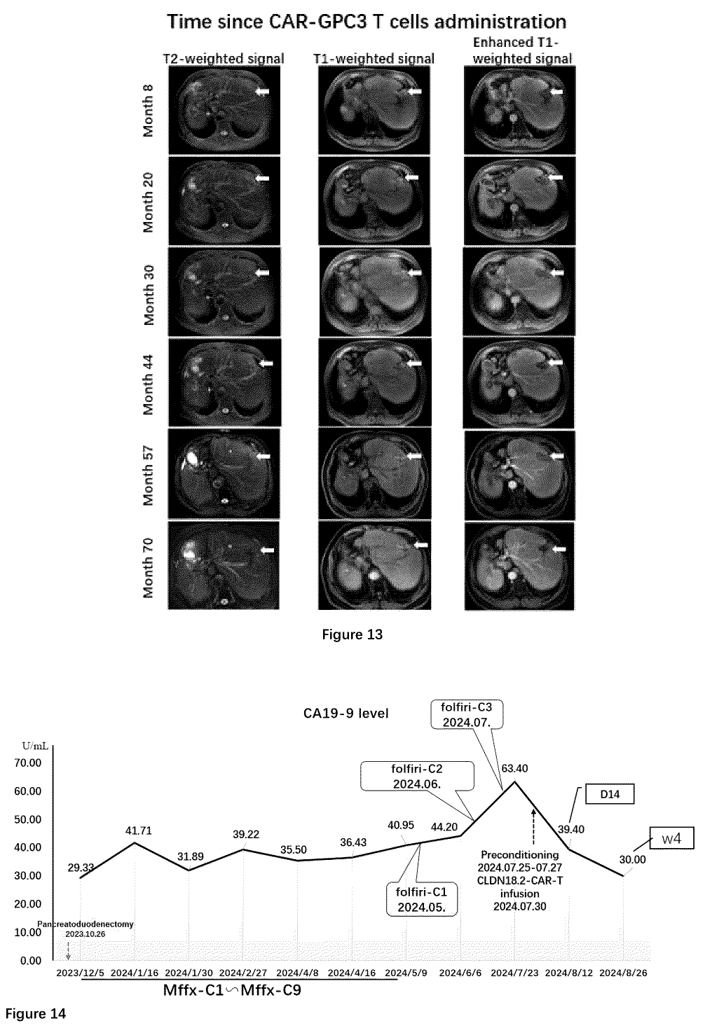

In June 2016, AFP elevation was detected, MRI showed enlarged retroperitoneal LM (FIG. 9), and GKRS was performed to remove retroperitoneal LM. After surgery, AFP gradually decreased to the normal range and was in a cancer-free state (FIGS. 12, and 13). Without any further anticancer treatment, the patient remained disease-free for more than 5 years, and the overall survival was more than 8 years. Fever, fatigue, transient leukopenia, thrombocytopenia, and grade 1 cytokine release syndrome were reported during GPC3-CAR-T cell infusion.

### Example 6. Patients with Liver Cancer Received at Least One Cycle of CAR-T Treatment by Intravenous Infusion after Radical Resection of Liver Cancer

Patients with liver cancer received at least once administration of GPC3-CAR-T by intravenous infusion after radical resection of liver cancer. PD-L1 antibody or Avastin can also be administered as adjuvant treatments.

For example, patients with liver cancer received GPC3-CAR-T by intravenous infusion after radical resection of liver cancer. Pretreatment protocol was administered before GPC3-CAR-T infusion: fludarabine 25 mg/m²/day, cyclophosphamide 300 mg/m²/day, intravenous drip.

If the patient was in non-progressive stage (e.g., SD, PR) after efficacy evaluation, the second administration of GPC3-CAR-T cells by intravenous infusion can be performed at an interval of about 28 days. GPC3-CAR-T cells can also be continuously infused for multiple times until disease progression or complete remission (CR).

The key criteria for the selection of subjects were as follows: HCC patients aged 18-75 years (including) with GPC3-positive tumor cells and at recurrence risk after radical resection of liver cancer.

### Example 7. Patients with Pancreatic Cancer were Treated with CAR-T by Intravenous Infusion after Radical Resection of Pancreatic Cancer

CLDN18.2-CAR-T was infused intravenously after radical resection of pancreatic cancer in patients with pancreatic cancer.

For example, CLDN18.2-CAR-T was infused intravenously after radical resection of pancreatic cancer in patients with pancreatic cancer. If the patient was in non-progressive stage (e.g., SD, PR) after efficacy evaluation, the second infusion of CLDN18.2-CAR-T cells can be performed at an interval of about 28 days. CLDN18.2-CAR-T cells can also be administered continuously for multiple rounds until disease progression or complete remission (CR).

The key criteria for the selection of subjects were as follows: pancreatic cancer patients aged 18-79 years (including) with CLDN18.2-positive tumor cells and at risk of recurrence after radical resection of pancreatic cancer.

### Example 8. Patients with Pancreatic Cancer were Treated with CAR-T by Intravenous Infusion after Radical Resection of Pancreatic Cancer and Adjuvant Chemotherapy

After radical resection of pancreatic cancer, patients with recurrence risk after adjuvant chemotherapy were administered CLDN18.2-CAR-T cell therapy. Adjuvant chemotherapy comprised gemcitabine+capecitabine, gemcitabine+albumin-bound paclitaxel, or mFOLFIRINOX.

The key criteria for subject selection were as follows: patients aged 18-79 years; histologically confirmed pancreatic cancer; the tumor cells were positive for CLDN18.2, and the tumor was completely resected observed by naked eye, and postoperative pathological stage (pTNM): T1-3, NO-2, MO. Immunohistochemical (IHC) staining of tumor tissue samples showed that CLDN18.2 was positive (expression intensity ≥2+ and the percentage of positive tumor cells ≥40%).

The subject, a 59 years old female, underwent radical pancreatoduodenectomy and intestinal adhesiolysis. Postoperative pathology: moderately differentiated ductal adenocarcinoma of the pancreas, 3*2*2 cm, with cancer tissue infiltrating into the pancreatic stump margin and common bile duct wall, nerve invasion, no vessel carcinoma embolus, no tumor cell involvement in gastric margin, duodenal margin, bile duct margin and omental tissue, no cancer tissue was found in 4 perigastric lymph nodes (0/4), and cancer tissues were found in 2 of the 3 peripancreatic lymph nodes(2/3).

After the surgery, the tumor marker CA19-9 was maintained at 29-41 U/ml during 9 cycles of chemotherapy with Mfolfininox (Mffx) protocol for about 6 months; and then 3 cycles of folfiri chemotherapy was performed, the tumor marker CA19-9 began to rise to 63.4 U/ml (FIG. 14), and no new lesions were detected by imaging.

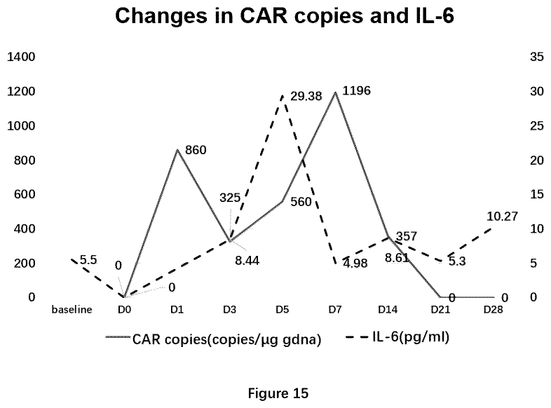

After the pretreatment protocol, 5×10⁸ CLD18A2-CAR-T cells were infused intravenously (recorded as day DO). The pretreatment protocol was as follows: fludarabine were administered at 30 mg/m²/day on D-5, D-4, D-3, intravenous drip; cyclophosphamide were administered at 250 mg/m²/day on D-5, D-4, D-3, intravenous drip; albumin-bound paclitaxel was administered at 100 mg/day on D-4, intravenous drip. CA19-9 decreased after CAR-T infusion: D14:39.4 U/ml; W4 (day 28 after CAR-T infusion): 30 U/ml (FIG. 14), and no new lesions were detected by imaging. In vivo CAR-T copy number changes were shown in FIG. 15.

The Examples described in the present application comprise taking this Example as any single Example or combining it with any other Examples or part thereof. In addition, it should be understood that after reading the above teaching contents of the present application, those skilled in the art can make various changes or modifications to the present application, and these equivalent forms also fall within the scope of the claims attached to the present application.

1. An adjuvant treatment method, comprising administering the adjuvant treatment to a subject with a solid tumor after receiving local treatment; wherein the adjuvant treatment comprises: administering a therapeutically effective amount of immune cell therapy comprising a chimeric receptor to the subject by intravenous infusion.

2. The method according to claim 1, wherein the local treatment reduces or eliminates tumor burden;
preferably, wherein the local treatment comprises: surgical treatment, cryoablation, microwave treatment, vascular embolization, radiofrequency treatment, gamma knife treatment, focused ultrasound treatment, photodynamic treatment, argon-helium knife treatment, radioactive particle implantation, or any combination thereof;
more preferably, wherein the surgical treatment comprises radical tumor surgery or palliative tumor surgery.

3. The method according to claim 1, wherein the immune cell therapy is administered after the local treatment combined with other adjuvant treatment; and the other adjuvant treatment does not comprise the immune cell therapy;
preferably, wherein the other adjuvant treatment comprises chemotherapy, radiotherapy, hormone therapy, immune checkpoint inhibitor therapy, immune modulator therapy, antibody therapy, antiangiogenic drug therapy, small molecule compound therapy, or any combination thereof;
more preferably, wherein the chemotherapy is systemic chemotherapy.

4. The method according to claim 1, wherein after the local treatment or the local treatment combined with the other adjuvant treatment, the immune cell therapy is administered when no tumor recurrence and/or metastasis is detected; or wherein after the local treatment or the local treatment combined with the other adjuvant treatment, the immune cell therapy is administered if no enlargement of other small tumor lesions without the local treatment is detected by imaging technology.

5. The method according to claim 1, wherein the chimeric receptor binds to the tumor antigen expressed by the solid tumor.

6. The method according to claim 1, wherein after the local treatment or the local treatment combined with the other adjuvant treatment, the immune cell therapy is administered if the tumor marker level is detected to be higher than the upper limit of the normal range or higher than the baseline level, or the tumor marker level is detected to be progressively increased; or wherein after the local treatment or the local treatment combined with the other adjuvant treatment, the immune cell therapy is administered if circulating tumor DNA (ctDNA) or a circulating tumor cell (CTC) are detected in peripheral blood; or wherein after the local treatment or the local treatment combined with the other adjuvant treatment, the immune cell therapy is administered if the subject is in progression free survival (PFS),
preferably, wherein the efficacy is evaluated with reference to RECIST to determine whether the subject is in progression free survival (PFS).

7. The method according to claim 1, wherein the chimeric receptor comprises a chimeric antigen receptor (CAR) and/or a recombinant TCR;
preferably, wherein the chimeric receptor comprises at least one, two or three intracellular signaling domains;
more preferably, wherein the intracellular signaling domain comprises a signaling domain of CD3, CD28, 4-1BB, OX40, DAP10 or ICOS.

8. The method according to claim 1, wherein the immune cell comprises: a T cell, a NK cell, a natural killer T cell (NKT), a human embryonic stem cell, or a pluripotent stem cell;
preferably, wherein the immune cell is an autologous or allogeneic cell.

9. The method according to claim 1, comprising administering the immune cell therapy once, twice or more than three times;
preferably, wherein when administered 2 or 3 or more times, the interval between adjacent immune cell therapy administrations is at least about 28 days or more.

10. The method according to claim 1, wherein pretreatment is administered about 1-12 days before each administration of the immune cell therapy;
preferably, wherein the pretreatment comprises administering: cyclophosphamide; a combination of cyclophosphamide and fludarabine; or a combination of cyclophosphamide, fludarabine, and albumin-bound paclitaxel.

11. The method according to claim 4, wherein the imaging detection comprises: computed tomography (CT) scan, bone scan, magnetic resonance imaging (MRI), positron emission tomography (PET), ultrasonic detection, X-ray examination, or any combination thereof.

12. The method according to claim 6, wherein the solid tumor marker comprises: ALK, AFP, B2M, Beta-hCG, BTA, C-kit/CD117, CA15-3, CA19-9, CA724, CA-125, CA 27.29, Calcitonin, CEA, CD19, CD20, CD22, CD25, CD30, CD33, CgA, DCP, ER/PR, 5-HIAA, PSA, SMRP, a squamous cell carcinoma (SCC) antigen, or a soluble fragment of cytokeratin 19 (CYFRA21-1).

13. The method according to claim 1, wherein the tumor antigen expressed by the solid tumor comprises: EGFR or a mutant thereof, B7H3, GPC3, Claudin 6, Claudin18.2, FAP, mesothelin, NKG2D ligand (NKG2DL), NKG2A, CD94, FCRH5, IL13Rα2, GD2, EpCAM, CEA, MUC1, MSLN, PSCA, AFP, or ERBB2.

14. The method according to claim 1, wherein the solid tumor comprises: liver cancer, pancreatic cancer, gastric cancer, esophageal cancer, gastroesophageal junction tumor, colon cancer, rectal cancer, small intestinal cancer, cholangiocarcinoma, gallbladder cancer, lung cancer, laryngeal cancer, kidney cancer, bladder cancer, ovarian cancer, breast cancer, uterine cancer, prostate cancer, gliomas, melanoma, neuroblastoma, sarcoma, or skin cancer.

15. The method according to claim 1, wherein the method is used to treat a subject with a GPC3-positive solid tumor, and after the local treatment or the local treatment combined with the other adjuvant treatment, GPC3-CAR-T cell is administered by intravenous infusion if no tumor recurrence and/or metastasis is detected by imaging technology;
preferably, wherein the GPC3-positive solid tumor comprises: hepatocellular carcinoma, hepatic cell carcinoma, hepatoblastoma, rhabdomyosarcoma, malignant rhabdoid tumor, liposarcoma, Wilms tumor, York sac tumor, embryonal sarcoma of the liver, squamous cell carcinoma of the lung, Merkel cell carcinoma, lung cancer, breast cancer, colo-rectal cancer, or brain tumor;
more preferably, wherein the method is used to treat a subject with GPC3-positive liver cancer, and after the local treatment or the local treatment combined with the other adjuvant treatment, GPC3-CAR-T cell is administered by intravenous infusion if the AFP level in peripheral blood is detected to be higher than the upper limit of the normal range or higher than the baseline level, or the AFP level is progressively increased.

16. The method according to claim 1, wherein the method is used to treat a subject with CLDN18.2-positive solid tumor, and after the local treatment or the local treatment combined with the other adjuvant treatment, CLDN18.2-CAR-T cell is administered by intravenous infusion if no tumor recurrence and/or metastasis is detected by imaging technology;
preferably, wherein the CLDN18.2-positive solid tumor comprises: gastrointestinal tumor, gastric adenocarcinoma, gastroesophageal junction adenocarcinoma, gastric cancer, pancreatic cancer, gastroesophageal junction cancer, esophageal cancer, pancreatic ductal adenocarcinoma, or ovarian carcinoma;
more preferably, wherein the method is used to treat a subject with CLDN18.2-positive gastric cancer or gastroesophageal junction tumor, and after the local treatment or the local treatment combined with the other adjuvant treatment, CLDN18.2-CAR-T cell is administered by intravenous infusion if no tumor recurrence and/or metastasis is detected by imaging technology;
most preferably, wherein the method is used to treat a subject with CLDN18.2-positive pancreatic cancer, and after the local treatment or the local treatment combined with the other adjuvant treatment, CLDN18.2-CAR-T cell is administered by intravenous infusion if the CA19-9 level in peripheral blood is detected to be higher than the upper limit of the normal range or higher than the baseline level, or the CA19-9 level is progressively increased.

17. The method according to claim 1, wherein the antigen binding unit of the chimeric receptor comprises a sequence having at least 80%, 85%, 90%, 91%, 92%, 93%, 94%, 95%, 96%, 97%, 98%, 99% or 100% sequence identity with SEQ ID NO: 1, 2, 3, 4, 5, 6, 7, 8, 47, 48, 49, 50, 62 or 63.

18. The method according to claim 1, wherein the chimeric receptor comprises a sequence having at least 80%, 85%, 90%, 91%, 92%, 93%, 94%, 95%, 96%, 97%, 98%, 99% or 100% sequence identity with SEQ ID NO: 28, 29, 30, 51, 52, 53, 54, 60 or 61.

19. The method according to claim 1, wherein the subject has a high risk of cancer recurrence.

20. An adjuvant treatment method, comprising administering the adjuvant treatment to a subject with a solid tumor after radical surgery for the tumor; wherein the adjuvant treatment comprises: administering a therapeutically effective amount of immune cell therapy comprising a chimeric receptor to the subject by intravenous infusion, and the chimeric receptor binds to the tumor antigen expressed by the solid tumor.


SEQUENCE LISTING (64 sequences)

SEQ ID NO: 1 - protein, 247 aa, synthetic construct
QVQLQESGGGLVQPGRSLRLSCAASGFTFSSYAMHWVRQAPGKGLEWVSAISGSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 2 - protein, 247 aa, synthetic construct
QVQLQESGGGLVQPGRSLRLSCAASGFTFSTYAMNWVRQAPGKGLEWVSAISMSGESTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 3 - protein, 247 aa, synthetic construct
QVQLQESGGGLVQPGRSLRLSCAASGFTFSSYAMHWVRQAPGKGLEWVSAISSSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGHKFPVSWYQQYPGKAPKLLIYKNLLRPSGVPDRFSGSKSGTSASLAITGLQAEDGADYYCQSYDSSLRVVFGGGTKVTVLG

SEQ ID NO: 4 - protein, 243 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 5 - protein, 247 aa, synthetic construct
QVQLQESGGGLVQPGRSLRLSCAASGFTFSTYAMTWVRQAPGKGLEWVSSISSSGESTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 6 - protein, 247 aa, synthetic construct
QVQLQESGGGLVQPGRSLRLSCAASGFTFSTYAMAWVRQAPGKGLEWVSEISSSGSRTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 7 - protein, 247 aa, synthetic construct
QVQLQESGGGLVQPGRSLRLSCAASGFTFSSYAMHWVRQAPGKGLEWVSAISSSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGLMHNVSWYQQYPGKAPKLLIYKSSSRPSGVPDRFSGSKSGTSASLAITGLQAEDGADYYCQSYDSSLRVVFGGGTKVTVLG

SEQ ID NO: 8 - protein, 243 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 9 - protein, 45 aa, synthetic construct
TTTPAPRPPTPAPTIASQPLSLRPEACRPAAGGAVHTRGLDFACD

SEQ ID NO: 10 - protein, 27 aa, synthetic construct
FWVLVVVGGVLACYSLLVTVAFIIFWV

SEQ ID NO: 11 - protein, 41 aa, synthetic construct
RSKRSRLLHSDYMNMTPRRPGPTRKHYQPYAPPRDFAAYRS

SEQ ID NO: 12 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 42 aa, synthetic construct
KRGRKKLLYIFKQPFMRPVQTTQEEDGCSCRFPEEEEGGCEL

SEQ ID NO: 14 - protein, 21 aa, synthetic construct
MALPVTALLLPLALLLHAARP

SEQ ID NO: 15 - protein, 24 aa, synthetic construct
IYIWAPLAGTCGVLLLSLVITLYC

SEQ ID NO: 16 - protein, 473 aa, synthetic construct
QVQLQESGGGLVQPGRSLRLSCAASGFTFSSYAMHWVRQAPGKGLEWVSAISGSGGSTYYADSVKGRFTISRDNSKNTLYLQMNSLRAEDTAVYYCAKDRRGSHADAFDVWGQGTLVTVSSGGGGSGGGGSGGGGSQSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 5 aa, synthetic construct
DYEMH

SEQ ID NO: 18 - protein, 17 aa, synthetic construct
ALDPKTGDTAYSQKFKG

SEQ ID NO: 19 - protein, 6 aa, synthetic construct
FYSYTY

SEQ ID NO: 20 - protein, 16 aa, synthetic construct
RSSQSLVHSNRNTYLH

SEQ ID NO: 21 - protein, 7 aa, synthetic construct
KVSNRFS

SEQ ID NO: 22 - protein, 9 aa, synthetic construct
SQNTHVPPT

SEQ ID NO: 23 - protein, 115 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 24 - protein, 113 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKR

SEQ ID NO: 25 - protein, 16 aa, synthetic construct
RSSQSLVHSNGNTYLH

SEQ ID NO: 26 - protein, 115 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 27 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 28 - protein, 469 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - protein, 467 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 511 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 5 aa, synthetic construct
DYEMH

SEQ ID NO: 32 - protein, 16 aa, synthetic construct
AIHPGSGDTAYNQRFK

SEQ ID NO: 33 - protein, 6 aa, synthetic construct
FYSYAY

SEQ ID NO: 34 - protein, 16 aa, synthetic construct
RSSQSLVHSNGNTYLQ

SEQ ID NO: 35 - protein, 7 aa, synthetic construct
KVSNRFS

SEQ ID NO: 36 - protein, 9 aa, synthetic construct
SQSIYVPYT

SEQ ID NO: 37 - protein, 115 aa, synthetic construct
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSA

SEQ ID NO: 38 - protein, 113 aa, synthetic construct
DIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 39 - protein, 6 aa, synthetic construct
SGYNWH

SEQ ID NO: 40 - protein, 16 aa, synthetic construct
YIHYTGSTNYNPALRS

SEQ ID NO: 41 - protein, 9 aa, synthetic construct
IYNGNSFPY

SEQ ID NO: 42 - protein, 17 aa, synthetic construct
KSSQSLFNSGNQKNYLT

SEQ ID NO: 43 - protein, 7 aa, synthetic construct
WASTRES

SEQ ID NO: 44 - protein, 9 aa, synthetic construct
QNAYSFPYT

SEQ ID NO: 45 - protein, 118 aa, synthetic construct
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 46 - protein, 114 aa, synthetic construct
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 47 - protein, 247 aa, synthetic construct
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 48 - protein, 247 aa, synthetic construct
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 49 - protein, 247 aa, synthetic construct
DVQLQESGPDLVKPSQSLSLTCTVTGYSITSGYNWHWIRQFPGNKMEWMGYIHYTGSTNYNPSLRSRISITRDTSKNQFFLQLNSVTTDDTATYYCTRIYNGNSFPYWGQGTSVTVSSGGGGSGGGGSGGGGSDIVMTQSPSSLTVTPGEKVTMTCKSSQSLFNSGNQKNYLTWYQQRPGQPPKMLIYWASTRESGVPDRFTGSGSGTDFTLTISSVQAEDLAVFYCQNAYSFPYTFGGGTKLEIKR

SEQ ID NO: 50 - protein, 247 aa, synthetic construct
QVQLVQSGAEVKKPGASVKVSCKASGYTFTSYTMHWVRQAPGQGLEWMGYINPASGYTNYNQKFKDRVTMTRDTSTSTAYMELSSLRSEDTAVYYCARIYYGNSFAYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPLTFGGGTKVEIKR

SEQ ID NO: 51 - protein, 473 aa, synthetic construct
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 52 - protein, 471 aa, synthetic construct
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 53 - protein, 473 aa, synthetic construct
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 471 aa, synthetic construct
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - protein, 15 aa, synthetic construct
EPKSCDKTHTCPPCP

SEQ ID NO: 56 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 12 aa, synthetic construct
ESKYGPPCPPCP

SEQ ID NO: 58 - protein, 28 aa, synthetic construct
MFWVLVVVGGVLACYSLLVTVAFIIFWV

SEQ ID NO: 59 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 466 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 438 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 242 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 63 - protein, 118 aa, synthetic construct
QVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSS

SEQ ID NO: 64 - protein, 219 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAII